(19) United States
(12) Patent Application Publication
(10) Pub. No.: US 2024/0174743 A1
(43) Pub. Date: May 30, 2024

(54) CHIMERIC OPSIN GPCR PROTEINS

(71) Applicant: UNIVERSITÄT BERN, Bern (CH)

(72) Inventors: Sonja KLEINLOGEL, Hinterkappelen (CH); Michiel VAN WYK, Hinterkappelen (CH)

(21) Appl. No.: 17/756,547

(22) PCT Filed: Nov. 30, 2020
(86) PCT No.: PCT/EP2020/083960

§ 371 Date: May 26, 2022

(30) Foreign Application Priority Data
Nov. 29, 2019 (CH) 01509/19

(51) Int. Cl. C07K 16/28; A61K 48/00; A61P 27/02; C12N 15/86

(52) U.S. Cl. CPC C07K 16/28; A61K 48/0033; A61K 48/0066; A61P 27/02; C12N 15/86; C07K 2319/60; C12N 2750/14143; C12N 2830/007

(57) ABSTRACT

A chimeric opsin GPCR protein comprising a light-sensitive upstream opsin portion and a target GPCR portion comprising a chimeric CT is provided that expresses strongly and is targeted into the correct subcellular compartment of target cells. The chimeric opsin GPCR protein activates efficiently the native G-protein specific to the target GPCR pathway eliciting a physiological response comparable to the native target GPCR. Nucleic acid molecules encoding the chimeric opsin GPCR protein as well as a capsids, vectors, cells and carriers comprising or expressing the chimeric opsin GPCR protein are also provided. Furthermore, a method of genetically engineering a chimeric opsin GPCR protein and medical applications of the chimeric opsin GPCR protein are provided.

### SEQUENCE LISTING

The instant application contains a Sequence listing which has been submitted electronically in ASCII txt format and is hereby incorporated by reference in its entirety. Said ASCII txt copy, created on Aug. 26, 2022, is named P081891US_Corrected_SL.txt and is 175,908 bytes in size.

### RELATED APPLICATIONS

This application is a U.S. national stage entry under 35 U.S.C. § 371 of international application no. PCT/EP2020/083960, filed Nov. 30, 2020, which designates the U.S. and claims the benefit of priority of Swiss application no. 01509/19 filed Nov. 29, 2019, incorporated herein by reference in its entirety.

### TECHNICAL FIELD

The present invention lies in the field of optogenetics and introduces further light sensitive chimeric opsin GPCR proteins. In particular, it relates to chimeric GPCR proteins comprising a light sensitive opsin and a further GPCR protein and the nucleic acid molecules encoding them as well as methods of engineering such nucleic acid molecules. The invention further relates to a capsids, vectors and particles comprising the chimeric opsin GPCRs or the nucleic acid molecules encoding it as well as to therapeutic applications, e.g. to medicaments and methods of gene therapy, based on light activation and coupling into a deliberately selected signaling cascade of the chimeric opsin GPCR proteins. The invention relates in particular to opsin-mGluR6 chimeric proteins and their use for gene therapeutic treatment of human or animal patients suffering from loss of vision due to photoreceptor degeneration.

### BACKGROUND ART

About one in 3000 people suffer from a genetic mutation that results in photoreceptor degeneration and blindness. Despite photoreceptor loss, the downstream retinal neurons remain largely intact. Recent studies showed that light sensitivity and functional vision can be restored if light-activatable proteins are introduced directly to the surviving retinal tissue after photoreceptor loss (Lagali P. S. et al., 2008; van Wyk M et al., 2015; Cehajic-Kapetanovic J. et al. 2015).

WO 2012/174674 discloses chimeric light sensitive G-protein coupled receptor (GPCR) proteins comprising intracellular domains of mGluR6. One such chimeric GPCR protein comprises the light sensitive GPCR melanopsin and IL2, IL3 and the CT of mGluR6 and is also referred to as Opto-mGluR6 (van Wyk M et al., 2015). Advantageously, Opto-mGluR6 proteins are light-activatable versions of the endogenous mGluR6 receptor and are therefore capable to couple light activation into the mGluR6 specific intracellular signaling cascade by activating a Galpha(o) G-protein, the native mGluR6 protein that is resident exclusively in the targeted ON-bipolar cells.

GPCRs are primary targets of the pharmaceutical industry (Sriram P. et al, 2018). The prior art comprises other approaches to enable optical activation of target-GPCR specific G-protein signaling cascades, as reviewed e.g. in Optogenetic user's guide to Opto-GPCRs (Kleinlogel S., 2016) or as e.g. applied with a chimeric rhodopsin MOPR opioid receptor protein termed opto MOR for a spatiotemporal control of opioid signaling and behavior (Siuda et al. 2015).

Thus, functionally active chimeric opsin GPCR proteins provide for dual functions: First, they are light activatable, i.e. they are light sensitive due to their light-sensitive opsin portion. Second, they couple light activation into the GPCR signaling pathway of the target GPCR protein.

GPCR proteins (G-protein-coupled receptor proteins; in short GPCRs) represent the largest superfamily of receptors in the human genome and are divided into five families (or classes) according to the GRAFS system of phylogenetic classification based on sequence homology and functional similarity (Schiöth, H. B. and Fredriksson R, 2005).

Class A is the largest and best understood family of the GPCR proteins. It is also named rhodopsin family after its “prototype”.

GPCRs of all families share a highly conserved tertiary structure and a similar mode of activation: All GPCRs comprise seven transmembrane domains (TM1 to TM7) connected by three extra- and three intracellular loops (ELs and ILs) of various lengths and an extracellular N-terminal domain (NT) and an intracellular C-terminal domain (CT).

Furthermore, most class A GPCRs and many or even the majority of GPCR proteins also of the other GPCR classes and in particular also of class C GPCRs comprise in addition to the seven transmembrane helices (TM1 to TM7) a helix eight (H8) downstream of TM7 (Bruno et al, 2012). H8 is a subdomain of the CT in the proximal region of the CT of GPCRs and not a transmembrane helix. Rather, H8 lies parallel and adjacent to the cytoplasmic surface of the cell membrane and is therefore sometimes referred to as an amphoteric helix.

GPCR receptor proteins physiologically interact with heterotrimeric G-proteins consisting of three functional subunits, namely a G-alpha, a G-beta and a G-gamma subunit. G proteins have been classified into four subfamilies, namely, Gs, Gi/o, Gq/11, and G12/13, based on the structural similarity of their alpha subunits and on the type of modulatory response they induce. Each GPCR preferentially couples to one subfamily of G proteins, thus stimulating preferentially one signaling cascade. Structural interaction between GPCR receptors and G-proteins have been subject of many studies as e.g. summarized by Moreira I, 2014. The four major classes of G-proteins are themselves divided into subclasses. For example mGluR6 in its activated stage binds to Galpha(o) which is a subclass of Galpha(i/o).

Exemplary endogenous G proteins present in the physiological cellular environment of exemplary parent opsins are G(alpha)q for melanopsin, G(alpha)t for cone opsins and G(alpha)s for jellyfish opsin.

Remarkably, the members of any GPCR family despite their similar three dimensional structure exhibit virtually no sequence similarity to those of other families (Kleinlogel, 2016) with the exception of a few conserved amino acids and short motifs identified by structural alignment of the three major GPCR classes A, B and C that are important for function, signal transduction and 3D conformational stability (Schwartz et al. 2006, Nygaard et al. 2013).

In the past decade, domains have been exchanged between two GPCRs, in particular between light-activated opsin-GPCRs and ligand-activated non-opsin GPCR proteins providing a few examples of functionally active chimeric GPCR proteins. These chimeric GPCRs are activated by a ligand or in particular by light, which is characteristic of a first GPCR protein, and which couple this signal activation to a second GPCR protein by binding to a Galpha protein that is characteristic of the second GPCR protein (Kleinlogel, 2016; Morri et al., 2018; Siuda et al., 2015). Such exchange of domains, despite the lack of sequence similarity, is rendered possible by sequence alignment of GPCR proteins and domain identification using the conserved motifs described below as landmarks.

Some of the above-mentioned highly conserved amino acids and short motifs are located at a junction between an intracellular and a transmembrane domain, in particular:
a highly conserved E(D)RY (SEQ ID NO 80) motif at the junction (c) between TM3 and IL2,
a glutamate residue (E) at the junction (f) between IL3 and TM6,
   which together form an “ionic lock” between TM3 and TM6 that stabilizes the inactive state of GPCRs;
a NPxxY motif (SEQ ID NO 81) at the junction (g) between TM7 and the proximal end of the CT, in particular between TM7 and a proximal end of a helix 8 (H8).

Both, the ionic lock between TM3 and TM6 formed by the E(D)RY (SEQ ID NO: 80) site at the junction of TM3 and IL2 and the glutamate residue at the junction of IL3 and TM6 and the NPxxY motif (SEQ ID NO: 81) at the end of TM7 provide important structural constraints which rearrange in response to a signal, e.g. upon photoisomerization of retinal by light activation or upon ligand binding, thereby forming an activated conformation of GPCR proteins (Fritze et al. 2003).

An NR(K)Q (e.g. HPK or HEP) sequence (SEQ ID NO 82) is a further highly conserved motif at the proximal end of H8 in the proximal region of the CT of most GPCRs, in particular of class A and class C GPCRs. The NR(K)Q motif (SEQ ID NO: 82) and the proximal region of the CT that often comprises an H8 appear to be relevant for a conformational switch of GPCR proteins upon activation, i.e. triggered by ligand binding or in case of opsins by light absorption. Furthermore, the NR(K)Q motif (SEQ ID NO: 82) and the proximal region of the CT are considered important for arrestin binding to control the GPCRs activity (e.g. Sato T, 2019).

The chimeric opsin GPCR proteins exhibit the highly conserved tertiary structure of GPCR receptor proteins comprising seven transmembrane domains and comprising the highly conserved motifs such as in particular the E(D)RY motif (SEQ ID NO: 80) at a distal end of TM3 and the NPxxY motif (SEQ ID NO: 81) at a distal end of TM7.

Further partially conserved structural elements and motifs include:
One or two palmitoylation sites which directly follow H8 in distal direction and correspond to C322 and C323 of bovine rhodopsin (Ovchinnikov Yu A, 1988). The covalent modification of amino acid residues connected to a palmitic acid results in an anchor of the distal end of H8 to the membrane. In most opsins, a cysteine residue at the distal end of H8 is palmitoylated. The palmitoylation of amino acid residues at the distal end of H8 is considered to be mainly involved in GPCR membrane localization, lipid raft recruitment and protein stabilization. Some exceptional opsins, e.g. the cone opsins OPN1MW and OPN1LW do not comprise a palmitoylated amino acid at the distal end of H8.
Phosphorylation sites in the C-terminus which are generally located beyond the palmitoylation site in distal direction. Phosphorylation sites typically refer to phosphorylated serine or threonine or sometimes tyrosine residues. Phosphorylation sites are involved in the desensitization and internalization for turn-over of a GPCR receptor. They also determine the binding preferences of activity regulators such as in particular G protein-coupled receptor kinase (GRK) and arrestin, which influence the kinetics of the G-protein signaling. For example, melanopsin has a particularly long C-terminus with multiple phosphorylation sites and in particular some distal phosphorylation sites have been shown to render cessation of signaling slow (cf. Mure L. et al, 2016) in favor of the temporally integrative physiological function of this opsin entraining the circadian clock.

Opsins comprise further conserved motifs. In particular, there are two conserved motifs present in a chromophore pocket for covalently binding a chromophore. The chromophore of all animal opsins is 11-cis-retinal. There are two highly conserved motifs in the chromophore pocket:
a conserved lysine (K) in TM7 that is covalently linked to the chromophore 11-cis-retinal via a Schiff base,
a negative counter-ion (E) in TM3 that stabilizes the Schiff base binding of 11-cis-retinal.

In other words, the chromophore pocket comprises a Schiff base, a lysine residue in TM7, which covalently binds a chromophore and it further comprises a negative counter-ion in TM3.

There is still a need for further light sensitive chimeric GPCR proteins that are genetically engineered to exhibit particularly favorable properties for their use in medical therapy, such as for restoration and modulation of physiological function or regulation of GPCR receptor activity. Particularly favorable properties include e.g. conformational stability, physiologically suitable kinetic properties, significant magnitude of the response and efficient intracellular trafficking to the physiological localization of the target GPCR.

There is also still a need for light sensitive opsin GPCR chimera comprising further target GPCRs for further medical applications. Non-limiting examples of desired therapeutic targets for light-activatable GPCR receptor proteins in further medical applications include e.g. treatment of pain, heart failure, anxiety or color vision.

Furthermore, there is still a need for further guidance in the design of functionally active chimeric light sensitive opsin GPCR proteins obtainable by genetic engineering. Simple and efficient genetic engineering methods yielding light sensitive opsin GPCR proteins are still desired not only for physiological restoration of degenerated photoreceptors by gene therapy but also for manipulation of the activity of other target GPCR proteins which may be suitable for further applications.

### SUMMARY OF THE INVENTION

Hence, it is a general object of the invention to meet such needs as mentioned above and provide chimeric GPCR proteins between an opsin and a further GPCR protein, termed target GPCR protein.

It is a particular object of the invention to provide further chimeric opsin mGluR6 proteins exhibiting one or more particularly favorable property for their use in gene therapy for patients with partial or complete loss of vision, e.g. due to a lack or an insufficiency of light sensitive signaling activity provided by their natural photoreceptors.

It is a particular object of the invention to engineer chimeric opsin GPCR proteins with one or more particular favorable property including e.g. efficient expression in their target cells, such as e.g. efficient expression of opsin-mGluR6 chimera in ON bipolar cells; their efficient and specific intracellular sorting in their target cell, such as e.g. dendritic trafficking of opsin-mGluR6 chimera into the mGluR6 signalosome in ON bipolar cells; efficient coupling into the native GPCR G-protein signaling pathway of the target cell, such as e.g. coupling to Gao in bipolar cells; exhibiting conformational stability, capability of generating physiological output with physiological kinetics and low desensitization.

It is a further object of the invention to provide a simple principle of chimeric opsin-GPCR design applicable to any target GPCR to enable light activation of the target-GPCR specific G-protein signaling cascade. This object addresses the need of a simple method for designing and genetically engineering chimeric opsin GPCR proteins in particular for their use in physiological manipulation of cells or in gene therapy or in other medical and pharmacological applications.

It is a further object of the invention to modulate a selected cellular response by a chimeric opsin GPCR in particular also for use in optogenetic therapeutics allowing to re-establish or improve a therapeutic GPCRs signaling pathway with unparalleled specificity, that is not achieved with current pharmaceuticals.

Non-limiting examples of this object of the invention include providing further chimeric opsin GPCRs such as e.g. chimeric opsin opioid receptor GPCRs, chimeric opsin hydroxytryptamine receptor (HT) GPCRs, chimeric GABA(B) receptors, and chimeric opsin beta adrenergic receptor GPCR proteins for use as a medicament.

The objects of the invention accordingly further include providing genetically engineered nucleic acid molecules encoding the designed chimeric opsin GPCR proteins, methods of genetically engineering and expressing nucleic acid molecules comprising fusion genes encoding the chimeric opsin GPCR proteins as well as medical, in particular, gene therapeutic products and methods based on the chimeric opsin GPCR proteins or nucleic acids molecules encoding them, respectively.

In order to implement these and still further objects of the invention, which will become more readily apparent as the description proceeds, the chimeric opsin GPCR protein and aspects of the invention related to it are manifested by the features described below.

In a first aspect of the invention a chimeric opsin GPCR protein is provided:

The chimeric opsin GPCR protein comprises seven transmembrane domains (TM1 to TM7) connected by extra- and intracellular loops (ELs and ILs) of various lengths.

The chimeric opsin GPCR protein comprises a light sensitive opsin portion of an upstream opsin and a second GPCR portion termed target GPCR portion of a second GPCR protein termed target GPCR protein.
The target GPCR portion comprises a C-terminal domain of the target GPCR (target GPCR CT, or shorter target CT). The upstream opsin portion comprises a chromophore pocket covalently binding a chromophore.
The upstream opsin portion further comprises a truncated C-terminal domain. This truncated CT of the upstream opsin has a truncation site positioned at or downstream of a distal end of a proximal region of the upstream opsin CT (O-CT-proximal region).
This O-CT-proximal region comprises a NR(K)Q motif (SEQ ID NO: 82) and the following 7 to 13 amino acids in distal direction
whereby said chimeric opsin GPCR protein comprises a chimeric C-terminal domain (chimeric CT).
The target-GPCR-CT is positioned downstream of the truncated opsin CT.

As further detailed below, the proximal region of the CT of most opsins comprises a subdomain termed helix 8 (H8) beginning with the NR(K)Q motif (SEQ ID NO: 82) and ending at an amino acid position approx. 7 to 13 amino acids downstream of it. Recently, the potentially important role of H8 in G-protein binding and activity modulation has been indicated by structural data (Ahn et al., 2010, Sato, 2019, Tsai, et al., 2018). On the other hand G-protein binding specificity is modulated by more distal regions of the CT, as the inventors found. H8 is often anchored at its distal end by one or multiple palmitoylation sites into the cytoplasmic membrane. The distal end of the O-CT-proximal region may be positioned at any amino acid position of approx. 10 amino acids, i.e. between 7 to 13 or 8 to 12 or 9 to 11 amino acids, downstream of the distal end of the NR(K)Q motif (SEQ ID NO: 82). In the literature, H8 is sometimes regarded as an additional GPCR domain positioned between the TM7 and the CT domains and sometimes it is regarded as a subdomain of the CT. In this text, H8 is referred to as a subdomain of the CT.

Accordingly, in some embodiments, the distal end of the O-CT proximal region is positioned at a position selected from the group comprising
at a distal end of a helix 8 (H8)
at a palmitoylation site or
at a position corresponding to a palmitoylation site in bovine rhodopsin.

The second GPCR protein is termed target GPCR because the light activated chimeric opsin GPCR no longer couples its activation into the natural opsin signaling pathway but instead into the signaling pathway of a deliberately selected target GPCR that is used for the design and construction of the chimeric opsin GPCR by genetic engineering.

It is the inventor's unexpected finding that a C-terminal domain comprising the proximal region of the upstream opsin CT (O-CT-proximal region) together with the CT of a target GPCR (target-CT) suffices to efficiently couple light activation of the chimeric opsin GPCR protein to the signaling cascade of the target GPCR. This relates in particular, to subcellular trafficking, response kinetics, G-protein binding specificity and interaction with intracellular binding partners of the chimeric opsin GPCR which mimics the corresponding characteristics of the target GPCR protein.

This was first observed with chimeric opsin GPCRs comprising an upstream opsin that was truncated at a palmitolytion site at the distal end of the O-CT-proximal region and spliced together with the target CT.

It is indeed a striking surprise that chimeric opsin GPCR proteins comprising the truncated upstream CT and the target CT, i.e. comprising a chimeric CT, achieve coupling into the signaling pathway of the target GPCR even in the absence of any intracellular loop of the target GPCR protein with a response that corresponds to or excels the physiological response of the native target GPCR protein. The truncated opsin CT with the O-CT-proximal region embedded in it causes a significant increase in the efficiency of G-protein activation by the target CT.

The opsin GPCR proteins of the current invention are designed to comprise the O-CT-proximal region to enhance light-activated G-protein activation. On the other hand, they are designed to not include the entire CT of the upstream opsin. Instead, regions of the upstream opsin CT that are located distal to the O-CT-proximal region and that are involved in subcellular trafficking, kinetic regulation and—as shown in this invention—G-protein specificity, are excluded from the chimeric opsin GPCR. In other words, only the target GPCR portion of the chimeric opsin GPCR comprises C-terminal regions that induce these physiological activities. Thereby, interference by molecular information derived from the upstream opsin CT with those of the target GPCR CT are avoided and thus, G-protein specificity, specificity of the subcellular localization, and kinetic regulation pertaining to the target GPCR are enhanced. Thus, the chimeric opsin GPCR proteins, upon light-activation, mimic the response of the target GPCR and light activation is efficiently coupled to the signaling cascade of the target GPCR.

Intracellular loops of the target GPCR protein may optionally be added to the chimeric opsin GPCR of the invention but they are not required. Accordingly, the present invention enables the genetic engineering of light-activatable chimeric opsin GPCR proteins—if so desired—with only one single gene fusion site at the truncation site of the upstream opsin CT spliced together with the proximal end of the target CT.

Thus, the invention provides further opsin GPCR chimeras requiring only minimal genetic engineering, that express strongly and in the correct subcellular compartment of target cells and efficiently activate the native G-protein pathway of target cells eliciting a response that mimics the physiological response of the target GPCR.

Surprisingly, the chimeric opsin GPCR proteins described here, even in the absence of all intracellular loops emulate the physiological response specific to the target GPCR compared to chimeric light sensitive GPCRs available in the prior art such as e.g. opto-mGluR6 (van Wyk et al. 2015).

In some embodiments, the target GPCR protein is a metabotropic glutamate receptor 6 (mGluR6). mGluR6 is the endogenous GPCR protein in retinal ON bipolar cells which in the healthy physiological visual signaling cascade is activated by glutamate and couples its activation to the visual signal cascade by binding to a Galpha(o) protein. ON bipolar cells are retinal neurons directly downstream of the physiological photoreceptor cells in the visual signal cascade.

The first aspect of the invention further relates to a chimeric C-terminal peptide comprising an O-CT-proximal region and a target GPCR CT or a functional variant thereof. In some embodiments of the chimeric C-terminal peptide, such functional variant of the target GPCR CT is a distal C-terminal fragment of the target GPCR with a proximal end at the distal end of H8 or at the palmitoylation site. In some embodiments of the chimeric C-terminal peptide, the C-terminal fragment additionally comprises H8 of the target GPCR CT.

A second aspect of the invention relates to a nucleic acid molecule encoding the chimeric opsin GPCR protein and the chimeric C-terminal peptide according to the first aspect of the invention.

A third aspect of the invention relates to a an AAV capsid for medical use of transferring the nucleic acid molecule according to the second aspect encoding the chimeric opsin GPCR according to the first aspect into a target cell. The third aspect further relates to a nucleic acid molecule encoding the capsid.

An independent invention relates to a novel, rationally designed adeno-associated viral (AAV) capsid for packaging and transport of a transgene to target cells and to a nucleic acid molecule encoding the capsid. The independent invention relates in particular to the transfer of the nucleic acid molecule encoding the chimeric opsin GPCR into a target cell.

A fourth aspect of the invention relates to a vector comprising a nucleic acid molecule according to the second aspect of the invention encoding the chimeric opsin GPCR protein or the chimeric C-terminal peptide according to the first aspect of the invention.

A fifth aspect of the invention relates to particles, in particular nano particles, vesicles, cells—in particular excluding germ cells—and animals comprising or expressing nucleic acid molecules according to the second aspect, or vectors according to the third aspect or comprising the chimeric opsin GPCR according to the first aspect of the invention.

A sixth aspect of the invention relates to a method of genetically engineering the nucleic acid molecules of the second aspect encoding the chimeric opsin GPCR proteins of the first aspect of the invention.

A seventh aspect of the invention relates to the products related to the chimeric opsin GPCR proteins according to the invention for medical use. In particular the seventh aspect relates to chimeric opsin GPCR proteins according to the first aspect, or a nucleic acid molecule encoding said opsin GPCR protein according to the second aspect or a capsid or a nucleic acid molecule encoding said capsid both according to the third aspect, or a vector according to the fourth aspect, or a particle, vesicle or cell for use in medical therapy. The seventh aspects further relates to medicaments and methods of treatment of using the above mentioned products based on the chimeric GPCR proteins according to the invention.

Some embodiments of the above-mentioned aspects of the invention relate in particular to chimeric opsin mGluR6 proteins or chimeric opsin GPCRs comprising two opsins and their use for gene therapy of patients suffering from partial or complete loss of vision, in particular due to photoreceptor degeneration.

### BRIEF DESCRIPTION OF THE DRAWINGS

The invention will be better understood and objects other than those set forth above will become apparent when consideration is given to the following detailed description thereof. Such description makes reference to the annexed drawings:

FIG. 1: General structure of an opsin. Figure discloses SEQ ID NOS 80-82, respectively, in order of appearance.

FIG. 2: Scheme of an exemplary chimeric opsin GPCR. Figure discloses SEQ ID NOS 80-82, respectively, in order of appearance.

FIG. 3: Exemplary embodiment of a chimeric opsin mGluR6. Figure discloses SEQ ID NOS 80-82 and 92-99, respectively, in order of appearance.

FIG. 4: Exemplary embodiments of chimeric opsin GPCRs target to the cell membrane.

FIG. 5: Exemplary embodiments of chimeric opsin mGluR6 with a chimeric C-terminus increased light-activated currents mediated by opsin-mGluR6s as compared to the parent opsin.

FIG. 6: Example of in vitro functional screening of chimeric opsin GPCRs using HEK-GIRK cells.

FIG. 7: Plate reader experiments probing for G-protein re-targeting and pathway selectivity of exemplary embodiments of chimeric opsin GPCRs.

FIG. 8: Correct in vivo trafficking into the ON-bipolar cell dendrites and the mGluR6 signalosome of exemplary embodiments of chimeric opsin-mGluR6 variants.

FIG. 9: Exemplary embodiments of chimeric opsin-mGluR6 GPCRs render isolated ON-bipolar cells directly light sensitive.

FIG. 10: In vivo measurements of visual acuities of blind mice that were treated by an AAV gene therapy with exemplary embodiments of chimeric opsin mGluR6 variants.

FIG. 11: Ex vivo Light responses recorded from retinal ganglion cells in blind rd1 retinas treated with exemplary embodiments of a chimeric opsin-mGluR6.

FIG. 12: Micrograph of vertical cryosections through the retinas from two blind rd1 retinas after an intravitreal gene therapy with an exemplary embodiment of an AAV expressing an exemplary embodiment of a chimeric opsin-mGluR6.

FIG. 13: Light-induced currents measured with the whole-cell patch-clamp method of an exemplary JSR1 (S186F)palm-beta2AR chimera expressed in HEK293-GIRK cells.

### DEFINITIONS AND DETAILED DESCRIPTION

Unless defined otherwise, all technical and scientific terms used herein have the same meaning as commonly understood by a person skilled in the art to which this invention pertains. Otherwise, certain terms used herein have the meanings as set forth in the description below.

As used herein and in the claims, the singular forms “a”, “an”, and “the” include plural forms unless the context clearly dictates otherwise.

Unless otherwise indicated, the term “at least” preceding a series of elements is to be understood to refer to every element in the series.

In this text, the term “comprise” or “comprising”, is defined to include a stated element or step or group of elements or steps. In this text, the term “comprise” or “comprising” does not generally exclude any other element or step or group of elements or steps. Furthermore, term “comprise” or “comprising” in this text, also pertains to the exact statement of element or step or group of elements or steps. Only in this latter case of the meaning of the term “comprise” or “comprising”, it is in fact congruent with the term “consisting of” that generally excludes any element, step, or ingredient that is not specified in the claim.

A first aspect of the invention relates to chimeric opsin GPCR proteins. The phrase “chimeric opsin GPCR protein” comprising a light-sensitive upstream opsin portion and a second GPCR portion (target-GPCR portion) of a second GPCR protein refers to a light-sensitive genetically engineered GPCR protein comprising a tertiary structure that is a characteristic, conserved structure among GPCR proteins as described above. The chimeric opsin GPCR proteins—alternatively termed (chimeric) opsin GPCR receptors or (chimeric) opsin GPCRs or chimeric GPCRs—and the chimeric nucleic acid molecules or chimeric (fusion) genes encoding them are obtainable in particular by genetic engineering techniques known in the art including e.g. cutting parent genes and ligation of selected parent gene portions or e.g. synthesis of nucleic acid molecules encoding the chimeric opsin GPCR protein or fragments thereof. The upstream opsin portion confers light sensitivity and activation upon exposure to light to the chimeric GPCR receptor. The second GPCR portion is termed target GPCR portion, because it provides for coupling the light activation of the chimeric opsin GPCR protein to the G-protein specific to the physiological signal pathway of the target GPCR as explained above. Exemplary target GPCRs include e.g. β-adrenergic receptor, GABA(B) receptor, MOR, mu opioid receptor, serotonine receptors such as 5-HT7, a second opsin such as OPN1 and metabotrobic glutamate receptors (mGluRs), such as mGluR6 or mGluR5.

In this text, the opsin and target GPCR from which the chimeric opsin GPCR is derived are referred to as parent GPCRs. Thus, e.g. a chimeric melanopsin mGluR6 protein accordingly is designed and engineered by using portions of the parent GPCR melanopsin and of the parent mGluR6. As evident from the context, the term parent GPCR refers to either the parent GPRC protein or the parent GPCR gene or both.

In this text, the term GPCR protein refers to a G-protein-coupled receptor protein.

In this text, the term “opsin” refers to the light sensitive members of the Class A GPCR proteins, and in particular it refers to physiological, natural opsins. The term “opsin” in some embodiments may also include functionally active, i.e. light sensitive opsins that are variants of natural opsins that generally comprise the conserved GPCR 3D structure and conserved motifs as described further below. Such genetic opsin variants are encoded by a nucleic acid molecule derived e.g. from a mutated opsin gene or derived from a genetically engineered, e.g. a chimeric opsin gene encoding a functional, light-sensitive opsin.

Similarly, the term target GPCR generally refers to a physiological, naturally occurring target GPCR and in some embodiments refers to variants of natural target GPCRs, such as variants of mGluR6, that generally exhibit the above-mentioned conserved GPCR 3D structure and conserved motifs and that are functional, i.e. capable of efficiently coupling activation into the signaling pathway of the target GPCR. Such variants of target GPCRs are encoded by a nucleic acid molecule derived e.g. from a mutated target GPCR gene or derived from a genetically engineered, e.g. chimeric target GPCR gene encoding a functionally active target GPCR protein.

Accordingly, in this text, the term domain or subdomain in some embodiments may include besides physiological natural domains of a GPCR protein a genetic variant of a domain or subdomain, such as e.g. a mutated or a genetically engineered, e.g. a chimeric or a synthetic domain or subdomain which functionally mimics a natural GPCR domain or subdomain.

Each domain of the upstream opsin portion is encoded by a gene fragment derived from a gene encoding an upstream opsin (upstream opsin gene) or a genetic variant thereof. Each domain of the target GPCR portion is encoded by a gene fragment derived from a target GPCR gene or a genetic variant thereof. Together all the domains of the chimeric opsin GPCR that are encoded by upstream opsin gene fragments are referred to as the upstream opsin portion and analogously together all the domains or subdomains encoded by target GPCR gene fragments are referred to as target GPCR portion. In some embodiments, the upstream opsin portion and or the target GPCR portion may be derived from one or more, in particular from two or three parent genes.

Target GPCRs may be selected from any GPCR class, in particular from classes A, B or C, more particularly from class A or C. Exemplary target GPCRs of class A include e.g. cone opsins, serotonin receptors (e.g. 5-HT7), mu opioid or β-adrenergic receptor. Exemplary target GPCRs of class B include e.g. the glucagon receptor(GCGR) and other hormonal receptors. Exemplary target GPCRs of class C include e.g. metabotropic glutamate receptors (mGluRs, e.g. mGluR6, mGluR5) or GABA_{B} receptor).

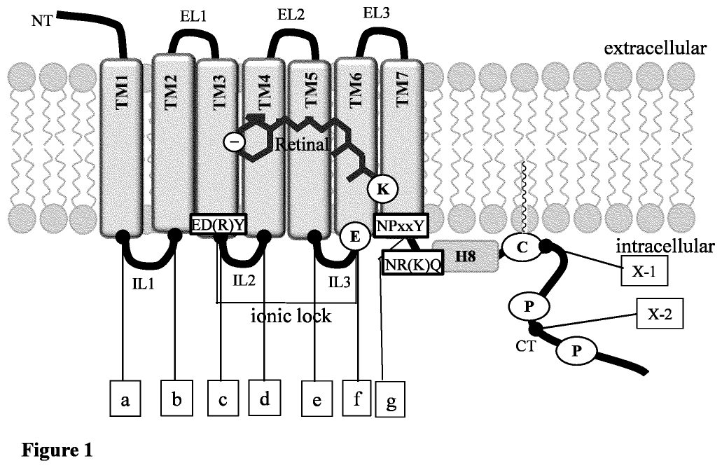

There are six junctions forming the transition between transmembrane and intracellular domains of the GPCR proteins, and correspondingly between the gene fragments encoding them, termed junction (a) between TM1 and IL1, junction (b) between IL1 and TM2, junction (c) between TM3 and IL2 and so forth up to junction (g) between TM7 and CT as shown in FIG. 1.

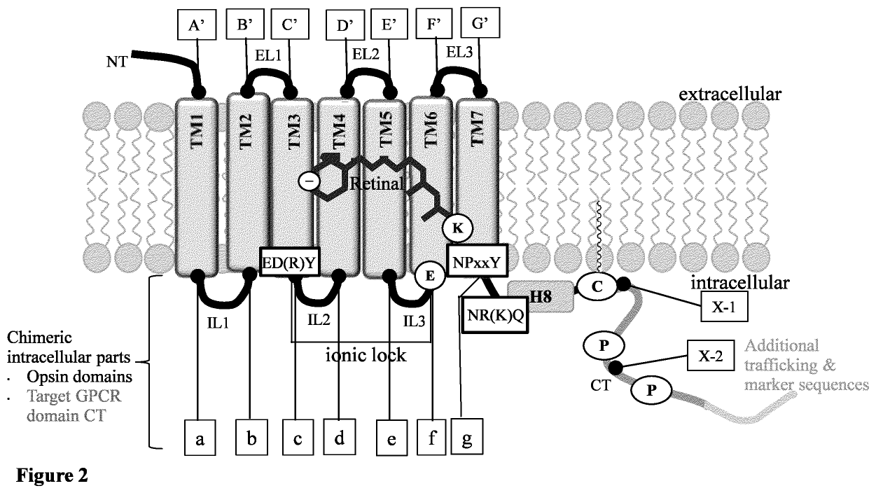

Analogously, there are also six junctions forming the transition between transmembrane and extracellular domains of the GPCR proteins and the gene fragments encoding them, termed junction (A′) between NT and TM1, junction (B′) between TM2 and EL1, junction (C′) between EL1 and TM3, and so forth up to junction (G′) between EL3 and TM7 as shown in FIG. 2.

In this text, the term “conserved motif” as used commonly in the art is not restricted to a motif consisting of the exactly same e.g. 3 to 5 amino acids that are commonly recited when referring to a specific conserved motif. Rather, each of these conserved motifs is named after a particularly frequent prototype sequence representing several alternatives such as e.g. included below in Table 1.

In addition, functional variants are derivable of conserved motifs. For example, functional variants of the E(D)RY motif (SEQ ID NO: 80) (ERY and DRY) include DRIY (SEQ ID NO 83), NRIY (SEQ ID NO 84) or NRY all of which yield light-sensitive opsin-mGluR6 chimeric GPCRs (cf. WO 2012/174674). Exemplary functional variants of conserved motifs are also shown in Table 2 below.

Evidently, several conserved motifs occur at or around junctions between TM domains and intracellular domains, i.e. at or around the junctions between the transmembrane helices (TM1 to TM7) and the intracellular loops (IL1 to IL3) and the C-terminus (CT). However, not all junctions necessarily comprise a highly conserved motif.

In this text, the term NPxxY motif (SEQ ID NO: 81) refers to a conserved motif at a distal end of TM7, i.e. around the TM7/CT junction of a GPCR protein, in particular of a parent upstream opsin or of a parent target GPCR or of a chimeric opsin GPCR, and is defined by fulfilling one or more of the following criteria:
A) it is a sequence of 5 consecutive amino acids of the sequence NPxxY (SEQ ID NO: 81), according to the one letter code for amino acids wherein x corresponds to any amino acid residue;
B) it is a sequence corresponding to the NPxxY motif (SEQ ID NO: 81) of a particular GPCR as is listed e.g. in Table I below;
C) it is a sequence of 5 amino acids that in an alignment of the amino acid sequence of a parent GPCR, in particular a parent upstream opsin, with the amino acid sequence of bovine rhodopsin corresponds to the sequence of N(302)PxxY(136) (SEQ ID NO: 81) of bovine rhodopsin.

The NPxxY motif (SEQ ID NO: 81) is present and identifiable in all class A GPCRs and also in most other GPCRs albeit exhibiting considerable sequence variation (cf. e.g. Table 2 of Sato, 2019).

In this text, the term NR(K)Q motif (SEQ ID NO: 82) refers to a conserved motif in a proximal region of the CT a few amino acids downstream of the NPxxY motif (SEQ ID NO: 81) of a GPCR protein, in particular of a parent upstream opsin or of a parent target GPCR or of a chimeric opsin GPCR, and of the opsin CT around the proximal end of H8 and is defined by fulfilling one or both of the following criteria:
A) it is a sequence corresponding to the NR(K)Q motif (SEQ ID NO: 82) for a particular GPCR, in particular a parent upstream opsin, that is listed in Table I or in Davies et al. 2010;
B) it is a sequence of 3 to 4 consecutive amino acids that in an alignment of the amino acid sequence of a parent GPCR, in particular a parent upstream opsin, with the amino acid sequence of bovine rhodopsin corresponds to the sequence of N(310)KQ(312) of bovine rhodopsin.

The NR(K)Q motif (SEQ ID NO: 82) is present and identifiable in all class A GPCRs and also in most other GPCRs. In particular, the NR(K)Q motif (SEQ ID NO: 82) corresponds to a sequence of 3 to 4 consecutive amino acids that is identifiable by sequence alignment with bovine rhodopsin, albeit exhibiting considerable sequence variation. The NR(K)Q motif (SEQ ID NO: 82) includes such sequences as HPK or HPE or HKQ or HPR or IRK or DYK and others (Davies W. et al., 2010).

In this text, the terms “palmitoylation site” and “amino acid position corresponding to a palmitoylation site” (the latter is also named palmitoylation site for short) is defined by fulfilling one or more of the following criteria A, B, C and D. In particular, the term palmitoylation site fulfills one criterion, e.g. A, or it fulfills the two criteria B and C or B and D or C and D or the three criteria B, C and D. The criteria A to D are:
A) it is a palmitoylated amino acid residue in the CT of a parent GPCR, in particular a parent opsin;
B) it is a palmitoylatable amino acid residue in the CT of a parent GPCR, in particular the parent opsin, positioned at least 7 amino acids, in particular at least 8 or 9 or 10 or 11 or 12 or 13 amino acids, downstream of the distal end of the NR(K)Q motif (SEQ ID NO: 82) of the opsin selected from the amino acids cysteine (C), serine (S), threonine (T), tyrosine (Y) or tryptophan (W);
C) it is an amino acid residue in the CT of a parent GPCR, in particular the parent opsin, positioned between 7 and 13 amino acid residues, in particular between 8 and 12, or between 9 and 11 or at 10 amino acid residues, downstream of the distal end of the NR(K)Q motif (SEQ ID NO: 82);
D) it is an amino acid residue that in an alignment of the amino acid sequences of a parent GPCR, in particular the parent opsin, with bovine rhodopsin corresponds to C322 or C323 of bovine rhodopsin.

Notably, this last criterion, D, refers to amino acid residues positioned at the end of H8 that in fact are not palmitoylated but that are corresponding to palmitoylated C322 and C323 of rhodopsin in an amino acid sequence alignment. For example, in both human cone opsins hOPN1MW and hOPN1LW, these amino acid positions correspond to amino acid residues G338 und K339.

A palmitoylated amino acid residue at the distal end of H8—if present—favorably enhances membrane association of H8. Accordingly, in chimeric opsin GPCRs, comprising an upstream opsin with one or more palmitoylation site at the distal end of H8, at least one palmitoylation site is most preferably retained in the truncated upstream opsin CT. Accordingly, in this text reference to a truncation site at a palmitoylation site which fulfills criterion A or B above is positioned distally adjacent to the palmitoylation site unless indicated otherwise.

In this text, in the context of protein and nucleic acid molecules, the terms downstream and distal refer to the C-terminal direction or region in proteins and the 3′ direction or region in nucleic acid molecules, whereas the term upstream and proximal refer to the N-terminal direction or region in proteins and 5′ direction or region in nucleic acid molecules. The terms downstream of and distal to and accordingly also the terms upstream of and proximal to are used interchangeably.

In this text, the terms upstream of (or proximal to) a domain, subdomain, region, motif or site refers to position that is upstream of (or proximal to) the proximal end of said domain, subdomain, region, motif or site including a position that is proximally adjacent.

In this text, the terms downstream of (or distal to) a domain, subdomain, region, motif or site refers to position that is downstream (or distal to) the distal end of said domain, subdomain, region, motif or site including a position that is distally adjacent.

In this text, the phrase “at” a particular domain, subdomain, region, motif or site refers to a position within said domain, subdomain, region, motif or site. In this text, the phrase “at or distal to” a particular motif or site refers to a position within or distal to said domain, subdomain, region, motif or site. Mutatis mutandis is applicable for the phrases “at or downstream of”, “at or proximal to” and “at or upstream of”.

In this text, the phrase between two particular motifs or sites such as e.g. the phrase between the NR(K)Q motif (SEQ ID NO: 82) and the palmitoylation site, unless explicitly stated or apparent from the context includes the amino acid positions within these motifs or sites as long as they are retained or reconstituted in their original or a functionally equivalent version. Indeed, in some preferred embodiments comprising a splicing site between two conserved motifs or sites or between a conserved motif and another amino acid position, a preferred splicing site may be located within a conserved motif and after completion of the splicing manipulation the conserved motif or site or a functional equivalent thereof is retained or reconstituted.

In this text, the phrase at a proximal end or at an upstream end of a domain, subdomain, region, motif or site includes a position at the proximal (or upstream) end that is still fully or partially within said domain, subdomain, region, motif or site at its proximal (or upstream) end or that is proximally adjacent to the proximal (or upstream end) of said domain, subdomain, region, motif or site.

In this text, the phrase at a distal end or at a downstream end of a domain, subdomain, region, motif or site analogously includes a position at the distal (or downstream) end that is still fully or partially within said domain, subdomain, region, motif or site at its distal (or downstream end) or that is distally adjacent to said domain, subdomain, region, motif or site.

In this text, in the context of signaling pathways upstream and downstream refer to earlier and later steps of the pathway or cells or components that are involved at earlier and later steps, respectively.

In this text, the term (gene) splicing site and alternative or similar terms such as (gene) fusion site, truncation site or cutting and ligation site refers to a site where gene fragments of different origin, in particular derived from different parent GPCRs are joined.

Unless stated to the contrary, a gene splicing site (fusion site, cutting and ligation site, truncation site) that is positioned at a conserved motif or site at the end of the gene manipulation retains or reconstitutes the same or a functionally equivalent of the motif or design as described above. Even if a splicing site is defined to be located upstream/proximal or downstream/distal of a particular motif, site or amino acid position, said gene splicing site may located at or within such particular motif, site or amino acid if as a result of the completed gene manipulation (e.g. cutting, ligating, truncating fusing, splicing) the conserved motif or amino acid is restored or replaced by a functional derivative thereof such as described above including e.g. in some embodiments, a conservative amino acid substitution.

In this text, abbreviations of G proteins and their corresponding Galpha subunit may be used that generally follow the pattern G(i/o) for Galpha(i/o) or Ga(o) for Galpha(o) or Ga(q) for Galpha(q) as common in the art.

In this text, the term variant refers to a polypeptide or the gene encoding it that differs from a reference polypeptide, but retains essential properties. A typical variant of a polypeptide differs in its primary amino acid sequence from another polypeptide used as reference. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be naturally occurring such as an allelic variant, or it may be a variant that is not known to occur naturally, i.e. a variant may be artificially constructed.

In this text the phrases “percent sequence identity”, “percent identical with”, “percent similarity with” in the context of an amino acid sequence describes the number of matches of identical amino acids of two or more aligned amino acid sequences compared to the number of amino acid residues of the total length of the amino acid sequences. Thus, using an alignment, for two or more sequences the percentage of amino acid residues that are the same (such as e.g. 90%, or 95% or 100% identity over the full-length of the amino acid sequences) may be determined, when the sequences are compared and aligned for maximum correspondence as measured using a sequence comparison algorithm as known in the art, or in particular for short sequence motifs when manually aligned and visually inspected. The sequences which are compared to determine sequence identity may thus differ by substitution(s), addition(s) or deletion(s) of amino acids. Suitable programs for aligning protein sequences are known in the art.

Alignment of sequences for comparison may be conducted e.g. by the local homology algorithm of Smith and Waterman, Adv. Appl. Math. 2:482 (1981), by the global alignment algorithm of Needleman 25 and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, e.g. CLUSTALW, Clustal Omega, GAP, BESTFIT, BLAST, FASTA and TFASTA. Software for performing BLAST analyses is publicly available, e.g., through the National Center for Biotechnology-Information (NCBI BLAST algorithm (Altschul S F, et al (1997), Nucleic Acids Res. 25:3389-3402, http://blast.ncbi.nlm.nih.gov/). One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified 35 default parameters for protein and nucleic acid comparison, respectively.

In this text the term conservative amino acid substitution refers to modifications that are physically, biologically, chemically or functionally similar to the corresponding reference, e.g., similar size, shape, electric charge, chemical properties, including the ability to form covalent or hydrogen bonds, or the like.

For example, conservative amino acid substitutions include those in which the amino acid residue is replaced with another amino acid residue from the same side chain family, e.g., serine may be substituted for threonine. Amino acid residues are usually divided into families based on common, similar side-chain properties, such as:
1. nonpolar side chains (e.g., glycine, alanine, valine, leucine, isoleucine, methionine),
2. uncharged polar side chains (e.g., asparagine, glutamine, serine, threonine, tyrosine, proline, cysteine, tryptophan),
3. basic side chains (e.g., lysine, arginine, histidine, proline),
4. acidic side chains (e.g., aspartic acid, glutamic acid),
5. beta-branched side chains (e.g., threonine, valine, isoleucine), and
6. aromatic side chains (e.g., tyrosine, phenylalanine, tryptophan, histidine).

A conservative substitution may also involve the use of a non-natural amino acid.

In this text the term “similar protein sequences” are those which, when aligned, share similar amino acid residues and most often identical amino acid residues at corresponding positions of the sequences to be compared. Similar amino acid residues are grouped by chemical characteristics of the side chains into families. Said families are described above for “conservative amino acid substitutions”. The “percent similarity” between sequences is the number of positions that contain identical or similar residues at corresponding sequence positions of the sequences to be compared divided by the total number of positions compared and multiplied by 100%. For instance, if 6 out of 10 sequence positions have identical amino acid residues and 2 out of 10 positions contain similar residues, then the sequences have 80% similarity. The similarity between two sequences can, e.g., be determined using EMBOSS Needle.

In this text, the proximal region of an opsin CT is termed O-CT-proximal region and is defined to include the NR(K)Q motif (SEQ ID NO: 82) and the following approx. 10 amino acids, in particular 7 to 13, more particular 8 to 12 or most particular 9 to 11 amino acids in distal direction which often include a helix 8 (H8). The proximal region of an opsin CT optionally includes a palmitoylation site (C) at a distal end of H8.

In this text, the term truncated opsin CT refers to a truncated CT of the upstream opsin that is truncated at a truncation site beyond which in distal direction amino acids of the parent upstream opsin CT are excluded from the truncated opsin CT. The truncation site of the truncated CT is positioned at an amino acid positon at the distal end of the O-CT-proximal region or distal to it, in particular at the distal end of a distal extension to the O-CT-proximal region as defined above.

In preferred embodiments of the chimeric opsin GPCR protein, the truncation site of the truncated opsin CT is positioned at the distal end of the proximal region of the upstream opsin CT (O-CT-proximal region).

Yet, some embodiments of the chimeric opsin GPCR, comprise the truncation site of the upstream opsin CT at a distal end of a distal extension to the O-CT-proximal region. The distal extension to the O-CT-proximal region comprises a distal end at up to 5 or up to 10 or up to 16 or up to 22 or up to 28, 29, 30, 31, 32, 33, 34 or 35 amino acids downstream of the distal end of the O-CT-proximal region or in particular downstream of the palmitoylation site.

In some embodiments the truncation site of the opsin CT is selected at a position of up to 41 or up to 43 or up 45 or up to 47 amino acids downstream of the NR(K)Q motif (SEQ ID NO: 82).

In some preferred embodiments comprising the distal extension to the O-CT-proximal region, the upstream opsin is selected from the group of melanopsins.

In some embodiments comprising the distal extension to the O-CT-proximal region the upstream opsin comprises a long CT domain, e.g. it comprises a CT with at least 50, 65, 80, 100, 150 or 200 amino acids.

In some of these and other embodiments with the distal extension to the O-CT-proximal region, the distal end of the distal extension is selected such that subdomains of the upstream opsin CT which influence intracellular trafficking and kinetic properties that are specific to the upstream opsin are excluded.

The unusually long C-terminal cytoplasmic region of melanopsin (AA364-521 in murine OPN4) shows limited homology with other GPCRs. It may therefore contribute to the characteristic response properties of melanopsin, which sums the input over time being an environmental light detector entraining the circadian clock. It was suggested that AA381-397 of mouse Opn4, which are highly conserved between melanopsins of diverse species, have an important role in shaping the response of photoactivated melanopsin (Mure et al. 2016). Therefore, some embodiments of the chimeric opsin GPCR with melanopsin as upstream opsin comprise a truncated melanopsin at or distal to amino acid position 397 of mouse melanopsin to accelerate its response kinetics. Amino acid position 397 corresponds to 33 amino acids downstream of the palm site located at amino acid position 364 in mouse melanopsin.

Accordingly, some embodiments of the chimeric opsin GPCR protein, wherein the opsin is melanopsin, the truncated opsin CT includes up to approx. 44, amino acids downstream of the distal end of the NR(K)Q motif (SEQ ID NO: 82) corresponding to up to approx. 33, amino acids downstream of the palmitoylation site.

In some embodiments of the chimeric opsin GPCR the upstream opsin portion comprises the entire upstream opsin up to the truncation site, or the upstream opsin portion comprises a continuous region of the upstream opsin from the E(DRY) motif (SEQ ID NO: 80) up to the truncation site or, the upstream opsin portion comprises TM3, TM4, TM5, TM6 and TM7 and optionally the truncated upstream opsin CT up to the truncation site.

In some embodiments of the chimeric opsin GPCR protein, the upstream opsin portion comprises transmembrane domains TM3 and TM7, in particular comprises transmembrane domains TM3 to TM7, TM2 to TM7 or comprises TM1 to TM7.

In some embodiments of the chimeric opsin GPCR protein, the upstream opsin portion further comprises one or more of the extracellular domains selected from EL1, EL2, EL3 and the NT. In some embodiments of the chimeric opsin GPCR protein, the upstream opsin portion is derived from two or more parent opsins, in particular from two parent opsins.

In some embodiments, the upstream opsin portion comprises transmembrane domains derived from a parent opsin that is a non-human opsin and further comprises one or some, in particular two or three, or all extracellular domains derived from a parent opsin that is a human opsin. Advantageously, in these embodiments the human immune system does not recognize extracellular domains derived from a human opsin as foreign epitopes. Accordingly, in some preferred embodiments of the opsin GPCR protein, all extracellular domains are derived from a human opsin.

In some embodiments of the chimeric opsin GPCR protein, TM7 and the truncated opsin CT are derived from the same parent opsin.

In some embodiments of the chimeric opsin GPCR protein, the upstream opsin portion comprises all of the extracellular domains, all of the transmembrane domains and all intracellular loops.

In some embodiments of the chimeric opsin GPCR protein, the upstream opsin portion comprises the entire parent upstream opsin up to the truncation site of the CT.

In some embodiments of the chimeric opsin GPCR protein, the upstream opsin portion is derived from a mono-stable or from a bi-stable opsin or from a tri-stable opsin, in particular from a bi-stable opsin.

In some embodiments of the chimeric opsin GPCR protein, the upstream opsin portion is derived from a parent opsin selected from the group of opsins comprising:
melanopsin (OPN4)
rhodopsin (RHO)
cone opsins (OPN1SW, OPN1LW and OPN1MW)
jellyfish opsin (cubop, JellyOP)
jumping spider rhodopsin (JSR1)
Parapinopsin (PPO)
Neuropsin (OPN5)
Encephalopsin (OPN3)

In some embodiments of the chimeric opsin-GPCR protein, deletions or additions, in particular deletions or additions of up to 5 or up to 10 or up to 15 or up to 20 or up to 30 amino acids are present in the upstream opsin portion of the chimeric opsin GPCR compared to the physiological parent opsin. In some of these and other embodiments substitutions of amino acids, in particular conservative amino acid and/or in particular up to 5 or up to 10 or up to 15 or 20 substituted amino acids are present upstream opsin portion of the chimeric opsin GPCR compared to the physiological parent opsin.

In some embodiments of the chimeric opsin-GPCR protein, the amino acid sequence of the upstream opsin portion is at least 80%, 85%, 90%, 92%, 93%, 94%, 95%, 95%, 96%, 97%, 98%, 99%, in particular fully identical with or similar to the corresponding portion of the physiological one or more parent opsin.

In this text, the term target GPCR CT or target CT for short refers to the essentially complete CT of the target GPCR or to a functionally active variant thereof. Functionally active variants of the target CT are capable of coupling activation of the chimeric opsin GPCR or of the parent target GPCR into the specific signaling pathway of the parent GPCR with a similar efficiency.

The chimeric opsin GPCR according to one of the previous claim comprising a target GPCR comprising in particular a deletion of one or more amino acids, in particular an N terminal deletion between the NPxxY motif (SEQ ID NO: 81) and any amino acid position up to a palmitoylation site or up to an amino acid position proximally adjacent to the palmitoylation site.

In some embodiments the target GPCR CT is essentially complete. In particular an essentially complete target GPRC comprises a proximal end at or between the NPxxY (SEQ ID NO: 81) and the NR(K)Q (SEQ ID NO: 82) sites. In some embodiments the target GPCR CT has a proximal end one amino acid or up to 2 or up to 3 or up to 4 or up to 5 amino acids downstream of the NR(K)Q (SEQ ID NO: 82) site. In some embodiments the target CT has a proximal end between the NR(K)Q (SEQ ID NO: 82) siten and a distal end of a proximal region of the target CT at around 7 to 13 or 8 to 12 or 9 to 11 or approx. 10 amino acids distal to the NR(K)Q (SEQ ID NO: 82) site or in particular at a palmitoylation site or at an amino acid position corresponding to a palmitoylation site as defined above.

In some embodiments with a functional variant of the CT of the parent target GPCR one or more amino acids positioned between the NPxxY motif (SEQ ID NO: 81) at the distal end of TM7 of the target GPCR and the NR(K)Q motif (SEQ ID NO: 82) at the proximal end of H8 of the target GPCR CT are deleted or substituted.

In some further embodiments with a functional variant of the parent target GPCR a proximal region of the target GPCR is deleted up to the palmitoylation site at the distal end of H8. Thus, in these embodiments or in some further embodiments with target CTs not comprising an H8, the NR(K)Q motif (SEQ ID NO: 82) and subsequent 7 to 13 or 8 to 12 or 9 to 11 amino acids of the target GPCR are deleted. In particular embodiments, the amino acids proximal to the palmitoylation site or a site corresponding to a palmitoylation site as defined above are deleted.

In some of these embodiments such functional target GPCR CT and the truncated opsin CT are spliced together at the palmitoylation sites.

In some embodiments of a functional variant of the CT comprising substitutions and/or deletions a conserved NR(K)Q motif (SEQ ID NO: 82) is kept intact.

In some embodiments with a functional variant of the CT of the parent target GPCR, the amino acid sequence of the target GPCR CT of the target GPCR portion of the chimeric opsin GPCR protein is at least 85%, at least 90%, at least 92%, at least 93%, at least 94%, at least 95%, at least 95%, at least 96%, at least 97%, at least 98%, at least 99% identical or similar with the physiological parent target GPCR CT.

In some embodiments of the chimeric opsin GPCR, the target GPCR portion is derived from a non-opsin GPCR. In some other embodiments of the chimeric opsin GPCR, the target GPCR portion is derived from an opsin.

Thus, some embodiments comprise two opsins, an upstream opsin and a downstream opsin which is termed target opsin.

The upstream opsin portion is light-activatable and couples light-activation to the target GPCR CT. The target opsin, achieves coupling of light activation into the signaling cascade of the target opsin by binding to the Galpha protein physiologically pertaining to the target opsin.

In the truncated upstream opsin CT an O-CT-proximal region is embedded. Some embodiments comprising an upstream opsin and a target opsin, comprise two O-CT-proximal regions, one derived from the upstream opsin and one derived from the target GPCR. These embodiments may also comprise two opsin CT H8 subdomains, an upstream opsin H8 and a target GPCR H8.

In some embodiments of the chi-meric opsin GPCR protein, the target CT portion is derived from a parent target GPCR selected from the group of GPCR proteins comprising:

Class A GPCRs, in particular selected from the group comprising:
cone opsins, in particular OPN1SW, OPN1MW or OPN1LW,
serotonin receptors, in particular 5-HT7,
mu opioid receptor,
β-adrenergic receptor, in particular beta1-adrenoceptor, beta2-adrenoceptor and beta3-adrenoceptor;
   Class B GPCRs, in particular selected from the group comprising:
hormonal receptors, in particular glucagon receptor(GCGR)
   Class C GPCRs, in particular selected from the group comprising:
GABA_{B} receptors, in particular GABA_{B1} and GABA_{B2}
metabotropic glutamate receptors, in particular the mGluR6 and mGluR5 receptors.

In some embodiments of the chimeric opsin GPCR protein the target GPCR is a class A GPCR or a class B GPCR or a GPCR of another class except for a class C GPCR. In some of these embodiments, the target GPCR portion comprises one or more intracellular loops selected from IL1, IL2 and IL3.

In some embodiments of the chimeric opsin GPCR protein the target GPCR is a class C GPCR, in particular mGluR6. In some of these embodiments, the target class C GPCR portion, in particular the mGluR6 portion, comprises one or more intracellular loops selected from IL1, IL2 and IL3, with the proviso that one of the following criteria is fulfilled:
A: in the chimeric GPCR a concomitant presence of a naturally sized IL3 comprised in the upstream opsin portion and of a naturally sized IL2 of the class C GPCR at positions corresponding to their native position is excluded in order to avoid steric hindrance between these two ILs in the chimeric GPCR;
B: the upstream opsin portion comprises all of the intracellular loops IL1 to IL3;
C: the upstream opsin portion comprises IL1 and the target GPCR portion comprises both IL2 an IL3 which replace the upstream opsin IL2 and IL3 at corresponding positions.

In some embodiments of the chimeric opsin GPCR protein, the CT of the chimeric opsin GPCR further comprises a sequence element selected from the following group of elements:
Golgi export signal
Membrane trafficking sequence
sequence element encoding a fluorescent protein
   The one or more selected elements are arranged independently in any order at the C-terminal end of the CT of the chimeric opsin GPCR.

In some embodiments of the chi-meric opsin GPCR protein, the CT of the chimeric opsin GPCR comprises as a selected sequence element an export signal, in particular an endoplasmatic reticulum or a Golgi export signal, in particular the Golgi export signal from the potassium channel Kir2.1 with the amino acid sequence KSRITSEGEYIPLDQIDINV (SEQ ID NO 85) or from the ER export signal from Kir2.1 with the amino acid sequence FCYENEV(SEQ ID NO 86).

In some embodiments of the chi-meric opsin GPCR protein, the CT of the chimeric opsin GPCR comprises as a selected sequence element a membrane trafficking sequence, in particular from an opsin, more particularly the amino acid sequence ETSQVAPA (SEQ ID NO 53) that is also termed 1D4 epitope tag or 1D4 tag for short (cf. Gross et al. 2009; Lodowski et al. 2013).

In some embodiments of the chi-meric opsin GPCR protein, the CT of the chimeric opsin GPCR comprises a sequence element encoding a fluorescent protein, in particular selected from mKate2, TurboFP635 or mScarlet. In some of these embodiments the fluorescent protein is directly fused to the CT of the chimeric opsin GPCR and in some other of these embodiments the fluorescent protein is linked via an IRES or T2A sequence.

In some embodiments of the chi-meric opsin GPCR protein, the target GPCR portion further comprises IL1 and the IL1 of the target GPCR replaces the IL1 of the upstream opsin.

In some embodiments of the chi-meric opsin GPCR protein, IL3 of the upstream opsin is replaced by IL3 of the target GPCR. In some other embodiments a variable region within the upstream opsin IL3 of is replaced by IL3 of the target GPCR. Thereby a chimeric IL3 is obtained comprising the entire IL3 of the target GPCR at a position replacing a variable region within the opsin IL3 is formed. The portions of the upstream opsin IL3 that are proximally and distally adjacent to the variable region of the upstream opsin IL3 are retained in the chimeric IL3.

In some embodiments of the chi-meric opsin GPCR protein, in particular with mGluR6 as the target GPCR, the proximal end of the target CT is positioned at or upstream of the NR(K)Q motif (SEQ ID NO: 82) or at the palmitoylation site.

mGluR C-termini and their interaction with binding partners are well characterized (cf. e.g. Enz R, 2012). In mGluR6 the amino acids HPE constitute the NR(K)Q motif (SEQ ID NO: 82).

In some embodiments of the chi-meric opsin GPCR protein, the target GPCR is mGluR6 and IL3 of mGluR6 partially replaces a variable region of the opsin IL3 thereby forming a chimeric opsin-mGluR6 IL3. Accordingly, these embodiments comprise a chimeric IL3 in addition to the chimeric CT.

In some embodiments of the chi-meric opsin GPCR protein, the target GPCR is mGluR6 and the upstream opsin portion further comprises one or more of the intracellular loops selected from IL1, IL2 and IL3, with the proviso that a concomitant presence of a naturally sized IL3 comprised in the upstream opsin portion and a naturally sized IL2 comprised in mGluR6 portion in the opsin-mGluR6 chimeric protein is excluded.

In some embodiments of the chi-meric opsin GPCR protein, the upstream opsin portion is derived from melanopsin and comprises the NT, EL1 to EL3, TM1 to TM7, IL1 and the truncated opsin CT, and the target GPCR portion is derived either from mGluR6 or from hOPN1mw and comprises IL2, IL3 and the CT.

Some embodiments of the opsin-GPCR protein comprise or consist of an amino acid sequence selected from the group comprising SEQ ID NO 2, SEQ ID NO 4, SEQ ID NO 6, SEQ ID NO 8, SEQ ID NO 10 and SEQ ID NO 12, SEQ ID NO 14, SEQ ID NO 16, SEQ ID NO 18, SEQ ID NO 20, SEQ ID NO 22, SEQ ID NO 24, SEQ ID NO 26, SEQ ID NO 28, SEQ ID NO 30 and SEQ ID NO 32 SEQ ID NO 34, SEQ ID NO 36, SEQ ID NO 38, SEQ ID NO 40, SEQ ID NO 42 and SEQ ID NO 44. Some of these sequences comprise a C-terminally added sequence selected from a Golgi export signal and/or a 1D4 tag. Both the Golgi export signal and the 1D4 tag are optional. Accordingly, a sequence according to any of the above mentioned SEQ ID NO that comprises a Golgi export signal and/or a 1D4 tag is defined to include variants in which one or both of the optional C-terminally added sequence are absent.

Some particularly preferred embodiments of the chimeric opsin-GPCR protein comprise or consist of an amino acid sequence selected from the group comprising SEQ ID NO 18, SEQ ID NO 20, SEQ ID NO 22, SEQ ID NO 24, SEQ ID NO 26 and SEQ ID NO 28.

Some embodiments of the opsin-GPCR protein comprise an amino acid sequence which is a variant of any one of the sequences with the above mentioned SEQ ID No comprising one or more variation selected from
a conservative amino acid substitution,
a deletion in a range of 1 up to 3 or up to 5, up to 8 or up to 15 amino acids,
an insertion in a range of 1 up to 3, or up to 5, up to 8 or up to 15 amino acids, and
   wherein the chimeric opsin-GPCR protein exhibits a light activation dependent binding of the Galpha protein specific to the target GPCR. In some of these embodiments the target GPCR is mGluR6 and the chimeric opsin mGluR6 upon light activation binds Galpha(o).

Some of the embodiments of the opsin-GPCR protein comprising an amino acid sequence which is a variant of any one of the sequences with the above mentioned SEQ ID NO, said sequence has at least 85%, at least 90%, at least 92%, at least 93%, at least 94%, at least 95%, at least 96%, at least 97%, at least 98% or at least 99% identity.

In some preferred embodiments of the chimeric opsin GPCR, the target GPCR portion comprises or consists of an essentially complete CT with a proximal end between the NPxxy (SEQ ID NO: 81) and the NR(K)Q (SEQ ID NO: 82) site. In particularly preferred embodiments the target GPCR portion is derived from mGluR6 or from a second opsin (target opsin), in particular from a cone opsin or from rhodopsin.

In some of these and other preferred embodiments, the truncation site of the upstream opsin CT is positioned at the palmitoylation site or an amino acid position corresponding to a palmitoylation site as defined above. In some preferred embodiments the truncation site of the upstream opsin CT is positioned distal to the palmitoylation site e.g. up to 5 or up to 10 or up to 33 amino acids distal to the palmitoylation site.

In some of these and other preferred embodiments of the chimeric opsin GPCR, the upstream opsin portion comprises the entire upstream opsin up to the truncation site in the CT.

In some of these and other preferred embodiments, in particular when comprising an upstream opsin of non-human origin, optionally one or some or all extracellular domains are exchanged with extracellular domains of a human opsin. Optionally, in some of these preferred and other embodiments additionally or alternatively one or more of the intracellular loops selected from IL1, IL2 and IL3 is exchanged with an IL derived from the target GPCR, in particular IL1 or IL3 or a portion of IL3 as described above.

In some of these and other preferred embodiments of the chimeric opsin GPCR, the upstream opsin is selected from the group comprising melanopsin, a cone opsin, in particular middle wave cone opsin, box jelly fish opsin, or parapinopsin, or jumping spider rhodopsin (JSR1 or hJSR(S186F)).

Some of these and other preferred embodiments comprising melanopsin as upstream opsin and mGluR6 as target opsin and are termed mela-mGluR6 for short. In particularly preferred mela-mGluR6 embodiments that are termed mela(palm)-mGluR6, melanopsin it is truncated at its palmitoylation site at the distal end of H8 and is fused to an essentially complete CT of mGluR6. Some most preferred embodiments of the mela(palm)-mGluR6 chimera comprise or consist of an amino acid sequence selected from the group comprising SEQ ID NO 18, SEQ ID NO 20, SEQ ID NO 22, SEQ ID NO 24, SEQ ID NO 26 and SEQ ID NO 28 or a variant thereof.

As described above, the chimeric opsin GPCR protein of the present invention exhibits the common 3D structure of GPCR proteins. The chimeric opsin GPCR further comprises conserved motifs that are characteristic of all GPCR proteins such as the ionic lock with the E(D)RY motif (SEQ ID NO: 80), the NPxxY motif (SEQ ID NO: 81), the NR(K)Q motif (SEQ ID NO: 82). The chimeric GPCR protein according to the invention comprises an upstream opsin portion and a target GPCR portion.

The upstream opsin portion is also termed opsin portion for short, in embodiments, where the target GPCR is a non-opsin GPCR. The upstream opsin portion comprises the chromophore pocket and comprises the truncated opsin CT which comprises an O-CT-proximal region, optionally with a distal extension.

The O-CT-proximal region includes the NR(K)Q motif (SEQ ID NO: 82) and the following approx. 10 amino acids in distal direction which in many embodiments form an H8, optionally with one or more palmitoylation sites at its distal end. In some embodiments the O-CT-proximal region comprises a proximal extension up to the NPxxY (SEQ ID NO: 81) site and/or a distal extension up to approx. 33 amino acids downstream of a palmitoylation site corresponding to approx. up to 44 amino acids downstream of the NR(K)Q motif (SEQ ID NO: 82).

The target GPCR portion comprises essentially the entire CT of the target GPCR or a functional variant, in particular a functional fragment thereof. In some embodiments the target GPCR is a further opsin.

In some embodiments the chimeric opsin GPCR protein comprises two helices H8: one in the truncated opsin CT and one in the target GPCR CT.

It has been found that the O-CT-proximal region of the parent upstream opsin favorably enhances the conformational stability and light activation function of the upstream opsin portion in the chimeric opsin GPCR.

The target GPCR or a functional variant, in particular a functional fragment, thereof favorably mediates correct subcellular protein trafficking, including trafficking to the cell membrane. It is further thought, that the correct trafficking of the chimeric opsin GPCR protein to the cell membrane enhances both the response to light activation, as well as the shift of G protein binding selectivity from the Galpha protein of the parent opsin to the Galpha protein of the target GPCR.

Thus, it is the chimeric CT comprising an O-CT-proximal region and a target CT that leads to surprising technical effects: it alone suffices for transforming an upstream opsin such that it couples light-activation of the chimeric opsin GPCR into the endogenous signalosome of therapeutic target cells by binding to the Galpha protein of the target GPCR.

In some embodiments, the chimeric GPCR protein comprises an upstream opsin portion and an mGluR6 portion. The opsin portion comprises the chromophore pocket and the truncated opsin-CT including the NR(K)Q motif (SEQ ID NO: 82) and H8, and optionally one or more palmitoylation sites and/or optionally up to approx. 33 amino acids downstream of the palmitoylation site or up to approx. 44 amino acids downstream of the NR(K)Q motif (SEQ ID NO: 82). The mGluR6 portion comprises essentially the entire mGluR6-CT or a functional variant, in particular a functional fragment thereof. The opsin-mGluR6 chimeric GPCR protein according to these embodiments has a chimeric CT with the truncated opsin-CT upstream of the mGluR6-CT. In some embodiments, it comprises two helices H8: one in the truncated opsin CT and one in the target GPCR CT.

The chimeric CT suffices to target the exemplary chimeric opsin-mGluR6 protein to the signalosome of ON bipolar cells where upon light activation it binds to a Galpha(o) protein, instead of a different G protein of the signalosome that is endogenous in the physiological cellular environment of the parent melanopsin.

In some embodiments of the opsin mGluR6 chimera, the opsin is melanopsin.

Advantageously, exemplary embodiments of chimeric melanopsin GPCRs comprising a chimeric CT comprising a truncated melanopsin CT and a native target GPCR CT, exhibit a much faster response to light than the parent melanopsin. This is indeed desirable considering that response kinetics of melanopsin is adapted to its physiological role in regulation of circadian rhythm—a process requiring a slower kinetics of response to a change in light than the kinetics of response required for vision.

It was observed that the chimeric opsin GluR6 proteins couple light activation with a similar efficiency into the mGluR6 signaling cascade as their physiological parent opsins couple light activation into their signaling cascade in healthy photoreceptor cells.

Thus, the genetic transformation of physiologically light insensitive neurons, in particular ON bipolar cells, with nucleic acid molecules encoding the opsin-GluR6 chimeric GPCR bypasses photoreceptor cells in the visual signaling pathway and enables restoration of vision in retinas with degenerated photoreceptor cells by turning ON-bipolar cells into “replacement photoreceptors” that can activate the neural retina.

The prior art, e.g. above mentioned chimeric opsin mGluR6 proteins, has previously proven this therapeutic concept. The striking surprise of the present invention is that it suffices to simply equip a physiological opsin with a genetically engineered chimeric CT comprising the O-CT-proximal region, in particular embedded in the truncated opsin-CT and an essentially entire CT of the target GPCR. On top, this genetic design is applicable to all tested opsin GPCR chimera.

In contrast, the prior art teaches that besides the CT, the intracellular loops, in particular IL3 and also IL2 appeared to be particularly important or even required for G-protein selectivity (WO2012/174674, Kleinlogel, 2016; Tsai et al, 2018.

Thus, surprisingly, the specific G alpha(o) binding of the mGluR6 signalosome by the chimeric opsin-mGluR6 protein of the present invention is achieved in complete absence of any of the intracellular loops of mGluR6.

Indeed, exemplary chimeric opsin mGluR6 GPCR proteins comprising only the chimeric CT and no additional intracellular domains of mGluR6 mimic or surpass chimeric opsin GPCRs of the prior art comprising additionally replacement of intracellular loops in favorable properties such as e.g. fast kinetics and amplitude of the response to light activation, or such as correct intracellular trafficking to the subcellular compartment corresponding to the physiological compartment of cells comprising the parent target GPCR. For example, the chimeric opsin mGluR6 GPCRs of the present invention are more efficiently targeted to the dendrites in ON bipolar cells and enhance light-induced retinal responses compared to chimeric opto-mGluR6 available in the prior art (van Wyk et al, 2015).

A particular advantage of the chimeric opsin GPCR protein with the chimeric CT or the target opsin CT according to the invention is the great simplicity in the design, which requires minimal in silico modelling and genetic engineering based on the selection of a single mandatory splicing site, only. Nevertheless, the functional response to light activation of the chimeric opsin GPCR proteins, such as e.g. chimeric opsin mGluR6 or chimeric opsin 5-hydroxytryptamine receptor 7 (5-HT7), even correspond in magnitude and speed to the physiological response of the parent target GPCRs.

The first aspect of the invention further relates to a peptide comprising a chimeric C-terminal domain (chimeric CT) derived from a parent opsin CT and a parent target GPCR CT, in particular comprising a chimeric C-terminal domain (chimeric CT) of the chimeric opsin GPCR protein described above. The chimeric C-terminal peptide comprises a truncated C-terminal domain of an upstream opsin (truncated opsin-CT) including a proximal region of the CT (O-CT-proximal region). The O-CT-proximal region in particular comprises a helix 8 (H8) and a palmitoylation site corresponding to C322 or C323, respectively, of bovine rhodopsin. In some embodiments the O-CT-proximal region comprises a distal extension of e.g. up to approx. 33, 34 or 35 amino acids downstream of the palmitoylation site of the opsin. The peptide further comprises a C-terminal domain of a target GPCR (target GPCR CT) or a functional variant, in particular a functional fragment, thereof. The target GPCR CT is positioned downstream of the truncated opsin CT.

A second aspect of the invention relates to a nucleic acid molecule encoding the chimeric opsin GPCR protein and encoding the chimeric C-terminal peptide comprising the truncated opsin CT and the target GPCR CT or a functional variant thereof. Said nucleic acid molecule comprises or consists of a nucleic acid sequence encoding the chimeric opsin GPCR protein.

The chimeric opsin GPCR protein and the chimeric C-terminal peptide and the nucleic acid sequences encoding them are gene fusion products also termed gene splicing products, comprising fragments of a parent gene encoding a parent upstream opsin and fragments of a parent gene encoding a parent target GPCR as described above.

In this text, the nucleic acid sequence encoding the chimeric opsin GPCR or the chimeric C-terminal peptide is also termed chimeric opsin GPCR gene or chimeric opsin GPCR transgene.

In this text, the term transgene relates to a gene or a nucleic acid molecule transferred into the genome of an organism or cell. In particular, the term transgene refers to a gene or nucleic acid molecule encoding the chimeric opsin GPRCs or the chimeric C-terminal peptide of this invention.

In this text, the terms chimeric opsin GPCR protein and nucleic acid molecule encoding said opsin chimeric GPCR protein refer to a protein and a nucleic acid molecule that do not occur as such in nature. Rather they are artificial molecules obtainable by molecular techniques such as e.g. gene cloning, gene expression, recombinant nucleic acid technology, chemical synthesis such as e.g. solid phase chemical synthesis of recombinant nucleic acid molecules. They may be treated, fabricated and manipulated also in other ways as known in the art. For standard techniques used for molecular, genetic and biochemical methods and chemical methods see e.g. Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.

Thus, the second aspect of the invention relates to a nucleic acid molecule comprising or consisting of a nucleic acid sequence encoding the chimeric opsin GPCR or the peptide according to the first aspect of the invention.

In some embodiments of the nucleic acid molecule, it comprises a nucleic acid sequence encoding a chimeric opsin GPCR consisting of an amino acid sequence that is at least 90% identical to a sequence selected from a group of amino acid sequences comprising SEQ ID NO 2, SEQ ID NO 4, SEQ ID NO 6, SEQ ID NO 8, SEQ ID NO 10 and SEQ ID NO 12, SEQ ID NO 14, SEQ ID NO 16, SEQ ID NO 18, SEQ ID NO 20, SEQ ID NO 22, SEQ ID NO 24, SEQ ID NO 26, SEQ ID NO 28, SEQ ID NO 30 and SEQ ID NO 32 SEQ ID NO 34, SEQ ID NO 36, SEQ ID NO 38, SEQ ID NO 40, SEQ ID NO 42 and SEQ ID NO 44, such as at least 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to an amino acid sequence selected from SEQ ID NO 2, SEQ ID NO 4, SEQ ID NO 6, SEQ ID NO 8, SEQ ID NO 10 and SEQ ID NO 12, SEQ ID NO 14, SEQ ID NO 16, SEQ ID NO 18, SEQ ID NO 20, SEQ ID NO 22, SEQ ID NO 24, SEQ ID NO 26, SEQ ID NO 28, SEQ ID NO 30 and SEQ ID NO 32 SEQ ID NO 34, SEQ ID NO 36, SEQ ID NO 38, SEQ ID NO 40, SEQ ID NO 42 and SEQ ID NO 44.

Examples of such nucleic acid sequences encoding a chimeric opsin GPCR consisting of an amino acid sequence selected from SEQ ID NO 2, SEQ ID NO 4, SEQ ID NO 6, SEQ ID NO 8, SEQ ID NO 10 and SEQ ID NO 12, SEQ ID NO 14, SEQ ID NO 16, SEQ ID NO 18, SEQ ID NO 20, SEQ ID NO 22, SEQ ID NO 24, SEQ ID NO 26, SEQ ID NO 28, SEQ ID NO 30 and SEQ ID NO 32 SEQ ID NO 34, SEQ ID NO 36, SEQ ID NO 38, SEQ ID NO 40, SEQ ID NO 42 and SEQ ID NO 44. include, but are not limited to a nucleic acid sequence identical to a nucleic acid sequence selected from SEQ ID NO 1, SEQ ID NO 3, SEQ ID NO 5, SEQ ID NO 7, SEQ ID NO 9 and SEQ ID NO 11, SEQ ID NO 13, SEQ ID NO 15, SEQ ID NO 17, SEQ ID NO 19, SEQ ID NO 21, SEQ ID NO 23, SEQ ID NO 25, SEQ ID NO 27, SEQ ID NO 29 and SEQ ID NO 31 SEQ ID NO 33, SEQ ID NO 35, SEQ ID NO 37, SEQ ID NO 39, SEQ ID NO 41 and SEQ ID NO 43.

In some embodiments the nucleic acid molecule encoding the chimeric opsin GPCR comprises or consists of a nucleic acid sequence that is at least 70% identical to a nucleic acid sequence selected from the group comprising SEQ ID NO 1, SEQ ID NO 3, SEQ ID NO 5, SEQ ID NO 7, SEQ ID NO 9 and SEQ ID NO 11, SEQ ID NO 13, SEQ ID NO 15, SEQ ID NO 17, SEQ ID NO 19, SEQ ID NO 21, SEQ ID NO 23, SEQ ID NO 25, SEQ ID NO 27, SEQ ID NO 29 and SEQ ID NO 31 SEQ ID NO 33, SEQ ID NO 35, SEQ ID NO 37, SEQ ID NO 39, SEQ ID NO 41 and SEQ ID NO 43; such as at least 75%, 80%, 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to a nucleic acid sequence selected from the group comprising SEQ ID NO 1, SEQ ID NO 3, SEQ ID NO 5, SEQ ID NO 7, SEQ ID NO 9 and SEQ ID NO 11, SEQ ID NO 13, SEQ ID NO 15, SEQ ID NO 17, SEQ ID NO 19, SEQ ID NO 21, SEQ ID NO 23, SEQ ID NO 25, SEQ ID NO 27, SEQ ID NO 29 and SEQ ID NO 31 SEQ ID NO 33, SEQ ID NO 35, SEQ ID NO 37, SEQ ID NO 39, SEQ ID NO 41 and SEQ ID NO 43.

In some particularly preferred embodiment of the nucleic acid molecule according to claim 38, encoding a preferred mela(palm mGluR6 chimeric opsin GPCR, wherein said nucleic acid molecule comprises or consists of an nucleic acid sequence selected from the group comprising SEQ ID NO 17, SEQ ID NO 19, SEQ ID NO 21, SEQ ID NO 23, SEQ ID NO 25 and SEQ ID NO 27.

A third aspect of the invention relates to an adeno-associated virus capsid (AAV capsid) polypeptide for medical use of delivering a nucleic acid molecule according to the second aspect of the invention into a target cell. Thus, the third aspect provides an AAV capsid for the transfer of a transgene encoding the chimeric opsin GPCR protein according to the first aspect into a target cell. The third aspect also relates to a nucleic acid molecule comprising a sequence encoding the AAV capsid polypeptide for said medical use.

A second invention that is independent of the main invention described herein in an aspect A relates to a novel AAV capsid polypeptide per se and in an aspect B to a nucleic acid molecule comprising a sequence encoding said novel AAV capsid.

An aspect C of the independent, second invention relates to the novel AAV capsid polypeptide—and the nucleic acid molecule encoding it—for medical use of delivering a transgene to a target cell. Aspect C of the independent, second invention also includes the novel AAV capsid polypeptide—and the nucleic acid molecule encoding it—for medical use of delivering the nucleic acid molecules encoding the chimeric opsin GPCR according to the second and first aspect, respectively, of the first, main invention to a target cell.

An aspect D of the independent, second invention relates to a recombinant AAV vector comprising the nucleic acid molecule according to aspect B encoding the novel capsid according to aspect A of the independent second invention.

In the description and the claims below the term AAV capsid or capsid refers to both the capsid for medical use according to the main invention and the capsid per se according to the independent second invention, unless differently apparent from the context.

Accordingly, in the third aspect of the invention an adeno-associated virus (AAV) capsid polypeptide and a nucleic acid molecule encoding the AAV capsid polypeptide are provided for use in medical therapy to deliver a nucleic acid molecule according to the second aspect of the invention encoding the chimeric opsin GPCR protein according to the first aspect of the invention to a target cell.

In some embodiments the capsid is a capsid protein of AAV2, AAV2(7m8) (Dalkara D et al, 2013) or AAV8(BP2) (Cronin et al, 2014) or a variant derived thereof.

In wild-type AAV the genome includes the Cap gene which encodes the capsid proteins VP1, VP2 and VP3, which interact together to form a capsid of an cosahedral symmetry, and the assembly-activating protein (AAP), which is required for stabilizing and transporting newly produced VP proteins from the cytoplasm into the cell nucleus. All three VPs are translated from one mRNA and spliced differently. The largest 90 kDa VP1 is an unspliced transcript, the 72 kDa VP2 is translated from a non-conventional ACG start codon whereas the smallest 60 kDa VP3 is translated from an AUG codon. All the three VPs have overlapping C-termini.

A reference to an amino acid position in an AAV capsid in the context of the present text relates to the amino acid sequence of the capsid protein VP1 of AAV2 according to the reference sequence of AAV2 which is accessible at GenBank entry No. J01901.1 (Adeno-associated virus 2, complete genome).

In some embodiments the AAV capsid comprises a peptide insert. In some embodiments, the peptide is inserted in particular at a peak or spiky protrusion.

The peptide is inserted at position 587 of the AAV2 capsid. Spiky protrusions (peaks) represent the most exposed regions of the capsids. The highest peak is located at amino acid position 453 and second highest at position 587 on the AAV2 capsid. These peaks accept peptide insertions without disturbing capsid assembly and provide opportunities for targeting non-permissive cells. Likewise, protrusions represent critical sites of AAVs host interaction, receptor binding and immunogenicity.

The wild type capsid AAV2 sequence (SEQ ID NO 59) is shown below with the insertion point of the above mentioned peptides between N587 and R588 that are marked in bold and underlined).

| | |
|---|---|
| MAADGYLPDW LEDTLSEGIR QWWKLKPGPP PPKPAERHKD DSRGLVLPGY KYLGPENGLD | 60 |
| KGEPVNEADA AALEHDKAYD RQLDSGDNPY LKYNHADAEF QERLKEDTSF GGNLGRAVFQ | 120 |
| AKKRVLEPLG LVEEPVKTAP GKKRPVEHSP VEPDSSSGTG KAGQQPARKR LNFGQTGDAD | 180 |
| SVPDPQPLGQ PPAAPSGLGT NTMATGSGAP MADNNEGADG VGNSSGNWHC DSTWMGDRVI | 240 |
| TTSTRTWALP TYNNHLYKQI SSQSGASNDN HYFGYSTPWG YFDENRFHCH FSPRDWQRLI | 300 |
| NNNWGFRPKR LNFKLFNIQV KEVTQNDGTT TIANNLTSTV QVFTDSEYQL PYVLGSAHQG | 360 |
| CLPPFPADVF MVPQYGYLTL NNGSQAVGRS SFYCLEYFPS QMLRTGNNFT FSYTFEDVPF | 420 |
| HSSYAHSQSL DRLMNPLIDQ YLYYLSRTNT PSGTTTQSRL QFSQAGASDI RDQSRNWLPG | 480 |
| PCYRQQRVSK TSADNNNSEF SWTGATKYHL NGRDSLVNPG PAMASHKDDE EKFFPQSGVL | 540 |
| IFGKQGSEKT NVDIEKVMIT DEEEIRTTNP VATEQYGSVS TNLQRGNRQA ATADVNTQGV | 600 |
| LPGMVWQDRD VYLQGPIWAK IPHTDGHFHP SPLMGGFGLK HPPPQILIKN TPVPANPSTT | 660 |
| FSAAKFASFI TQYSTGQVSV EIEWELQKEN SKRWNPEIQY TSNYNKSVNV DFTVDTNGVY | 720 |
| SEPRPIGTRY LTRNL | |

In some embodiments, the AAV capsid protein is an AAV2 capsid protein and comprises an amino acid insert between amino acids 587 and 588, wherein the peptide insert is selected from the group of peptides comprising

(SEQ ID NO 60)
SASEAST
(SEQ ID NO 61)
TPPSITA
(SEQ ID NO 62)
PRTPHTA
(SEQ ID NO 63)
NHAPNHC

Peptide inserts at between N587 and R588 have been described in the prior art (David A., 2018; EP application No. 19206603.3, unpublished.) In these embodiments the AAV capsid polypeptide comprises a peptide insert which consists of 7 amino acids, also called a 7-mer peptide insert for short.

In some further embodiments the AAV capsid polypeptide comprises a peptide insert which is a 7 to 13-mer. In particular, these embodiments comprise a 7-mer, such as the 7-mer peptide insert described above and additionally comprise one or two flanking linkers of 0-6 amino acids with 6 being the maximum number of the total number of N- and C-terminally added flanking amino acids.

Some exemplary embodiments comprise of peptide inserts comprising no linker sequence, other embodiments comprise a linker on one side or on both sides. In some embodiments the linker is selected from but not limited to a group of amino acids comprising alanine (A), Asparagine (N), Lysine (L), Arginine (R), Threonine (T) or glycine (G) or a mixture thereof.

In some preferred embodiments the one or two flanking linker comprise or preferably consist of amino acids selected from
i. amino acids G and A or
ii. amino acids A, N, L, T, R, G, A, N, L and R, in particular A, L, N, R.

In some preferred embodiments, one or both of the flanking linkers comprise at least one amino acid selected from N and R. In some particularly preferred embodiments, the linkers comprise 2 or 3 amino acids on either side. In some of these and other embodiments the linker consists of one or more amino acid selected from the amino acids A, L, N, R. In exemplary preferred embodiments the linker and peptide insert are in the configuration of NLA-peptide-AR

In some preferred embodiments the capsid is an AAV2 capsid protein or a mutant variant thereof that comprises a peptide insert between N587 and R588, selected from

(SEQ ID NO 64)
AAASASEASTAA,
(SEQ ID NO 65)
AAATPPSITAAA,
(SEQ ID NO 66)
AAAPRTPHTAAA,
(SEQ ID NO 67)
NLANHAPNHCAR,
(SEQ ID NO 68)
NLAPRTPHTAAR.

In this text embodiments of AAV2 capsid comprising an insert are also referred to in an abbreviated nomenclature listing the wildtype AAV serotype followed by the modification in parenthesis. Such abbreviated nomenclature for exemplary capsids comprising a peptide insert as described above is e.g. an AAV2(NHAPNHC) (“NHAPNHC” is disclosed as SEQ ID NO: 63) referring to an AAV2 capsid comprising the 7 amino acids in the peptide insert listed in parenthesis and optionally further comprising one or two flanking linkers or e.g. an AAV2(PRTPHTA) (“PRTPHTA” is disclosed as SEQ ID NO: 62) capsid comprising a peptide insert with the listed 7 amino acids PRTPHTA (SEQ ID NO: 62) and optionally including one or two flanking linkers.

The above mentioned exemplary embodiments of peptide inserts comprising flanking linkers are listed below with the flanking linkers in a slightly expanded sequence context of the AAV2 capsid. The exemplary embodiments of the linkers are underlined. The first three linkers comprise alanine and are in the form of -AAA- . . . -AA- and the last two linkers comprise alternative amino acids, i.e. arginine (R), asparagine (N) and lysine (L) in addition to alanine (A) are in the form of -NLA- . . . -AR- as shown below:

SEQ ID NO 69:
580-VSTNLQRGN-AAA-SASEAST-AA-RQAATADVNTQGVLPG
SEQ ID NO 70:
580-VSTNLQRGN-AAA-TPPSITA-AA-RQAATADVNTQGVLPG
SEQ ID NO 71:
580-VSTNLQRGN-AAA-PRTPHTA-AA-RQAATADVNTQGVLPG
SEQ ID NO 72:
580-VSTNLQRGN-NLA-NHAPNHC-AR-RQAATADVNTQGVLPG
SEQ ID NO 73:
580-VSTNLQRGN-NLA-PRTPHTA-AR-RQAATADVNTQGVLPG

Capsids comprising the peptide insert according to SEQ ID NO 67, NLANHAPNHCAR, or according to SEQ ID NO 68, NLAPRTPHTAAR, are novel per se and constitute an independent invention. Thus, these novel capsids are not limited to the context of rAAV2 vectors for packaging the transgene encoding the chimeric opsin GPCR proteins described herein. In particular, these novel capsids are not limited to medical use with the chimeric opsin GPCR or the nucleic acid molecule encoding it according to the first and second aspect of the present invention.

Thus, in an independent invention an adeno-associated virus (AAV) capsid polypeptide is provided which comprises a peptide insert at a position between 587 to 592, preferably between N587 and R588 of the AAV serotype 2 capsid or at a position homologous thereto in an AAV of another serotype, wherein the peptide insert is selected from the group of sequences comprising:

(SEQ ID NO 67)
NLANHAPNHCAR,
(SEQ ID NO 68)
NLAPRTPHTAAR

In some embodiments, the capsid protein is an AAV2 capsid and comprises at least one mutation, wherein this at least one mutation is selected from:
a. a tyrosine (Y) to phenylalanine (F) at amino acid position 252, 272, 444, 500, 700, 704 and/or 730; and/or
b. a threonine (T) to valine (V) at amino acid position 491.

A particularly preferred embodiment of the novel capsid comprises the amino acid sequences of AAV2 capsid protein with the NLAPRTPHTAAR (SEQ ID NO: 68) insertion according to SEQ ID NO 74 as shown below:

VP3 (grey sequence) overlapping with VP1, tyrosine to phenylalanine (Y-F) mutations are highlighted in dark grey and underlined, amino acid numbering refers to the whole VP1 sequence. The highest peak at G453 and the second highest peak at N587, where the motif was inserted, is underlined and indicated by a white underlay. The insertion is in italics and boxed.

(SEQ ID NO: 74)
MAADGYLPDWLEDTLSEGIRQWWKLKPGPPPPKPAERHKDDSR
GLVLPGYKYLGPENGLDKGEPVNEADAAALEHDKAYDRQLDSG
DNPYLKYNHADAEFQERLKEDTSFGGNLGRAVFQAKKRVLEPL
GLVEEPVKTAPGKKRPVEHSPVEPDSSSGTGKAGQQPARKRLN
FGQTGDADSVPDPQPLGQPPAAPSGLGTNTMATGSGAPMADNN
EGADGVGNSSGNWHCDSTWMGDRVITTSTRTWALPTFNNHLYK
QISSQSGASNDNHFFGYSTPWGYFDENRFHCHESPRDWQRLIN
NNWGFRPKRLNFKLFNIQVKEVTQNDGTTTIANNLTSTVQVFT
DSEYQLPYVLGSAHQGCLPPFPADVEMVPQYGYLTLNNGSQAV
GRSSFYCLEYFPSQMLRTGNNFTFSYTFEDVPFHSSYAHSQSL
DRLMNPLIDQYLYFLSRTNTPSGTTTQSRLQFSQAGASDIRDQ
SRNWLPGPCYRQQRVSKTSADNNNSEFSWTGATKYHLNGRDSL
VNPGPAMASHKDDEEKFFPQSGVLIFGKQGSEKTNVDIEKVMI

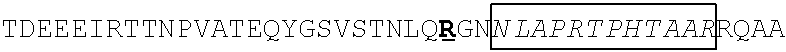

TADVNTQGVLPGMVWQDRDVYLQGPIWAKIPHTDGHFHPSPLM
GGFGLKHPPPQILIKNTPVPANPSTTFSAAKFASFITQYSTGQ
VSVEIEWELQKENSKRWNPEIQFTSNYNKSVNVDFTVDINGVY
SEPRPIGTRFLTRNL

The third aspect and the independent invention also relate to nucleic acid molecules encoding the AAV capsids as described above.

In some embodiments, the nucleic acid molecule comprises or consists of a nucleic acid sequence encoding a capsid polypeptide selected from AAV2, AAV2(7m8) or AAV8(BP2) or AAV2(NHAPNHC) (“NHAPNHC” is disclosed as SEQ ID NO: 63) or AAV2(PRTPHTA) (“PRTPHTA” is disclosed as SEQ ID NO: 62).

In some embodiments, The nucleic acid molecule according to claim 49, wherein the nucleic acid molecule comprises or consists of a nucleic acid sequence encoding a capsid polypeptide comprising an amino acid sequence with a peptide insert between N587 and R588 of the AAV2 genome, selected from

(SEQ ID NO 64)
AAASASEASTAA,
(SEQ ID NO 65)
AAATPPSITAAA,
(SEQ ID NO 66)
AAAPRTPHTAAA,
(SEQ ID NO 67)
NLANHAPNHCAR,
(SEQ ID NO 68)
NLAPRTPHTAAR.

In some embodiments, the nucleic acid molecule comprises a transgene in particular encoding a chimeric opsin GPCR. In some preferred of these embodiments the transgene comprises or consists of a nucleic acid sequence selected from the group comprising SEQ ID NO 1, SEQ ID NO 3, SEQ ID NO 5, SEQ ID NO 7, SEQ ID NO 9 and SEQ ID NO 11, SEQ ID NO 13, SEQ ID NO 15, SEQ ID NO 17, SEQ ID NO 19, SEQ ID NO 21, SEQ ID NO 23, SEQ ID NO 25, SEQ ID NO 27, SEQ ID NO 29 and SEQ ID NO 31 SEQ ID NO 33, SEQ ID NO 35, SEQ ID NO 37, SEQ ID NO 39, SEQ ID NO 41 and SEQ ID NO 43.

In some particularly preferred embodiments, the nucleic acid molecule comprises a transgene encoding a mela(palm)-mGluR6 chimeric GPCR comprising or consisting of a nucleic acid sequence selected from the group comprising SEQ ID NO 17, SEQ ID NO 19, SEQ ID NO 21, SEQ ID NO 23, SEQ ID NO 25 and SEQ ID NO 27. In some embodiments of the nucleic acid molecule encoding the capsid and a transgene, the transgene is operably linked to a cell specific promoter. In some of these embodiments the cell specific promoter is in particular an ON bipolar cell specific promoter, more particularly a promoter selected from the group comprising a 200En-mGluR500P promoter, a 770En_454P(hGRM6) promoter according to SEQ ID NO 75 or a 444En_454P(hGRM6) promoter according to SEQ ID NO 76 or an endogenous mGluR6 promoter of retinal ON-bipolar cells or elements thereof.

The terms Mela(palm)-mGluR6 or Mela(palm+33)-mGluR6 refer to preferred embodiments of melanopsin mGluR6 chimeric opsin GPCRs comprising a truncated melanopsin CT truncated at the palmitoylation site or truncated at 33 amino acids downstream of the palmitoylation site, respectively.

Accordingly, in some particularly preferred embodiments of the nucleic acid molecule encoding the capsid, the capsid is selected from an AAV2(7m8) or AAV8(BP2) or AAV2(NHAPNHC) (“NHAPNHC” is disclosed as SEQ ID NO: 63) or AAV2(PRTPHTA) (“PRTPHTA” is disclosed as SEQ ID NO: 62) capsid. Additionally, said nucleic acid molecules further comprise a transgene encoding a preferred embodiment of the chimeric opsin GPCR such as Mela(palm)-mGluR6 or Mela(palm+33)-mGluR6 and furthermore the transgene is under control of the 770En-445P(hGRM6) promoter or the 444En_454P(hGRM6) promoter.

In some embodiments of the independent invention relating to the novel rAAV capsid, a vector is provided comprising the novel rAVV capsid with the novel peptide insert as described above.

Whenever an embodiment of the capsid according to the fifth aspect of the invention refers to a specific sequence according to a particular SEQ ID NO, it is understood that variants of the specific sequence as described above are included in these embodiments.

A fourth aspect of the invention relates to a vector comprising a nucleic acid molecule according to the second aspect of the invention encoding the chimeric opsin GPCR protein or the chimeric C-terminal peptide according to the first aspect of the invention. Thus, the fourth aspect relates to a vector for gene transfer into a target cell and in particular also for expressing the chimeric opsin GPCR therein. In other words the vector according to the fourth aspect of the invention comprises a transgene encoding the chimeric opsin GPRCs or the chimeric C-terminal peptide according to the first aspect of the invention.

Accordingly, a vector in particular a nucleic acid expression vector is provided comprising a nucleic acid encoding the chimeric opsin GPCR protein or the chimeric C-terminal peptide encoded by the nucleic acid molecule as described in the first and second aspect of the invention, respectively. The nucleic expression vector comprises a promoter operably linked to a transgene encoded by the nucleid acid molecule encoding the chimeric opsin GPCR.

In some embodiments, the transgene is preceded by an optimized KOZAK sequence. The KOZAK sequence has the consensus (gcc)gccAccAUGG (SEQ ID NO 77) or (gcc)gccGccAUGG (SEQ ID NO 78) and enhances the initiation of the translation.

In some embodiments, the nucleic acid expression vector also comprises a WPRE (Woodchuck hepatitis virus post-transcriptional regulatory element) regulatory sequence (cf. SEQ ID NO 20 in Hulliger et al. 2010). The WPRE is a DNA sequence that, when transcribed, creates a tertiary structure enhancing expression.

In some embodiments, the nucleic acid expression vector also comprises a polyA tail, which is inserted downstream of the transgene. The polyA tail promotes translation of the transgene.

In some embodiments, the vector is derived from an adeno-associated virus (AAV). Said vector is a recombinant (rAAV) vector as it comprises the nucleic acid molecule encoding the chimeric opsin GPCR protein or the chimeric C-terminal peptide according to the first aspect of the invention described above.

In some embodiments, the rAAV vector is either a single-stranded vector (ssAAV) or a self-complementary vector (scAAV).

In some embodiments, the vector is a recombinant AAV vector, in particular selected from the group of AAV serotypes comprising AAV1, AAV2, AAV3, AAV4, AAV5, AAV6, AAV7, AAV8, AAV9, AAV10, AAV11 or AAV12. In some preferred embodiments, the vector is a rAAV2 or a rAAV8 vector.

In some of these and other embodiments the vector further comprises a nucleic acid sequence selected from the group of sequences comprising:
a sequence encoding an AAV capsid protein, and/or
a promoter, in particular a cell-specific promoter, more particularly a bipolar cell specific promoter.

In some embodiments comprising a promoter, in particular a cell-specific promoter, the vector further comprises an enhancer sequence and optionally a spacer. From 5′-end to 3′-end, the vector comprises first the enhancer, then the optional spacer and then the promoter. The transgene is located in 3′-direction of the promoter for expression of the transgene driven by the promoter, i.e. the transgene is operably linked to the promoter.

In some embodiments, in particular of the vector expressing a nucleic acid molecule encoding a chimeric opsin GPCR comprising an mGluR6 target GPCR CT, the vector comprises an ON-bipolar cell specific promoter. In some of these embodiments the ON-bipolar cell specific promoter is selected from the group comprising a GRM6-sv40 promoter (Kim et al, 2008) or a 4×GRM6-sv40 promoter (Cronin et al., 2014) or a 200En-mGluR500P promoter (Lu et al., 2016) or a 770En_454P(hGRM6) or 444En_454P(hGRM6) promoter (cf. Hulliger et al., 2020 and EP19200082.6 (unpublished)). The 770En_454P(hGRM6) promoter comprises or consists of SEQ ID NO 75. The 770En_454P(hGRM6) promoter comprises the enhancer 770En(hGRM6) (−14236 to −13467 rel. TLSS GRM6) containing the 300 bp conserved sequence between the murine and human genomes (−13873 to −13467 rel. TLSS GRM6) and in addition containing the 3′ ChIP-seq peaks and Dnase hypersensibility cluster (−13990 to −13816 rel. TLSS GRM6).

The 444En_454P(hGRM6) promoter comprises or consists of SEQ ID NO 76. The 444En_454P(hGRM6) promoter comprises the enhancer 444En(hGRM6) (−14033 to −13590 rel. TLSS GRM6) and is a 3′ and 5′ truncated version of the 770En(hGRM6) including 3′ and 5′ only the ChiP-seq peaks.

In some embodiments of the vector comprising a cell specific promoter, the cell specific promoter is an endogenous mGluR6 promoter of retinal ON-bipolar cells or elements thereof.

Some preferred embodiments of the vector comprising an ON-bipolar cell specific promoter as described above express a nucleic acid molecule encoding a chimeric melanopsin-mGluR6. In some of these embodiments, the chimeric melanopsin mGluR6 protein comprises an opsin CT truncated at the palmitoylation site, also termed Mela(palm)-mGluR6 for short, or truncated 33 amino acids downstream of the palmitoylation site also termed Mela(palm+33)-mGluR6 for short. In further preferred embodiments the vector comprises a chimeric OPN1mw-mGluR6 or a chimeric opsin GPCR comprising two opsins.

Some particularly preferred embodiments of the vector express Mela(palm)-mGluR6 according to one of SEQ ID NO 17, SEQ ID NO 19, SEQ ID NO 21, SEQ ID NO 23, SEQ ID NO 25 and SEQ ID NO 27 or Mela(palm+33)-mGluR6, in particular according Seq ID NO 15 or a Mela-mGluR6 additionally comprising an intracellular loop, in particular according to a sequence selected from SEQ ID NO 29 or SEQ ID NO 31 under the control of an ON-bipolar cell specific promoter, selected in particular from 200En-mGluR500P, 770En_454P(hGRM6) or 444En_454P(hGRM6) promoter or the endogenous mGluR6 promoter of retinal ON-bipolar cells or elements thereof.

Some embodiments of the vector comprise a nucleic acid sequence encoding the AAV capsid according to the third aspect of the invention. In some preferred embodiments the vector encodes the AAV capsid with a peptide insert between N587 and R588 as described above.

In some preferred embodiments the vector comprises the 770En-445P(hGRM6) promoter operably linked to a transgene encoding the chimeric opsin GPCR, and further comprises a nucleic acid molecule expressing AAV2(7m8) or AAV8(BP2) or AAV2(NHAPNHC) (“NHAPNHC” is disclosed as SEQ ID NO: 63) or AAV2(PRTPHTA) (“PRTPHTA” is disclosed as SEQ ID NO: 62). In some of these and other embodiments the chimeric opsin GPCR is preferably selected from
a chimeric opsin GPCR comprising melanopsin or hOPN1mw as upstream opsin and mGluR6 as target opsin or a chimeric opsin GPCR comprising two opsins,
a chimeric opsin GPCR selected from Mela(palm)-mGluR6 or Mela(palm+33)-mGluR6
a chimeric opsin GPCR according to a nucleic acid sequence selected from the group comprising SEQ ID NO 15, SEQ ID NO 17, SEQ ID NO 19, SEQ ID NO 21, SEQ ID NO 23, SEQ ID NO 25 and SEQ ID NO 27 SEQ ID NO 29 or SEQ ID NO 31.

A particularly preferred embodiment of the vector comprises or consists of the sequence according to SEQ ID NO 79. In this embodiment of the vector it comprises the exemplary hmela(palm)-mGluR6 transgene according to SEQ ID NO 19 under the control of the 770En-445P(hGRM6)promoter and further comprises a nucleic acid sequence encoding the AAV8(BP2) capsid.

Whenever an embodiment of the vector according to the fourth aspect of the invention refers to a specific sequence according to a particular SEQ ID NO, it is understood that variants of the specific sequence as described above are included in these embodiments.

A fifth aspect of the invention relates to carriers such as particles, in particular nano particles, vesicles, cell lines—in particular excluding germ cell lines—and animals comprising or expressing nucleic acid molecules according to the second aspect, or vectors according to the third aspect or comprising the chimeric opsin GPCR according to the first aspect.

In some embodiments of the fifth aspect of the invention a transgenic animal, in particular a transgenic mouse or a transgenic cell line is provided. The transgenic animal or the transgenic cell line comprises the nucleic acid molecule of the second aspect of the invention or the vector of the fourth aspect and/or it expresses the chimeric opsin GPCR protein according to the first aspect of the invention.

Some embodiments of the transgenic cells are derived from a suitable cell line for expressing the chimeric opsin GPCR protein such as a stem cell line which optionally excludes transgenic germ cell lines or an organotypic cell line. In particular, a suitable cell line is selected from the group of cell lines comprising
HEK293-GIRK cells,
inner retinal neurons, in particular ON bipolar cells,
kidney cells and
cells expressing a G protein selected from Gs, Gq or G_{12/13}.

Some embodiments of the transgenic animal or the transgenic cell comprise a CRISPR/cas modified genome. CRISPR/Cas genome editing is known to the skilled person (see e.g. Vandemoortele et al. (2017), e.g. Long et al. (2018), e.g. Hsu et al (2014), Cell 157(6):1262-1278 and references therein).

In some embodiments of the fifth aspect, the invention provides a carrier, in particular a particle or a nanoparticle or a vesicle, for transfer of the chimeric opsin GPCR or the nucleic acid molecule or vector encoding it according to one of the previous aspects of the invention to a target cell.

In some embodiments the carrier comprises a nucleic acid molecule according to the second aspect or a vector according to the fourth aspect comprising the transgene encoding the chimeric opsin GPCR according to the first aspect of the invention or it comprises the chimeric opsin GPCR protein according to the first aspect of the invention. In some embodiments, the carrier is a nano- or a micro-particle, that is in particular suitable for use with a gene gun. In some of these and other embodiments the carrier is a gold particle.

In this text, the carrier for transfer of the chimeric opsin GPCR protein or the nucleic acid molecule or vector, is also referred to shorter as “carrier for transfer”. The carrier for transfer refers to any suitable chemical or physical structure that is capable of attaching or packaging the chimeric opsin-GPCR protein or nucleic acid molecule or vector comprising the transgene encoding the chimeric opsin GPCR suitable for its transfer to a recipient genome of a target cell or target organism of a human or non-human animal.

Exemplary embodiments of the carrier for transfer are vesicles and particles, in particular micro- or nano-particles. Exemplary vesicles include e.g. membrane vesicles of biological or synthetic origin. Exemplary particles, are in particular micro- and nano-particles that are suitable for use with a gene gun and include e.g. gold particles coated with the chimeric opsin GPCR protein or the chimeric nucleic acid encoding it, in particular as an adsorbed or as a covalently attached ligand (O'Brian and Lummis, 2011). In some embodiments of the carrier it comprises the transgene and a CRISPR/cas cassette, i.e. a plasmid encoding a Cas enzyme such as e.g. Cas9 and one or more guide RNA(s) (gRNA), particularly single guide RNA(s) (sgRNA), or a plasmid encoding a Cas enzyme, in particular Cas9, to be combined with a separate transfection of one or more gRNAs, particularly sgRNAs.

In some of these and other embodiments of the invention, the carrier comprises a nucleic acid sequence according to the second aspect of the invention or a vector according to the fourth aspect of the invention comprising the transgene and a CRISPR/cas cassette.

In some preferred embodiments, the transgenic animal or the transgenic cell or the carrier for transfer described above comprise the transgene encoding a chimeric melnaopsin-mGluR6 (Mela-mGluR6), in particular Mela(palm)-mGluR6 or Mela(palm+33)-mGluR6 or a chimeric OPN1mw-mGluR6 or a chimeric opsin GPCR comprising two opsins.

In some further preferred embodiments, the transgenic animal or the transgenic cell or the carrier for transfer described above, the transgene encodes a chimeric Mela-mGluR6 selected from the group comprising:
a Mela(palm)-mGluR6, in particular according to one of the sequences selected from the group comprising SEQ ID NO 17, SEQ ID NO 19, SEQ ID NO 21, SEQ ID NO 23, SEQ ID NO 25 and SEQ ID NO 27 or
a Mela(palm+33)-mGluR6, in particular according to SEQ ID NO 15 or
a Mela-mGluR6 additionally comprising an intracellular loop, in particular according to a sequence selected from SEQ ID NO 29 or SEQ ID NO 31.

Whenever an embodiment of the carriers, cells or animals according to the fifth aspect of the invention refers to a specific sequence according to a particular SEQ ID NO, it is understood that variants of the specific sequence as described above are included in these embodiments.

A sixth aspect of the invention relates to a method of genetically engineering the nucleic acid molecules of the second aspect encoding the chimeric opsin GPCR proteins of the first aspect of the invention.

In addition the sixth aspect of the invention relates to a method of engineering a nucleic acid molecule encoding a chimeric C-terminal peptide comprising a proximal region of an upstream opsin CT, in particular according to the first aspect of the invention and a target GPCR CT.

The conserved 3D structure common to all GPCR proteins and in particular also the ubiquitously conserved motifs in GPCRs, such as E(D)RY (SEQ ID NO: 80) around junction TM3/IL2, E around junction of IL3 with TM6, NPxxY (SEQ ID NO: 81) around the distal end of TM7 and NR(K)Q (SEQ ID NO: 82) around the proximal end of helix 8 or partially conserved elements such as palmitoylation sites at the distal end of H8 and further conserved elements between GPCRs are easily identified by sequence alignment with the prototype GPCR bovine rhodopsin.

Exemplary suitable splicing sites are also readily identified by optional structural alignment of transmembrane domain/intracellular domain junctions GPCR and by scanning the sequences in particular around junctions between the transmembrane and intracellular domains for conserved sequence motifs.

Preservation or reconstitution of conserved motifs or functional variants thereof of one or more selected parent GPCR (parent opsin/parent target GPCR) at chimeric junctions between them results with high probability in a functionally active chimeric GPCR.

Accordingly, in the sixth aspect of the invention, an efficient and simple method of genetically engineering and designing chimeric GPCRs with the desired functionalities such as light sensitivity of opsins, correct intracellular trafficking, efficient G-protein binding and G-protein specificity of target GPCRs is provided. The method requires only a single obligatory fusion site between the upstream opsin and the target CT. The desired truncation site of the upstream opsin is readily identified based on knowledge of a) the location of H8 or one or more palmitoylation sites—or “putative” palmitoylation site(s) corresponding to one of the palmitoylation sites in bovine rhodopsin as described above or by counting 7 to 13, in particular 8 to 12, more particularly 9 to 11 or approx. 10 amino acids downstream of the NR(K)Q (SEQ ID NO: 82) site and b) knowledge of the location of the C-terminus of the target GPCR.

The conserved 3D structure despite minimal sequence homology among GPCRs of different classes demonstrates an enormous tolerance of amino acid sequence variation in functional domains of GPCR proteins. Furthermore, engineering functional chimeric opsin GPCRs is simplified by splicing at or around conserved structural elements or motifs while preserving the conserved sequence or functional analogs thereof obtained e.g. by conservative amino acid substitutions. In some embodiments splicing sites are deliberately positioned at corresponding positions within conserved motifs of the parent GPCR and reconstituted in the same or a functionally equivalent version. This rational applies in particular to splicing at the NR(K)Q motif (SEQ ID NO: 82) or at a palmitoylation site positioned around the proximal and distal ends of the O-CT-proximal region, respectively.

Some embodiments of the method of engineering the nucleic acid molecule according to the second aspect of the invention, prior to the selection of a truncation site in the upstream opsin CT and/or a cutting site in the target GPCR CT, conserved motifs in one or both of the parent GPCRs and the genes encoding them are identified by including the steps of
aligning the amino acid sequence of the upstream opsin (or a fragment thereof) with the amino acid sequence of the target GPCR (or a fragment thereof) using a sequence alignment tool,
determination of amino acid positions constituting a conserved motif selected in particular from the group of conserved motifs comprising E(D)RY (SEQ ID NO: 80), E around the junction of IL3 with TM6, NPxxY (SEQ ID NO: 81) around the TM7/CT junction, NR(K)Q (SEQ ID NO: 82) and palmitoylated C in the CT and—provided the target GPCR is an opsin—K for binding of a chromophore in TM7.

In some of these embodiments, the amino acid sequence of the opsin is optionally aligned with the amino acid sequence of bovine rhodopsin for identification of amino acid positions constituting a conserved motif. Suitable alignment tools include e.g. Clustal Omega (EMBL-EBI) and other alignment tools as described above.

In some of these and other embodiments of the method of engineering the nucleic acid molecule encoding the chimeric opsin GPCR or the peptide comprise prior to the selection of a truncation site in the upstream opsin CT and/or a cutting site in the target GPCR CT an identification of conserved 3D GPCR domains or subdomains, in particular a subdomain helix 8, in one or both of the parent opsin and the parent target GPCR comprising the step of inputting the primary amino acid sequence into a program for prediction of secondary/tertiary protein structure. Suitable programs are available in the art such as e.g. YASPIN (Lin et al., 2005) or another program selected e.g. from the following list https://molbiol-tools.ca/Protein_tertiary_structure.htm or Kuhlmann et al., 2019) or the Schrodinger software package (https://www.schrodinger.com/prime). Methods available in the art as described above, in particular including recombinant nucleic acid technology, recombinant cloning design in silica and chemical nucleic acid synthesis, are known to the person skilled in the art.

Accordingly, in the sixth aspect of the invention a method of genetically engineering a nucleic acid molecule is provided, that encodes a chimeric opsin GPCR protein or a peptide, in particular as described above, that comprises a chimeric C-terminal domain (chimeric CT) comprising a truncated opsin CT comprising an O-CT-proximal region and that further comprises an essentially complete target GPCR CT or a functional derivative thereof. The chimeric CT is derived from a parent upstream opsin CT and from a parent target GPCR CT. The method of genetically engineering comprises the steps:
A-1 selecting a truncation site (x) in the CT of the parent upstream opsin at an amino acid position at the distal end of the O-CT-proximal region or within a distal extension to the O-CT-proximal region proximal region,
A-2 obtaining a nucleic acid molecule encoding an upstream opsin portion or a peptide with a truncated CT that is truncated at the selected truncation site;
B-1 selecting a cutting site (y) within a proximal region of the target GPCR CT, in particular at or upstream of an NR(K)Q motif (SEQ ID NO: 82) or between an NPxxY (SEQ ID NO: 81) and a NR(K)Q motif (SEQ ID NO: 82),
B-2 obtaining a nucleic acid molecule encoding a target GPCR CT or a functional variant in particular a functional fragment thereof; and
C-1 fusing the nucleic acid molecule encoding the truncated opsin-CT obtained in step A-2 with the nucleic acid molecule encoding the target CT or the functional variant thereof obtained in step B-2.

In some embodiments of the method of genetically engineering the nucleic acid molecule encoding the chimeric opsin GPCR or the peptide in step A-1 the truncation site (x) fulfills one of the following criteria:
the truncation site (x) is positioned at a nucleotide located at or at least 7 or 8 or 9 or 10 or 11 or 12 or 13 amino acids downstream of the NR(K)Q motif (SEQ ID NO: 82),
the truncation site (x) is positioned downstream of and in particular distally adjacent to the palmitoylation site or an amino acid corresponding to a palmitoylation site
the truncation site is positioned up to at most 45 or 47 or 49 nucleotides downstream of the NR(K)Q motif (SEQ ID NO: 82).

In some embodiments of the chimeric opsin GPCR, in particular in embodiments with an upstream opsin comprising an extensive C-terminus with an unusually large number of amino acids, such as melanopsin, the truncation site (x) is positioned at an amino acid position downstream of the distal end of the O-CT-proximal region in particular at a distal end of a distal extension to the O-CT-proximal region according to the description above. The distal end of the distal extension to the O-CT-proximal region is positioned in particular up to 30 or 31 or 32 or 33 or 34 or 35 amino acids downstream of the distal end of O-CT-proximal region or up to at most 45 or 47 or 49 nucleotides downstream of the NR(K)Q (SEQ ID NO: 82) motif, respectively.

In some embodiments comprising an upstream opsin with an extensive CT such as melandopsin, the truncation site (x) is positioned downstream of a cluster of conserved phosphorylation sites that contribute to response termination of photoactivation. Such conserved phosphorylation sites are in particular located between amino acid positions corresponding to positions 381 and 397 of mouse melanopsin as described by Mure et al. 2016. In other words, in these embodiments the distal end of the distal extension to the O-CT-proximal region is preferably selected downstream of or in particular distally adjacent to the distal end of said cluster of conserved phosphorylation sites.

In some embodiments of the method of genetically engineering the nucleic acid molecule encoding the chimeric opsin GPCR or the peptide, the truncation site x in the upstream opsin selected in step A-1 and the cutting site y of the target GPCR selected in step B-1 are both positioned at their respective palmitoylation sites or at an amino acid position corresponding to the palmitoylation site or are both positioned between 7 and 13, in particular between 8 and 12, more particularly between 9 and 11 or at 10 amino acids downstream of the NR(K)Q (SEQ ID NO: 82) site.

Some embodiments of the method of genetically engineering the nucleic acid molecule encoding the chimeric opsin GPCR or the peptide comprise one or more additional step for exchanging or partially exchanging one or more intracellular loop, e.g. for replacing at corresponding positions, one or more intracellular loop or partial intracellular loop of the upstream opsin by an intracellular loop or partial intracellular loop of the target GPCR.

wherein in particular one or more splicing site is selected from the group of splicing sits positioned at
a junction a and a junction b for exchange of IL1,
a junction c and a junction d for exchange of IL2,
a junction e and a junction f for exchange of IL3,
two splicing sites within IL3 that remove a highly variable region of the upstream opsin IL3 in exchange for IL3 of the target GPCR.

A seventh aspect of the invention relates to medical applications using the above described products related to the chimeric opsin GPCR. The medical applications include in particular medicaments and methods for treatment of a human or a non-human individual in need thereof. The products according to all previously described aspects and embodiments of the invention are applicable to the seventh aspect, i.e. for medical applications.

The products according to the above described aspects of the invention applicable for medical use, in particular for use in gene therapy, are selected from a group of products comprising
a chimeric opsin-GPCR protein according to the first aspect of the invention
a nucleic acid molecule encoding said opsin GPCR protein according to the second aspect of the invention
a capsid or a nucleic acid molecule encoding said capsid according to the third aspect of the invention
a vector according to the fourth aspect of the invention
a carrier or a cell according to the sixth aspect of the invention.

In some embodiments, the seventh aspect of the invention relates to medical treatment, in particular in the form of a gene therapy, of patients suffering from partial or complete loss of vision. In some of these embodiments the products comprise or encode a chimeric opsin GPCR comprising an opsin and mGluR6 or comprising two opsins.

In this text, embodiments comprising two opsins, i.e. comprising an upstream opsin and a target opsin, are also termed chimeric opsin-opsin (GPCRs) for short. Embodiments comprising an upstream opsin and mGluR6 are also termed opsin-mGluR6 for short.

Some preferred embodiments of the chimeric opsin-mGluR6 for medical treatment comprise melanopsin or any other opsin—such as e.g. box jelly fish opsin, parapinopsin or jumping spider rhodopsin or a humanized variant thereof or a cone opsin—as upstream fused to mGluR6 as the target GPCR. Some other preferred embodiments of the chimeric opsin GPCR for medical treatment comprise two opsins and include in particular any opsin fused to the target GPCR derived from a cone opsin or rhodopsin.

In some of these preferred and in further embodiments of the chimeric opsin-mGluR6 for medical treatment, the upstream opsin is truncated at a distal end of an O-CT-proximal region, in particular at a palmitoylation site as described above such as e.g. in the exemplary Mela(palm)-mGluR6 described herein; in other of these preferred and further embodiments the upstream opsin is truncated at a distal end of a distal extension to the O-CT-proximal region, in particular approx. 33 amino acids downstream of the palmitoylation site as described above such as e.g. in the exemplary Mela(palm+33AA)-mGluR6 described herein.

In some of preferred embodiments of the medical use according to the seventh aspect of the invention, in particular the gene therapy, to improve vision, to treat partial or complete loss of vision according to the seventh aspect of the invention, the transgene is operably linked to an ON-bipolar cell specific promoter, in particular to a 770En_454P(hGRM6) or to a 444En_454P(hGRM6) promoter.

In some of preferred embodiments of the medical use according to the seventh aspect of the invention to treat partial or complete loss of vision, a vector for gene therapy, in particular an rAAV vector, is applied. In some of these and other embodiments an AAV capsid, in particular AAV2(7m8), AAV2(BP2) or an AAV2 with a peptide insert as described above is applied.

In some embodiments of the seventh aspect of the invention, the chimeric opsin GPCR is for use in medical treatment of patients suffering from partial or complete loss of vision, a medical indication for the treatment is in particular selected from the group comprising retinitis pigmentosa (RP), age-related macular degeneration and any other form of photoreceptor degeneration.

The seventh aspect of the invention further relates to a pharmaceutical composition comprising a product according to the invention. In particular the pharmaceutical composition is provided in a suitable pharmaceutical formulation for administration into the eye.

In an exemplary embodiment, an AAV vector as described above is dissolved in a buffered saline solution for either sub-retinal or intra-vitreal injection into the eye. In some exemplary embodiments the AAV is dissolved in buffered saline (PBS) with 0.04% Tween-20 as gene therapeutic formulation.

Furthermore, the seventh aspect of the invention relates to a method of treating a human individual or a non-human individual, in particular an animal, in need thereof comprising the administration of a product selected from the group of products according to the invention. In some embodiments of the method, the chimeric opsin GPCR is administered by an intravitreal administration, in particular by an intravitreal injection, or by a sub-retinal administration.

In this text, the term intravitreal administration relates to a route of administration of a pharmaceutical agent, such as for example a nucleic acid molecule, a vector or a carrier for transfer, in which the agent is delivered into the vitreous body of the eye. Intravitreal administration is a procedure to place a medicament directly into the space in the back of the eye called the vitreous cavity, which is filled with a jelly-like fluid called the vitreous humour gel.

In this text, the term sub-retinal administration relates to a route of administration of a pharmaceutical agent, particularly a virus in the context of this specification, into the space between retinal pigment epithelium cells and photoreceptors.

The seventh aspect of the invention further relates to the use of the products according to the invention in the manufacture of a medicament for medical therapy to improve vision, or for the treatment of partial or complete blindness, or for the treatment of retinitis pigmentosa (RP), or for the treatment of macular degeneration or for the treatment of other forms of photoreceptor degeneration.

The seventh aspect of the invention also related to a medical application as described above comprising a product selected from the group of products comprising
a chimeric opsin-GPCR protein according to the first aspect of the invention
a nucleic acid molecule encoding said opsin GPCR protein according to the second aspect of the invention
a capsid or a nucleic acid molecule encoding said capsid according to the third aspect of the invention
a vector according to the fourth aspect of the invention
a carrier or a cell according to the sixth aspect of the invention,
   wherein the product comprises a chimeric opsin GPCR protein or comprises a nucleic acid molecule comprising a nucleic acid sequence encoding said chimeric opsin GPCR protein,
   wherein the chimeric opsin GPCR protein is selected from the group comprising
a Mela(palm)-mGluR6, in particular according to a sequence selected from the group comprising SEQ ID NO 18, SEQ ID NO 20, SEQ ID NO 22, SEQ ID NO 24, SEQ ID NO 26 and SEQ ID NO 28 or
a Mela(palm+33)-mGluR6, in particular according to SEQ ID NO 16 or
a Mela-mGluR6 additionally comprising an intracellular loop, in particular according to a sequence selected from SEQ ID NO 30 or SEQ ID NO 32.

Whenever an embodiment of a medical application according to the seventh aspect of the invention refers to a specific sequence according to a particular SEQ ID NO, it is understood that variants of the specific sequence as described above are included in these embodiments.

Below non-limiting, further exemplary details relating to some embodiments of the invention are presented e.g. in examples, tables, sequence listings, dependent claims, figure legends and figures. These exemplary embodiments are illustrative and not meant to limit the scope of the invention.

Wherever alternatives for single separable features are described as “embodiments”, it is to be understood that such alternatives may be combined freely and still remain within the scope of the invention is described herein.

Regarding some exemplary embodiments of the chimeric opsin GPCR protein with melanopsin as upstream opsin with a truncated CT, an exemplary truncation site is identified in the human and murine melanopsin amino acid sequences shown below. This truncation site is positioned at 33 amino acids downstream of the palmitoylation site (palmitoylated cysteine). This exemplary truncation site is termed “palm+33AA” site and it forms the distal end of the distal extension to the O-CT-proximal region.

In the sequence sections of the human and murine melanopsin gene (OPN4) shown below, the amino acid sequences of the C-terminal melanopsin fragment start at the proximal end of the O-CT-proximal region, i.e. at the NR(K)Q motif (SEQ ID NO: 82) (which in hOPN4 and mOPN4 is HPK). The palm+33AA truncation site is indicated by a downward arrow at amino acid position 397. The following amino acid residues are framed:
the conserved HPK, i.e. the NR(K)Q motif (SEQ ID NO: 82)
the palmitoylated cysteine
the conserved phosphorylated serine and threonine residues upstream of the truncation site.

hOPN4- SEQ ID NO 57

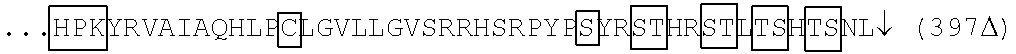

SWISIRRRQESLGSESEVGWTHMEAAAVWGAAQQANGRSLYGQGLEDLEAKAPPRPQ
GHEAETPGKTKGLIPSQDPRM
mOPN4- SEQ ID NO 58

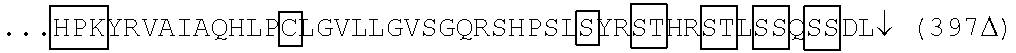

SWISGRKRQESLGSESEVGWTDTETTAAWGAAQQASGQSFCSQNLEDGELKASSSPQ
TKGHLPSLDLGM

In other exemplary embodiments of the chimeric opsin GPCR, in particular chimeric melanopsin GPCR, the truncation site of the truncated CT may be positioned at any amino acid position upstream of the “palm+33AA” site or further downstream, e.g. up to e.g. 34 or 35 amino acids downstream of the palmitoylation site.

Exemplary relevant conserved sites of parent opsins and parent target GPCRs that advantageously are conserved or reconstituted as functional derivatives in the chimeric opsin-GPCRs are provided below in the following Table I.

Exemplary tested splicing sites that yield functional chimeric opsin GPRC proteins are presented below in the following Table II.

In some embodiments splicing sites are placed at conserved motifs or sites in both parent GPCRs.

In some embodiments conserved motifs or sites serve as reference point for the identification of suitable splicing sites. Such splicing sites will be located at an equivalent distance to a particular conserved motif or site in both parent GPCRs such as the exemplary palm+33 site described above.

Generally, sequences of parent opsins are aligned based on conserved sites for the identification of suitable splicing sites to join domains or subdomains of two parent GPCRs (e.g. by cutting and subsequent ligation or by nucleic acid synthesis of the in silico designed chimeric opsin GPCR).

In some embodiments, where a parent opsin lacks known palmitoylation sites, both palmitoylatable sites or sites corresponding to palmitoylation sites in bovine rhodopsin were aligned to identify sites as suitable for splicing or as reference point.

TABLE 1

| | | Conserved Motif: | | | | | | |
|---|---|---|---|---|---|---|---|---|
| | | Function | | | | | | |
| | | Ionic Lock | | Structure | Chromophore Pocket | | Stabilization by | |
| | | Location | | | | | | |
| | | | Junction | | Lys in | | | |
| | | Junction | IL3 | Junction | TM7 | Negative | | Palm |
| | | TM3 with | with | TM7 | linked to | Counter- | Proximal | site |
| | | IL2 | TM6 | with CT | retinal | ion | end of H8 | (Cys) |
| | | Exemplary GPCR | | | | | | |
| | | Opsin | | | | | | |
| | | E (D) RY | | NPxxY | | | NR (K) Q | |
| | | (SEQ ID | | (SEQ ID | | | (SEQ ID | |
| Name | Gene | NO: 80) | E | NO: 81) | K | E | NO: 82) | C |
| bRhodopsin/ | RHO | E134-Y136 | E247 | N302-Y306 | K296 | E113 | N310-Q312 | C322 |
| | | | | | | | | C323 |
| hMelanopsin | OPN4 | D167-Y169 | E291 | N346-Y350 | K340 | E215 | H354-K356 | C367 |
| mMelanopsin | OPN4 | D166-Y168 | E288 | N343-Y347 | K337 | E214 | H351-K353 | C364 |
| hOPN1SW | hOPN1SW | E131-Y133 | E244 | N299-Y303 | K293 | E110 | N307-Y309 | C319 |
| hOPN1MW | hOPN1MW | E150-W152 | E263 | N318-Y322 | K312 | E129 | N326-Q328 | G338*, |
| | | | | | | | | K339* |
| hOPN1LW | hOPN1LW | E150-W152 | E263 | N318-Y322 | K312 | E129 | N326-Q328 | G338*, |
| | | | | | | | | K339* |
| Box jellyfish | JellyOP/ | D103-Y105 | E224 | N281-Y285 | K275 | E63*/A82 | H289-Q291 | C301 |
| opsin | cubOP | | | | | | | |
| hEncephalopsin | hOPN3 | E138-Y140 | E250 | N305-Y309 | K299 | D117 | I313-K315 | C325 |
| Neuropsin ? | hOPN5 | D130-Y132 | E247 | N302-Y306 | K296 | E177 | D310-K312 | A323$ |
| Parapin opsin | PPO | D120-F122 | E231 | N286-Y290 | K280 | E167 | N295-Q297 | C307 |
| Jumping spider | JSR1 | D147-Y149 | E272 | N327-Y331 | K321 | E194 | H335-K337 | C348 |
| rhodopsin 1 | | | | | | | | |
| | | Target GPCR | | | | | | |
| | | E (D) RY | | NPxxY | | | NR (K) Q | |
| | | (SEQ ID | | (SEQ ID | | | (SEQ ID | |
| Name | Gene | NO: 80) | E | NO: 81) | NA | NA | NO: 82) | C |
| Human and | hmGluR6 | N673-1675 | E781 | V837-Y841 | — | — | H846-E848 | — |
| mouse | GRM6 | | | | | | | |
| metabotropic | mmGluR6 | | E775 | V831-Y835 | — | — | H840-E842 | — |
| glutamate | | | | | | | | |
| Receptor 6 | | | | | | | | |
| Mouse | 5-HT7 | N391-D393 | E325 | N383-Y387 | — | — | | C404 |
| serotonin | HTR7 | | | | | | | |
| receptor 7 | | | | | | | | |

Notes:
NA = not applicable for non-opsin GPCRs according to invention
$ not palmitoylated, amino acid corresponding to C322/C323 of bovine rhodopsin

TABLE 2

| | | Amino Acid Sequence at Splicing Site | Parent | | | | |
|---|---|---|---|---|---|---|---|
| | | Opsin sequence: roman letters | Opsin | | Distal | | |
| | | Target GPCR sequence: italic letters | Protein | Proxi- | Domain/ | | |
| | | Conserved motifs and palm sites: | Name | mal | Region | | Con- |
| SEQ | | framed | abbrev./ | Domain | if | Parent | trib- |
| ID | Splicing | Helix 8 underlined, NA- not | Gene | or | appli- | Target | uted |
| NO | Sites | applicable | Name | Region | cable | GPCR | domain |
| 45 | palm | 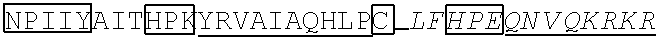 | Mela | trunc. | NA | mGluR6 | CT |
| | | 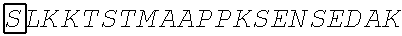 | hOPN4 | CT | | | |
| 46 | palm + | 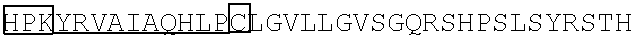 | Mela | trunc. | NA | mGluR6 | CT |
| | 33 | 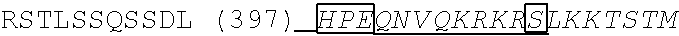 | hOPN4 | CT | | | |
| | | AAPPKSENSEDAK | | | | | |
| | | (397 indicates amino acid position distal | | | | | |
| | | to conserved phosphorylation sites) | | | | | |
| 47 | Jct. a | GMLGNLTVIYTF_MRHNDTPIVRASGREL_FIINLA | Mela | TM1 | TM2 | mGluR6 | IL1 |
| | Jct. b | VSDFLM | hOPN4 | | | | |
| 48 | Variable | IFIFRA↓IRETG_RGVPETFNEA_PLRQRRQWQRLQ | Mela | proxim | Distal | mGluR6 | IL3 |
| | region | SEWKMAK↓VALIVIL | hOPN4 | IL3 | IL3 | | |
| | | (arrows indicate splice sites at the membrane/ | | region | region | | |
| | | cytosol interface used in the original | | | | | |
| | | Chimeric opsin mGluR6 proteins) | | | | | |
| 49 | palm | 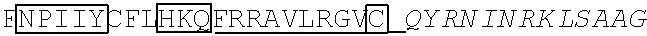 | Jellyops | trunc. | NA | 5HT7 | CT |
| | | MHEALKLAERPERSEFVLQNCDHCGKKGHDT | in | CT | | | |
| | | | cubop | | | | |
| 50 | palm | 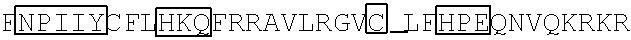 | JellyOP | trunc. | NA | mGluR6 | CT |
| | | 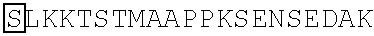 | cubop | CT | | | |
| 51 | palm | 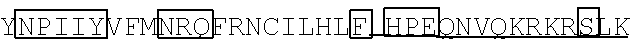 | Cone | trunc. | NA | mGluR6 | CT |
| | | KTSTMAAPPKSENSEDAK | opsin | CT | | | |
| | | | hOPN1MW | | | | |
| 52 | Palm | 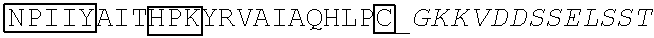 | Mela | Trunc. | NA | Cone | CT |
| | | SKTEVSSVSSVSPA | hOPN4 | CT | | opsin | |
| | | | | | | OPN1MW | |
| 54 | Jct.a | TGMLGNLTVIY_ATMKFKKLRH_PANMFIINLAVSD | Mela | TM1 | TM2 | Cone | IL1 |
| | Jct.b | FL | hOPN4 | | | opsin | |
| | | | | | | OPN1MW | |
| 55 | Jct.c | 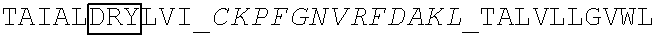 | Mela | TM3 | TM4 | Cone | IL2 |
| | Jct.d | YALAWSLPPF | hOPN4 | | | opsin | |
| | | | | | | OPN1MW | |
| 56 | Jct.e | LIIIFCYI_QVWLAIRAVAKQQKESESTQKAEKEVT | Mela | TM5 | TM6 | Cone | IL3 |
| | Jct.f | _KVALIVILLFVL | hOPN4 | | | opsin | |
| | | | | | | OPN1MW | |

Notes regarding abbreviations of splicing sites:
Palm = Palmitoylation site or amino acid position corresponding to palmitoylation site
Palm + 33 = Amino acid position 33 amino acids downstream of Palm
Jct. = Junction at transition between a transmembrane domain and an intracellular domain
Variable region = two splice sites excising a highly variable region of an IL of an opsin that is replaced by an IL of a target GPCR
Exemplary splice site x-3 for embodiments with two opsins

### EXAMPLES

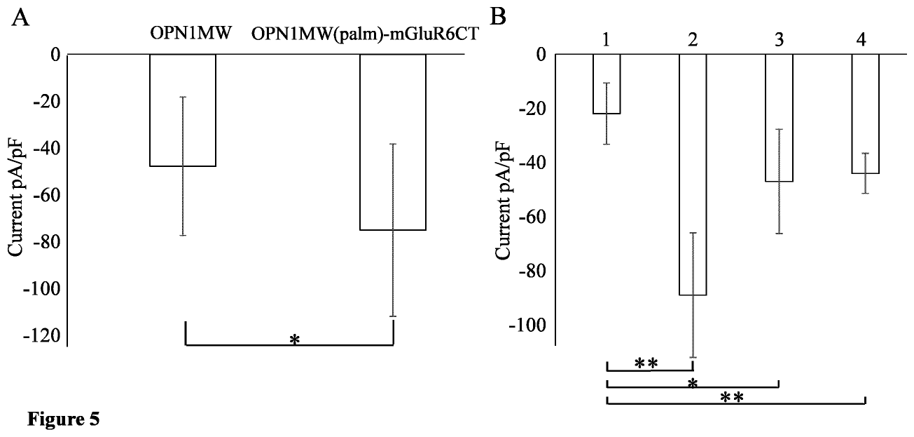

FIGS. 5 to 12 show besides microscopic visualizations results from three different experimental approaches applied to prove the function of the chimeric opsin GPCR proteins:

### In-Vitro:

1. HEK-GIRK patch-clamp Gβγ activity assay: Functional opsins were expressed in a HEK293 cell line stably expressing GIRK (Kir3.1/3.2) potassium channels. A light stimulus activates the opsin, which activates endogenous intracellular Gi/o-proteins. The activated Gβγ proteins in turn open GIRK ion channels resulting in an electrical response that is time-locked to the light stimulus and can be recorded. For a detailed description of the method refer to (van Wyk et al., PLoS Biol 2015)
2. Gα-specificity bioluminescence plate reader assay: Each opsin was co-expressed with reporter-expressing plasmids in a HEK293 cell line. Similar to the GIRK assay, activated opsins activate G-proteins that in turn inhibit or activate enzymes that generate cAMP (Gs and Gi) or Ca2+(Gq). The accumulation of these products (cAMP and Ca2+) can be measured by the light emittance of a bioluminescent protein activated by cAMP or Ca2+, respectively. To visualize changes in Gs and Gi activity, pcDNA5/FRT/TO Glo22F was used as reporter plasmid, whilst for changes in Gq signaling, pcDNA5/FRT/TO mtAeq was used as reporter plasmid. Luciferase or Coelenterazine, respectively, were added as substrate and changes in cAMP (Gs and Gi) or Ca2+(Gq) levels indicated by changes in luminescence measured with an Infinite F200Pro Tecan plate reader (Mannedorf, Switzerland). To normalize the light-induced fluorescence changes to the opsin transfection levels, the absolute changes in fluorescence were divided by the overall mCitrine re-porter fluorescence of the respective well of the measured plate.

### Ex-Vivo:

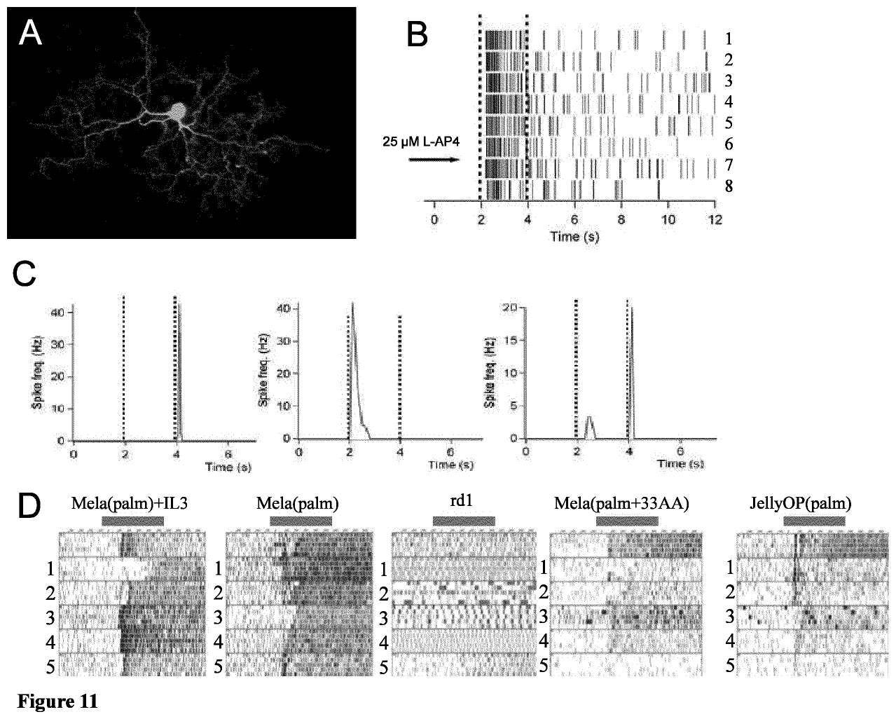

We have recorded light responses from mouse retinal neurons in retinas without photoreceptor cells (rd1 retinitis pigmentosa mouse, C3H/HeOuJ mouse line) where these opsin proteins were introduced to the surviving retina by an AAV gene therapy (cf. van Wyk et al. (2015) and legend of FIG. 11 below).

### In-Vivo:

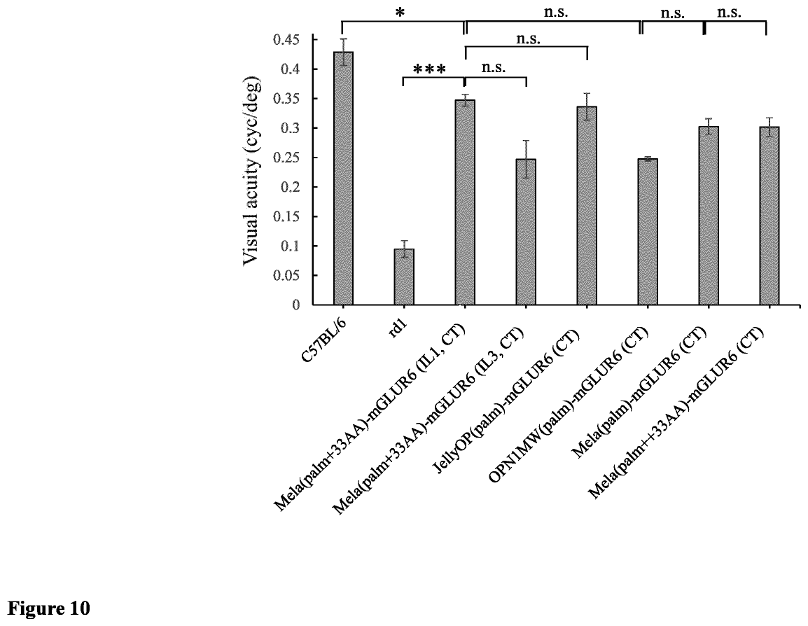

We recorded behavioral optokinetic reflexes from mouse models with photoreceptor degeneration where these opsin proteins were introduced to the surviving retina by an AAV gene therapy (cf. legend to FIG. 10 below).

### DETAILED DESCRIPTION OF THE FIGURES

The invention will be better understood and objects other than those set forth above will become apparent when consideration is given to the following detailed description thereof. Such description makes reference to the annexed drawings, wherein:

FIG. 1: General structure of an opsin.

FIG. 1 shows a schematic drawing of a general structure of a parent opsin with seven transmembrane domains TM1 to TM7, extracellular domains, N-terminus NT and extracellular loops EL1, EL2 and EL3 as well as intracellular domains C-terminus CT and intracellular loops IL1, IL2 and IL3. Junctions between the TM domains and intracellular domains at a border between membrane and cytoplasm are indicated as junctions (a) to (g). Optional splicing sites for an opsin-GCPR chimeric protein may be located e.g. at these junctions for exchange of intracellular loops. Furthermore, a conserved subdomain of the CT, helix 8 (H8) in a proximal region of the CT is indicated as well as are several conserved sequence motifs present in opsins, in particular:
an ionic lock between a E(D)RY (SEQ ID NO: 80) site at the cytoplasmic border of TM3 linked to a glutamate residue (E) at the junction (f) between IL3 and TM6,
a chromophore binding pocket with a lysine residue (K) in TM7 bound to a chromophore 11-cis-retinal via a Schiff base and a negative counterion, typically a glutamate, in TM3 stabilizing the Schiff base,
a NPxxY motif (SEQ ID NO: 81) at the C-terminal end of TM7,
one or more palmitoylation site(s) (C) at a distal end of H8,
C-terminal phosphorylation sites (P) in a cytoplasmic region of the CT,

In addition, FIG. 1 indicates three exemplary truncation sites in the CT, (x-1), (x-2) and (x-3), as examples.

The depicted truncation site (x-1) is located at a distal end of H8 and distally adjacent to a palmitoylated cysteine residue corresponding to the palmitoylation site in bovine rhodopsin (C322 or C323). The depicted truncation site (x-2) is located downstream of the palmitoylation site, for melanopsin 33 amino acid downstream of the palmitoylation site or up to 40, 41, 42, 43, 44 or 45 amino acids downstream of the NR(K)Q motif (SEQ ID NO: 82). The depicted truncation site (x-3) lies within or directly distal to the NR(K)Q motif (SEQ ID NO: 82). Other not depicted truncation sites are in particular located at amino acid positions between (x-1) and (x-2) or between (x-3) and (x-1).

FIG. 2: Scheme of an exemplary chimeric opsin GPCR.

FIG. 2 shows an exemplary embodiment of the chimeric opsin GPCR requiring only minimal genetic engineering with only a single splicing site (x-1) where the truncated C-terminus of an exemplary parent upstream opsin is cut distally adjacent to the palmitoylation site and is fused with the CT of an exemplary target GPCR; alternative exemplary splice sites such as (x-2) and (x-3) are indicated and described in FIG. 1

The exemplary embodiment shown in FIG. 2 further comprises additional sequences that may be optionally added at the very distal end of the C-terminus. Such optional additional sequences may encode marker proteins (e.g. fluorescent proteins) or trafficking sequences (e.g. Golgi and ER export signals or membrane trafficking sequences). In further exemplary embodiments not shown here optional additional splicing sites can be introduced around junctions (A′)-(G′) between the TM domains (TM1 to TM7) and the extracellular domains (NT, EL1 to EL3) for extracellular domain exchange with human opsin domains to reduce antigenicity of the protein in a potential human therapy if nun-human opsins re used.

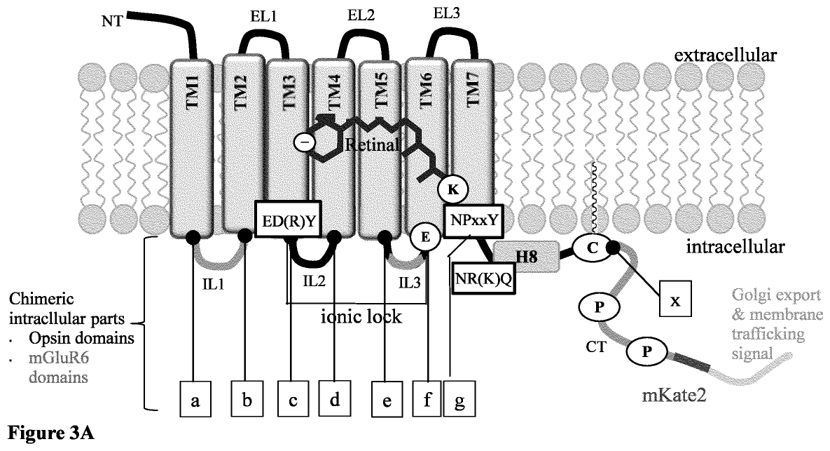

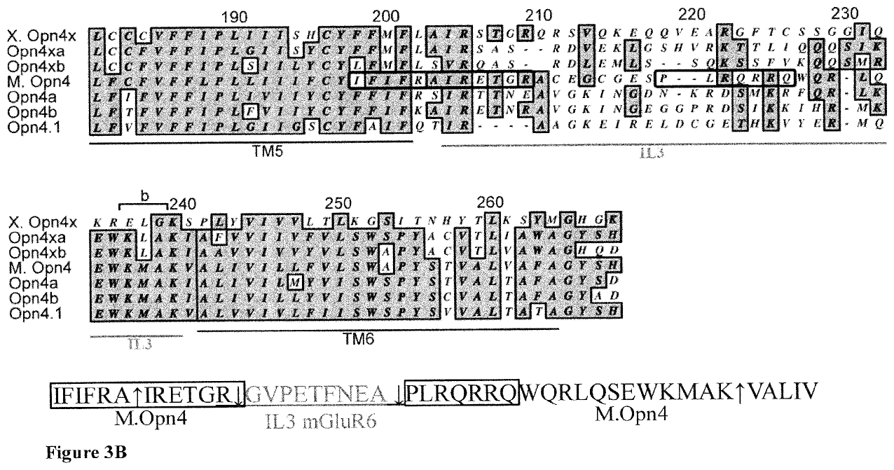

FIG. 3: Exemplary embodiment of a chimeric opsin mGluR6.

(A): FIG. 3 shows an exemplary embodiment of a melanopsin-mGluR6 chimeric GPCR with a chimeric C-terminus, containing the truncated melanopsin C-terminus up to and including the palmitoylation site (C) followed distally adjacent to the palmitoylation site in distal direction first by the full-size mGluR6 CT, then next followed by the mKate2 fluorescent marker and finally by an additional Golgi export signal and a rhodopsin membrane trafficking sequence located at the very distal end of the target CT. Further, IL1 of melanopsin has been replaced in full by that of mGluR6 at cutting splicing sites positioned at the junctions a and b, whereas the full IL3 of mGluR6 has been introduced at splicing sites positioned within a highly variable region of the longer IL3 of melanopsin.

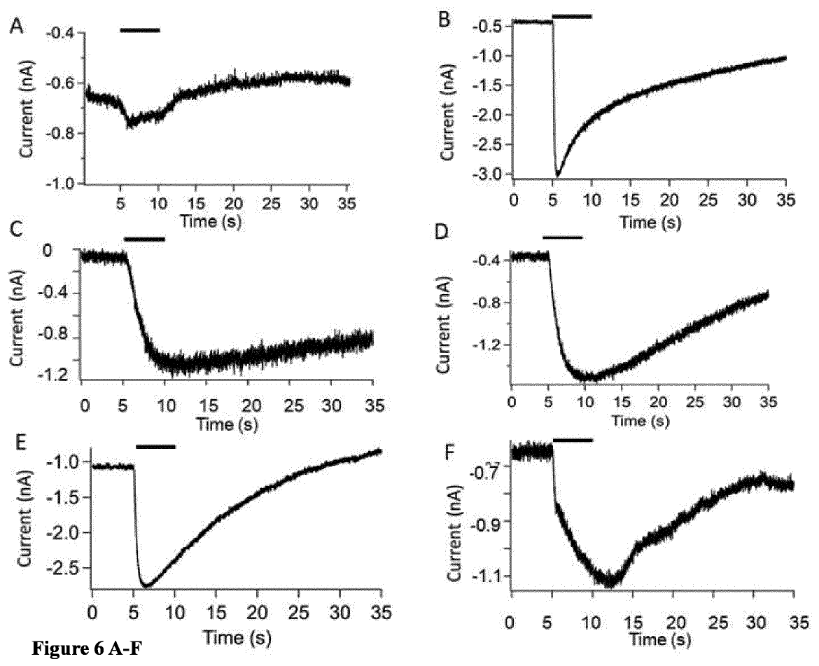

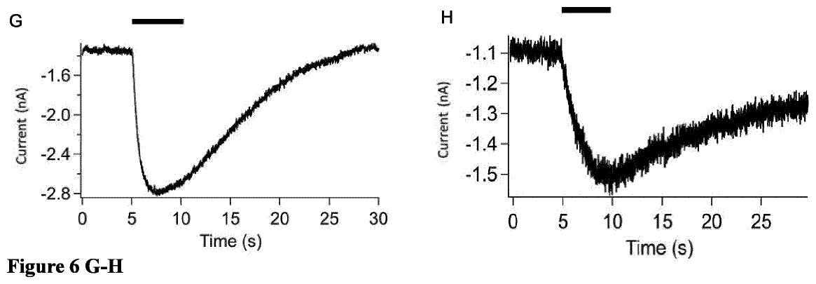

In this exemplary embodiment the short IL3 of mGluR6 is introduced into the least conserved region of the longer IL3 of melanopsin under the rationale that variable regions determine the functional differences, i.e. here potentially G-protein specificity. Indeed this embodiment with a chimeric opsin mGluR6 IL3 enhances the functionality compared to mela(palm)mGluR6 introduced into the opsin IL3 as demonstrated in FIG. 6B.

(B)-top: Alignment of melanopsin genes (OPN4) from different species (from DOI: 10.1371/journal.pone.0025111) indicating the hypervariable regions in IL3 between TM5 and TM6.

(B)-bottom: The insertion location of IL3 of mGluR6. Arrows pointing down indicate the cutting sites in mouse melanopsin (M. OPN4) utilized here, arrows pointing up indicate the junctions e and f indicated in A.

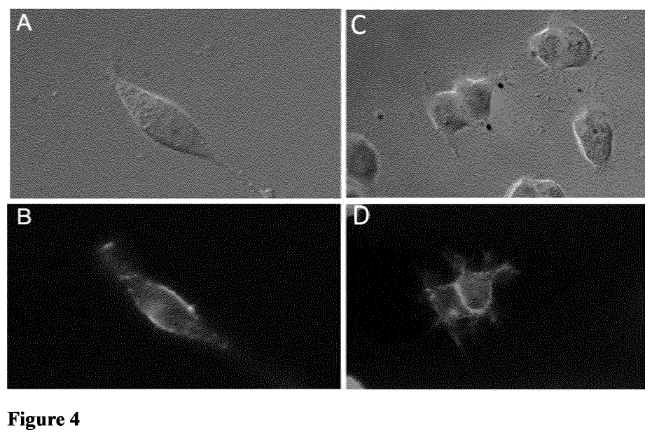

FIG. 4: Exemplary embodiments of chimeric opsin GPCRs target to the cell membrane.

To confirm proper intracellular trafficking to the plasma membrane, opsin-mGluR6-mKate2 fusion proteins were generated wherein the fluorescent reporter protein (mKate2) was used to study protein localization. The fusion proteins were expressed in HEK293 cells. By comparison of differential interference contrast microscopy images (A,C) with fluorescent images (B,D), it was verified that the Opsin-mGluR6 proteins were located in the cell membrane. This is shown with two different chimeric opsin GPCR proteins, namely a melanopsin-mGluR6-mKate2 chimeric GPCR in (A,B) and with a Jellyfish Opsin-mGluR6-mKate2 chimeric GPCR in (C,D). In both chimeric GPCRs the truncation site is positioned distally adjacent to the palmitoylation site of the melanopsin CT.

FIG. 5: Exemplary embodiments of chimeric opsin mGluR6 with a chimeric C-terminus showing increased light-activated currents mediated by opsin-mGluR6s as compared to the parent opsin.

Various exemplary chimeric opsin-mGluR6 constructs (lacking additional trafficking sequences) were transiently transfected into a HEK293 cell line stably expressing GIRK1/2 channels (potassium channels that are directly opened by activated G-proteins of the Gi/o family). When cells are patch-clamped in voltage-clamp mode (−75 mV holding potential), a 470 nm light stimulus (1×10¹⁴ photons cm⁻² sec⁻¹) presented for 5s activates an inward GIRK current depicted for the variants in the histograms. The relative sizes of GIRK currents activated by different constructs (normalised to the size of the patched cell in pF) are indicated. Stars indicate significance levels determined by a Student's t-Test (*p≤0.05, **p≤0.01)

(A) Middle-wave cone opsin (OPN1MW) induced GIRK currents are significantly smaller than those induced by chimeric OPN1MW(palm)-mGluR6CT.

(B) Comparative light-activated GIRK currents by melanopsin variants, 1=unmodified melanopsin, 2=melanopsin cut at the palmitoylation site with added CT mGluR6, 3=in addition the full IL1 of melanopsin has been replaced with that of mGluR6, 4=in addition the full mGluR6 IL3 has been placed into the variable position of the long IL3 of melanopsin. The C-terminal addition of the mGluR6 C-terminus significantly increases the light-induced GIRK currents.

FIG. 6: Example of in-vitro functional screening of chimeric opsin GPCRs using HEK-GIRK cells.

Transfected HEK-GIRK cells with opsin-target GPCR chimeras were assessed for their ability to activate the Gi/o G-protein signaling as described in Van Wyk et al. (2015). Shown are example light responses in patch-clamp traces recorded from HEK-GIRK cells transfected with various Opsin-GPCR variants carrying in addition to the target GPCR CT also an mKate fluorescent protein and additional Golgi and membrane trafficking sequences (light stimulus presented as black horizontal lines; 470 nm; 1×10¹⁴ photons/sec/cm²). (A) Prototype Opto-mGluR6 (WO 2012/174674 A1 and van Wyk et al. (2015)), (B) Mela(palm+33AA)-mGluR6, (C) Mela(palm)+IL1-mGluR6, (D) Mela(palm)+IL3-mGluR6, (E) Mela(palm)-mGluR6, (F) JellyOP(palm)-mGluR6, (G) OPN1MW(palm)-mGluR6, (H) Mela(palm)-OPN1MW(IL1, IL2, IL3, CT).

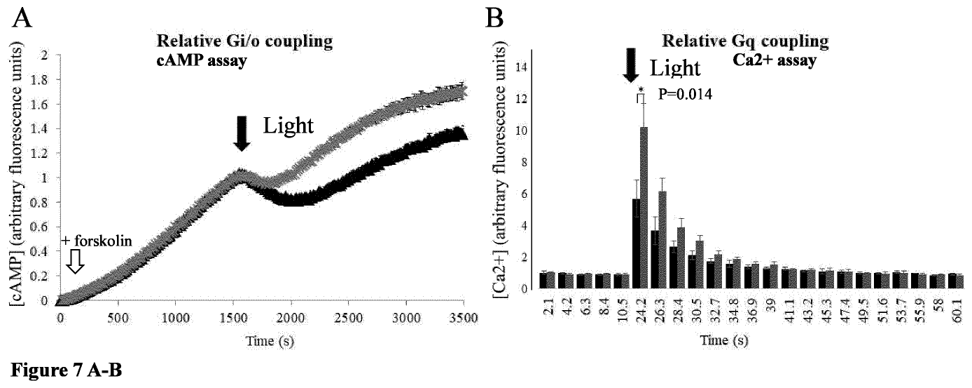

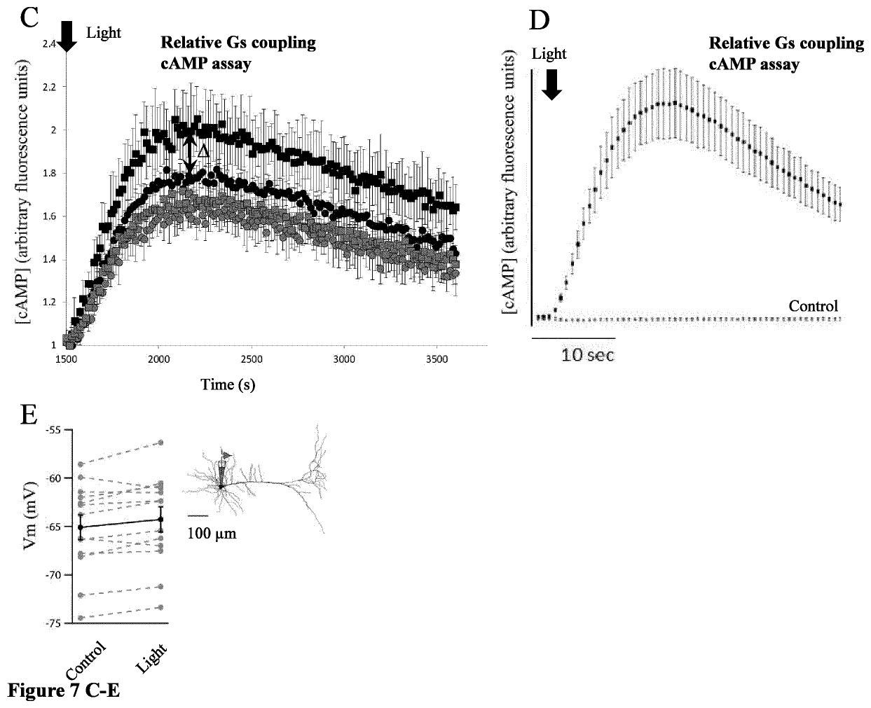

FIG. 7: Plate reader experiments probing for G-protein re-targeting and pathway selectivity of exemplary embodiments of chimeric opsin GPCRs.

We co-transfected HEK293 cells with Opsin-GPCR chimeras reporter-expressing plasmids and assessed their Gα-specificity in a bioluminescence plate reader assay. Similar to the GIRK assay, activated opsins activate G-proteins that in turn inhibit or activate enzymes that generate cAMP (Gs), reduce cAMP (Gi/o) or increase intracellular Ca2+(Gq). The accumulation of these products (cAMP and Ca2+) can be measured by the light emittance of a bioluminescent protein activated by cAMP or Ca2+, respectively. Light application (480 nm, 10 seconds) is indicated by the black arrows. To normalize the light-induced fluorescence changes to the opsin transfection levels, the absolute changes in fluorescence were divided by the overall mCitrine reporter fluorescence of the respective well of the measured plate. Black arrows indicate light stimulation. (A,B) Preference for Gi/o (A) and Gq (B) coupling of Mela(palm)-mGluR6 (black traces) and unmodified melanopsin (grey traces). Note that in (A), intracellular cAMP was first enhanced by addition of forskolin (stimulating adenylate cyclase) so that cAMP reduction by the light-activated chimeric protein can be measured. The graphs show, that the exchange of the C-terminus of melanopsin by that of mGluR6 at the palmitoylation site shifts the G-protein preference from Gq (melanopsin) to Gi/o (mGluR6). Inserting the C-terminus of mGluR6 at the palmitoylation site of melanopsin shifts the G-alpha subunit preference from Gq to Gi/o. (C) Gi/o vs. Gs coupling preference of JellyOP unmodified (grey traces) compared to JellyOP(palm)-mGluR6 (black traces). PTX is an inhibitor of Gi/o, indicating that JellyOP binds to Gs only (no change in signal without (•) and after addition of PTX (▪)), whereas JellyOP(palm)-mGluR6 clearly binds to Gi/o, visible as the differential luminance values (A) before addition of PTX (•) and clearly increased after addition of PTX (▪). Inserting the C-terminus of mGluR6 at the palmitoylation site of jellyfish opsin shifts the G-alpha subunit preference from Gs to Gi/o.

(D) JellyOP(palm)-5HT7 activates Gs efficiently indicated by light-activated cAMP increase. Control: only mCitrine expressing HEK293 cells, without light-activated chimeric protein. (E) JellyOP(palm)-5HT7 expressed in pyramidal cells of the anterior cingulate cortex reduce activity of HCN channels and thereby depolarize the membrane potential. This effect is identical to the effect of pharmacological 5-HT7 stimulation (Santello et al. (2015)). Data (bottom) from somatic patch-clamp recordings (shown on top) of pyramidal cells from acute slices of murine anterior cingulate cortex. JellyOP(palm)-5HT7 has been introduced by an AAVdj gene therapy by stereotactic injection.

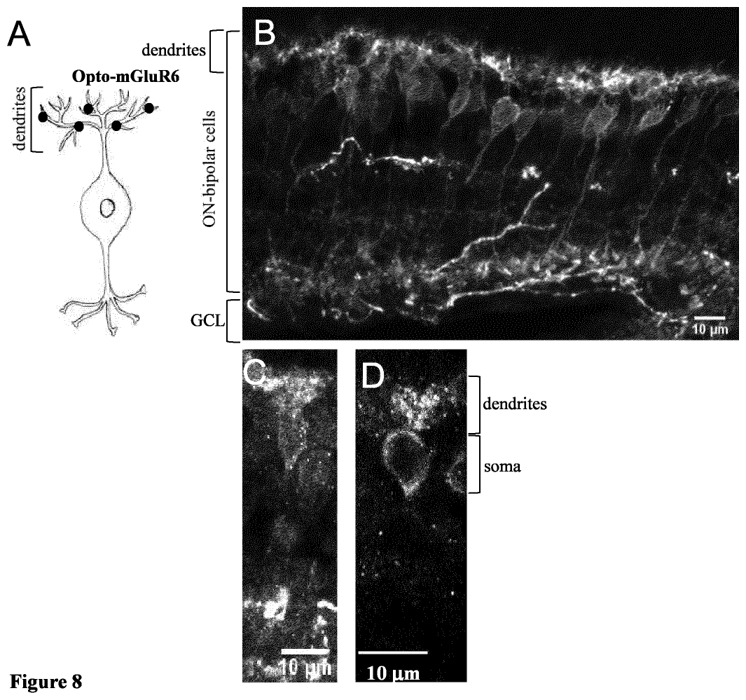

FIG. 8: Correct in vivo trafficking into the ON-bipolar cell dendrites and the mGluR6 signalosome of exemplary embodiments of chimeric opsin-mGluR6 variants.

Mice were treated with a gene therapy using ssAAV2(7m8) (Dalkara et al. (2013)) and setting the melanopsin-mGluR6 gene under control of the 770En_454P(hGRM6) promoter (EP19200082.6, attached to the filing of this application). (A) Sketch indicating the correct subcellular localization of chimeric opsin mGluR6 proteins in the dendrites of ON-bipolar cells, where also native mGluR6 resides. (B) Mela(palm+33AA)-mGluR6-IRES-TurboFP635 visualized with an anti-melanopsin antibody (white) is clearly expressed in the dendrites of ON-bipolar cells. The axons originating in the Ganglion cell layer (GCL) are from ipRGCs (intrinsically photosensitive retinal ganglion cells), naturally expressing melanopsin. Mela(palm)-mGluR6-mKate2 (C) and JellyOP(palm)-mGluR6-mKate2 (D) visualized with an anti-RFP antibody again clearly indicate the dendritic localization of the proteins. Opsin(palm) versions are sufficient to localize the chimeric protein correctly in the target cell.

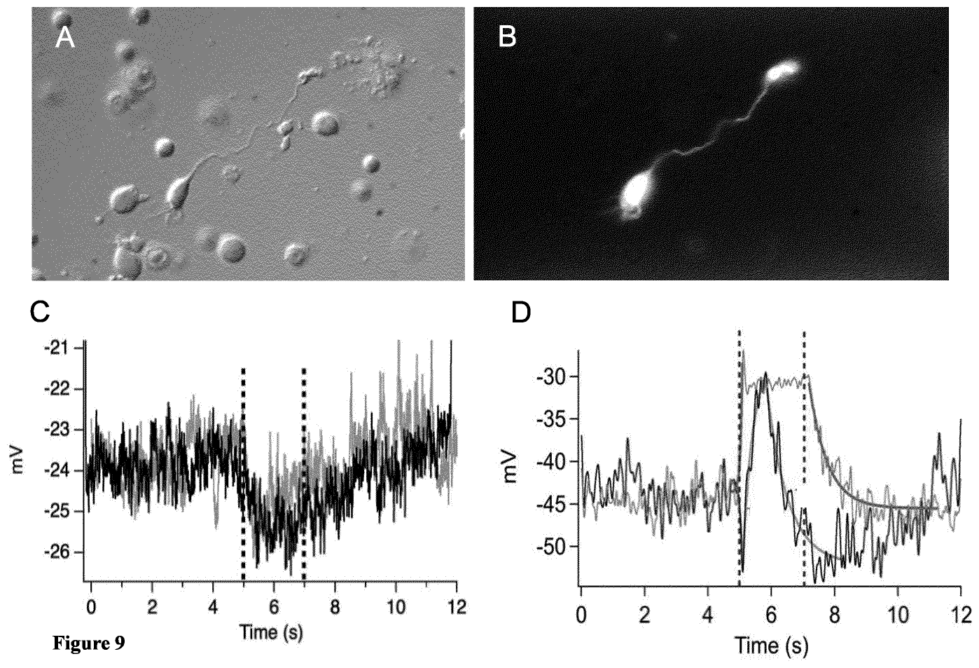

FIG. 9: Exemplary embodiments of chimeric opsin-mGluR6 GPCRs render isolated ON-bipolar cells directly light sensitive.

Mice were gene-therapeutically treated with Mela(palm+33AA)-mGluR6 and JellyOP(palm)-mGluR6 and retinas after enucleation digested with papain. Isolated cells were plated on glass cover slips and patch-clamped using the perforated patch technique. (A) Bipolar cells were easily identified under DIC optics. (B) Transfected bipolar cells were identified by co-expression of a fluorescent reporter gene, here TurboFP635 visualized under the fluorescent microscope. (C, D) Example patch-clamp recordings from transduced ON-bipolar cells in response to 2 seconds of blue light (470 nm; 1×10\hat{ }14 photons per cm\hat{ }per sec), indicated by the broken vertical lines. (C) Two overlaid example traces in grey and black from ON-bipolar cells expressing Mela(palm+33AA)-mGluR6. In response to light, the cells clearly hyperpolarize, indicative of direct activation of the mGluR6 cascade negatively gating the TRPM1 non-selective cation channel. (D) Comparative patch-clamp traces from an ON-bipolar cell expressing JellyOP(palm)-mGluR6 (black trace) and a rod bipolar cell directly activated by photoreceptors (grey trace) recorded in a retinal slice. JellyOP(palm)-mGluR6 expressing bipolar 3 cells showed extremely fast kinetics, with a response offset (Tau(off)) of 670 ms. This is virtually identical to the response offset of a bipolar cell under photoreceptor activation (Tau(off)=570 ms in this example). Also the response onset of JellyOP(palm)-mGluR6 (Tau(on)=90 ms) is virtually identical to the response onset in a bipolar cell activated by photoreceptors (70 ms). Endogenous, rapid kinetics is a clear indication for correct localization of JellyOP(palm)-mGluR6 in the mGluR6 signalosome and proper signal transmission within the bipolar cell. Fits of kinetic parameters (Tau values) are indicated by the red and green lines.

FIG. 10: In vivo measurements of visual acuities of blind mice that were treated by an AAV gene therapy with exemplary embodiments of chimeric opsin mGluR6 variants.

The histograms indicates average visual acuities (±s.e.m.) of blind Retinitis pigmentosa rd1 (retinal degeneration C3H/HeOuJ line) mice treated by an AAV gene therapy with different chimeric opsin-mGluR6 constructs indicated on the x-axis, wherein (palm) refers to the truncation site positioned distally adjacent to the palmytolation site in the CT of melanopsin, jellyfish opsin and middle wave cone opsin, Mela(palm+33AA) refers to the truncation site positioned distally adjacent to amino acid position 33 downstream of the palmytolation site in the melanopsin CT and wherein +IL1 or +IL3 refer to the presence of these subunits of mGluR6 in addition to the CT of mGluR6 and finally wherein JellyOP und OPN1MW refer to jellyfish opsin and human middle wave cone opsin, respectively. C57BL/6 refers to non-injected, seeing wildtype mice and is used as positive control. In this test, the mouse is placed unconstrained on an elevated platform surrounded by a virtual reality (Striatatech, optokinetic drum) showing black and white bars of changing spatial frequency (for details see Prusky et al. (2004)). The tracking head movements (optomotor reflex) of the mice are automatically monitored by an infrared camera and analysed in order to quantify the highest spatial acuity (cyc/deg) still perceived by the mice. Mela(palm)-mGluR6 injected mice perform significantly better than their blind littermates (rd1). All melanopsin-mGluR6 variant treated mice performed equally well, also the JellyOP(palm)-mGluR6 and OPN1MW(palm)-mGluR6 injected mice. Significance levels were determined by a one-way ANOVA test and in the graph the significance levels are indicated as: *p≤0.05, ***p≤0.001 and n.s. not significantly different. In summary, all constructs performed equally well in significantly restoring spatial vision in blind rd1 mice. The gene therapy was performed with ssAAV2(7m8) (Dalkara et al (2013) vectors setting the chimeric opsin mGluR6 proteins gene under control of the 770En_454P(hGRM6) promoter.

FIG. 11: Ex-Vivo Light responses recorded from retinal ganglion cells in blind rd1 retinas treated with novel Opsin-mGluR6 constructs.

Cell-attached patch-clamp recordings were performed ex vivo in whole-mount retinas. (A) Retinal ganglion cell labeled intracellularly after patch-clamp recordings for identification of cell type. (B) An exemplary raster plot showing the spike responses of a retinal ganglion cell in a rd1 retina treated with the Mela(palm+33AA)-mGluR6 chimera. Responses are not blocked by the mGluR6 receptor agonist, L-AP4 (25 μM), blocking the input from photoreceptors to ON-bipolar cells. This confirms that light responses are driven by Mela(palm+33AA)-mGluR6. Light was applied for 2 seconds between the stippled lines. Each horizontal line of bars (numbered 1-8) indicates one recording. Each vertical line corresponds to a recorded action potential of the ganglion cell. Clearly, this cell increases action potential firing when light is applied very reliably. (C) Example spike-time-histograms of transient OFF, ON and ON-OFF ganglion cells restored by Mela(palm)-mGluR6 expression in otherwise blind murine rd1 retina. The restoration of the natural diversity of ganglion cells responses to light—i.e. increase in spike frequency at light offset (left) termed OFF-cell, increase in spike frequency at light onset (middle), termed ON-cell or increase of firing at light on- and off-set (right), termed ON-OFF cell—confirms restoration of endogenous inner retinal function. (D) Multi-electrode array (MEA) recordings from rd1 retinal flat mounts of mice transduced with different chimeric opsin mGluR6 proteins variants. Shown are example raster plots (similar to B) from selected electrodes (numbered 1-5) of repeated light stimulation (duration of light stimulation indicated by horizontal bars above the traces). rd1 are untreated litter mates with no changes in basal firing rate upon light stimulation. In contrary, all retinas of chimeric opsin mGluR6 proteins show prominent light-locked responses.

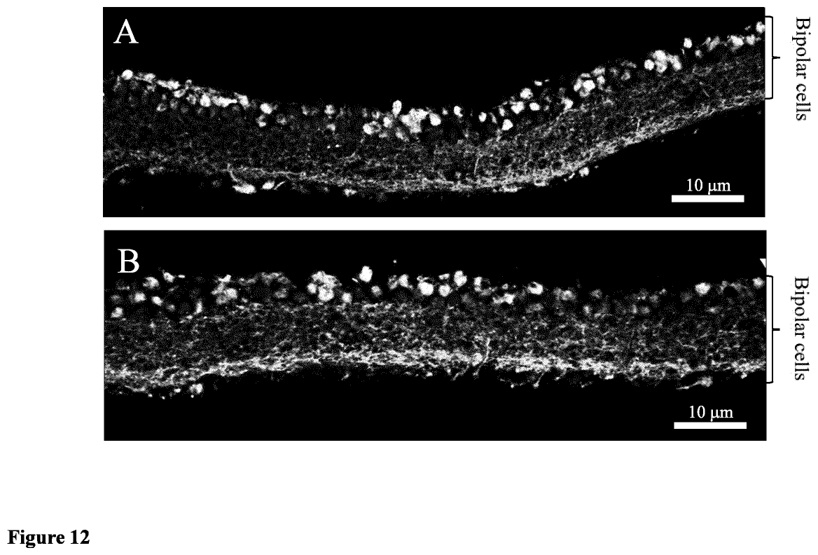

FIG. 12: Micrograph of vertical cryosections through the retinas from two treated degenerated mice (rd1 retinal degeneration mouse line C3H/HeOuJ) showing hMela(palm)-mGluR6-IRES2-TurboFP635 expressing ON-bipolar cells after an intravitreal gene therapy with AAV2 comprising the peptide inserts (a) NLAPRTPHTAAR (SEQ ID NO: 68) and (b) NLANHAPNHCAR (SEQ ID NO: 67) between N587 and R588 of the viral VP1 gene encoding the AAV2 capsid. Expression of hMela(palm)-mGluR6-IRES2-TurboFP635 is in both cases driven by the 770En 454P(hGRM6) ON-bipolar cell specific promoter.

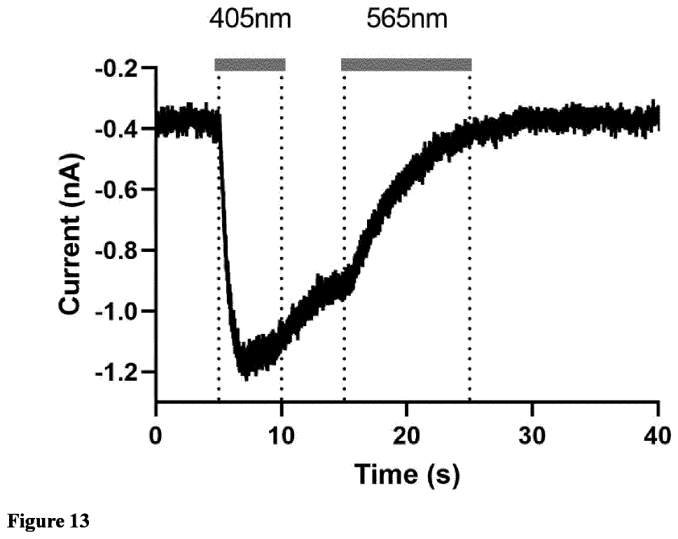

FIG. 13: An exemplary JSR1(S186F)palm-beta2AR chimeric opsin GPCR was expressed in HEK293-GIRK cells and light-induced currents were measured with the whole-cell patch-clamp method. 385 nm illumination induced GIRK currents, whereas 550 nm light terminated activity due to the bichromic nature of the bistable JSR1(S186F) mutant. An analoguous patch clamp experiment using the same illumination performed with an exemplary hJSR(S186F)palm-GABAB2 chimeric opsin GPCR induced analogous GIRK currents (data not shown).

cDNA and amino acid sequences for a selection of exemplary embodiments of the chimeric opsin GPCRS as presented in the overview of Table 3 below.

TABLE 3
Overview of exemplary embodiments with indication of parent GPCRs and C-terminal
splicing site and SEQ ID NO. Table 3 discloses “NR(K)Q” as SEQ ID NO:
82, “NRQFRN” as SEQ ID NO: 87, “CILHLF” as SEQ ID NO: 88, and “IAFQ” as SEQ ID NO: 90.

| | SEQ ID | | | | |
|---|---|---|---|---|---|
| | NO ¹⁾ | | | Parent | Position of |
| | and | | | target | splicing site in |
| Embodiment | origin²⁾ | Name ^{3), 4)} | Parent opsin | GPCR | CT target GPCR |
| A | 1; 2; m | Mela(palm + 33AA)-mGluR6 | melanopsin | mGluR6 | NR(K)Q = HPE |
| B | 3; 4; m | Mela(palm)-mGluR6 | melanopsin | mGluR6 | NR(K)Q = HPE |
| C | 5; 6; m | Mela(palm + 33AA) - | melanopsin | mGluR6 | NR(K)Q = HPE |
| | | mGluR6(IL1, CT) | | | |
| D | 7; 8; m | Mela(palm)-mGluR6(IL1, CT) | melanopsin | mGluR6 | NR(K)Q = HPE |
| E | 9; 10; m | Mela(palm + 33AA) - | melanopsin | mGluR6 | NR(K)Q = HPE |
| | | mGluR6(IL3, CT) | | | |
| F | 11; 12; | Mela(palm + 33AA)- | melanopsin | OPN1MW | NR(K)Q = NRQ |
| | m | OPN1MW(IL1, IL2, IL3, CT) | | | upstream of splice site: |
| | | | | | NRQFRN/CILHLF |
| G | 13, 14; | OPN1MW(palm)-mGluR6 | OPN1MW | mGluR6 | NR(K)Q = HPE |
| | m | | | | |
| H | 15; 16; | Mela(palm + 33AA)-mGluR6 | melanopsin | mGluR6 | NR(K)Q = HPE |
| | h | | | | |
| I | 17; 18; | Mela(palm)-mGluR6 | melanopsin | mGluR6 | NR(K)Q = HPE |
| | h | L isoform of h melanopsin | | | |
| | | comprising a Golgi export | | | |
| | | signal and a 1D4 tag | | | |
| I | 19; 20; | Mela(palm)-mGluR6 | melanopsin | mGluR6 | NR(K)Q = HPE |
| | h | P isoform of h melanopsin | | | |
| | | comprising a Golgi export | | | |
| | | signal and a 1D4 tag | | | |
| I | 21; 22 | Mela(palm)-mGluR6 | melanopsin | mGluR6 | NR(K)Q = HPE |
| | h | L isoform of h melanopsin | | | |
| | | comprising a Golgi export | | | |
| | | signal | | | |
| I | 23; 24; | Mela(palm)-mGluR6 | melanopsin | mGluR6 | NR(K)Q = HPE |
| | h | P isoform of h melanopsin | | | |
| | | comprising a Golgi export | | | |
| | | signal | | | |
| I | 25; 26; | Mela(palm)-mGluR6 | melanopsin | mGluR6 | NR(K)Q = HPE |
| | h | L isoform of h melanopsin | | | |
| | | without optional C- | | | |
| | | terminally added sequences | | | |
| I | 27; 28; | Mela(palm)-mGluR6 | melanopsin | mGluR6 | NR(K)Q = HPE |
| | h | P isoform of h melanopsin | | | |
| | | without optional C- | | | |
| | | terminally added sequences | | | |
| J | 29; 30; | Mela(palm + 33AA) - | melanopsin | mGluR6 | NR(K)Q = HPE |
| | h | mGluR6(IL1, CT) | | | |
| K | 31; 32; | Mela(palm + 33AA) -mGluR6 | melanopsin | mGluR6 | NR(K)Q = HPE |
| | h | (IL3, CT) | | | |
| L | 33; 34; | JellyOP(palm)-mGluR6 | box jelly fish | mGluR6 | NR(K)Q = HPE |
| | jf/m | | opsin | | |
| M | 35; 36; | JellyOP(palm)-5-HT7 | box jelly fish | 5-HT-7 | QYRNIN = |
| | jf/m | | opsin | | palmitoylation site |
| | | | | | NR(K)Q = HKQ |
| N | 37; 38 | PPO(palm)-mGluR6 | parapinopsin | mGluR6 | NR(K)Q = HPE |
| | ppo/m | | | | |
| O | 39; 40; | JSR1(palm)-mGluR6 | jumping spider | mGluR6 | NR(K)Q = HPE |
| | js/m | | rhodopsin | | |
| P | 41/42 | hJSR(S186F)palm-GABAB2-TS- | hJSR(S186F)palm | GABAB2 | NR(K)Q = NPD |
| | h-JS/m | 1D4 | | | |
| Q | 43/44 | hJSR(S186F)palm-beta2AR | JSR(S186F)palm | beta2AR | NR(K)Q = IAFQ |
| | h-JS/m | | | | |

¹⁾ Odd: nucleic acid sequence; even: amino acid sequence.
²⁾Origin: m = murine; h = human; jf = jelly fish; js = jumping spider; h-js = jumanized jumping spider
³⁾ Name indicates parent GPCRs and the truncation site in the CT of the upstream opsin by referring to a conserved motif or site as in ⁴⁾, regarding the target GPCR, domains are listed unless it is the CT only.
⁴⁾ At or relative to a conserved motif or site; palm = palmitoylation site; palm + 33AA = 33 amino acids distal to a palmitoylation site

In the amino acid sequences listed below framed amino acids refer to conserved motifs and a grey highlight refers to a palmitoylated Cys.

Embodiment (A): Mela (palm + 33AA) -mGluR6 (based on murine
sequences)
Construction: Melanopsin was truncated after AA397 and
the mGluR6 C-terminus was added starting at the NR (K) Q
(SEQ ID NO: 82)/HPE motif.
cDNA- SEQ ID NO 1
atggactctccttcaggaccaagagtcttgtcaagcttaactcaggatcccagcttcacaaccagtcctgccctgcaaggcatttggaacg
gcactcagaacgtctccgtaagagcccagcttctctctgttagccccacgacatctgcacatcaggctgctgcctgggtccccttccccaca
gtcgatgtcccagaccatgctcactataccctaggcacggtgatcctgctggtgggactcacagggatgctgggcaatctgacggtcatct
acaccttctgcaggaacagaggcctgcggacaccagcaaacatgttcatcatcaacctcgcagtcagcgacttcctcatgtcagtcactc
aggccccggtcttctttgccagcagcctctacaagaagtggctctttggggagacaggttgcgagttctatgccttctgcggggctgtcttt
ggcatcacttccatgatcaccctgacagccatagccatggaccgctatctggtgatcacacgtccactggccaccatcggcaggggatcc
aaaagacgaacggcactcgtcctgctaggcgtctggctttatgccctggcctggagtctgccacctttctttggttggagtgcctacgtgcc
cgaggggctgctgacatcctgctcctgggactacatgaccttcacaccccaggtgcgtgcctacaccatgctgctcttctgctttgtcttctt
cctccccctgctcatcatcatcttctgctacatcttcatcttcagggccatccgagagacaggccgggcctgtgagggctgcggtgagtccc
ctctgcggcagaggcggcagtggcagcggctgcagagtgagtggaagatggccaaggtcgcactgattgtcattcttctcttcgtgctgtc
ctgggctccctactccactgtggctctggtggcctttgctggatactcgcacatcctgacgccctacatgagctcggtgccagccgtcatcg
ccaaggcttctgccatccacaatcccattatctacgccatcactcaccccaagtacagggtggccattgcccagcacctgccttgccttgg
ggtgcttctcggtgtatcaggccagcgcagccacccctccctcagctaccgctctacccaccgctccacattgagcagccagtcctcagac
ctccatccagagcagaacgtgcagaagcggaagcgcagcctcaagaagacctccacgatggcggccccgcccaagagcgagaactca
gaggacgccaagaagagcaggatcaccagcgagggcgagtacatccccctggaccagatcgacatcaacgtgacagagaccagcca
agtggcgcctgcctaa
Peptide sequence- SEQ ID NO 2
MDSPSGPRVLSSLTQDPSFTTSPALQGIWNGTQNVSVRAQLLSVSPTTSAHQAAAWVPFPTVDVPDHAHY
TLGTVILLVGLTGMLGNLTVIYTFCRNRGLRTPANMFIINLAVSDFLMSVTQAPVFFASSLYKKWLFGETGCEF
YAFCGAVFGITSMITLTAIAMDRYLVITRPLATIGRGSKRRTALVLLGVWLYALAWSLPPFFGWSAYVPEGLLT
SCSWDYMTFTPQVRAYTMLLFCFVFFLPLLIIIFCYIFIFRAIRETGRACEGCGESPLRQRRQWQRLQSEWKMA

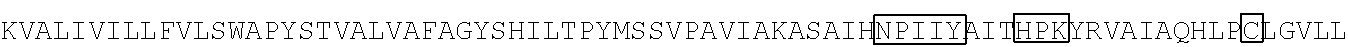

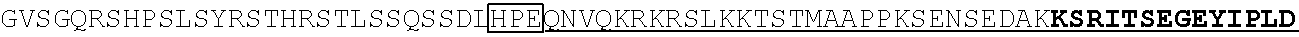

QIDINVTETSQVAPA
Legend:
Underlined = GRM6
Underlined and bold = optional Golgi export signal
Bold = optional 1D4 epitope
Embodiment (B): Mela (palm) -mGluR6 (based on murine
sequences)
Construction: Melanopsin was truncated after the
palmitoylated Cys of melanopsin (AA364) and the mGluR6 C-
terminus added including two additional proximal amino
acids as a consequence of better sequence alignment
compared to prototype Opto-mGluR6 (van Wyk M et al.,
2015).
cDNA- SEQ ID NO 3
atggactctccttcaggaccaagagtcttgtcaagcttaactcaggatcccagcttcacaaccagtcctgccctgcaaggcatttggaacg
gcactcagaacgtctccgtaagagcccagcttctctctgttagccccacgacatctgcacatcaggctgctgcctgggtccccttccccaca
gtcgatgtcccagaccatgctcactataccctaggcacggtgatcctgctggtgggactcacagggatgctgggcaatctgacggtcatct
acaccttctgcaggaacagaggcctgcggacaccagcaaacatgttcatcatcaacctcgcagtcagcgacttcctcatgtcagtcactc
aggccccggtcttctttgccagcagcctctacaagaagtggctctttggggagacaggttgcgagttctatgccttctgcggggctgtcttt
ggcatcacttccatgatcaccctgacagccatagccatggaccgctatctggtgatcacacgtccactggccaccatcggcaggggatcc
aaaagacgaacggcactcgtcctgctaggcgtctggctttatgccctggcctggagtctgccacctttctttggttggagtgcctacgtgcc
cgaggggctgctgacatcctgctcctgggactacatgaccttcacaccccaggtgcgtgcctacaccatgctgctcttctgctttgtcttctt
cctccccctgctcatcatcatcttctgctacatcttcatcttcagggccatccgagagacaggccgggcctgtgagggctgcggtgagtccc
ctctgcggcagaggcggcagtggcagcggctgcagagtgagtggaagatggccaaggtcgcactgattgtcattcttctcttcgtgctgtc
ctgggctccctactccactgtggctctggtggcctttgctggatactcgcacatcctgacgccctacatgagctcggtgccagccgtcatcg
ccaaggcttctgccatccacaatcccattatctacgccatcactcaccccaagtacagggtggccattgcccagcacctgccttgcctgttc
catccagagcagaacgtgcagaagcggaagcgcagcctcaagaagacctccacgatggcggccccgcccaagagcgagaactcaga
ggacgccaagaagagcaggatcaccagcgagggcgagtacatccccctggaccagatcgacatcaacgtgacagagaccagccaag
tggcgcctgcctaa
Peptide sequence- SEQ ID NO 4
MDSPSGPRVLSSLTQDPSFTTSPALQGIWNGTQNVSVRAQLLSVSPTTSAHQAAAWVPFPTVDVPDHAHY
TLGTVILLVGLTGMLGNLTVIYTFCRNRGLRTPANMFIINLAVSDFLMSVTQAPVFFASSLYKKWLFGETGCEF
YAFCGAVFGITSMITLTAIAMDRYLVITRPLATIGRGSKRRTALVLLGVWLYALAWSLPPFFGWSAYVPEGLLT
SCSWDYMTFTPQVRAYTMLLFCFVFFLPLLIIIFCYIFIFRAIRETGRACEGCGESPLRQRRQWQRLQSEWKMA

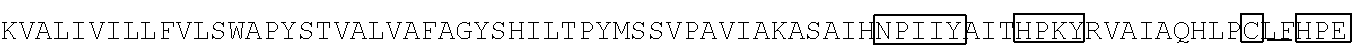

QNVQKRKRSLKKTSTMAAPPKSENSEDAKKSRITSEGEYIPLDQIDINVTETSQVAPA
Legend:
Underlined = GRM6
Underlined and bold = optional Golgi export signal
Bold = optional 1D4 epitope
Embodiment (C): Mela (palm + 33AA) + IL1-mGluR6 (based on
murine sequences)
Construction: As Embodiment A, but with additional
complete exchange of melanopsin IL1 with IL1 of mGluR6.
Identical cutting sites as in prototype Opto-mGluR6 (van
Wyk M et al., 2015).
cDNA- SEQ No. 5
atggactctccttcaggaccaagagtcttgtcaagcttaactcaggatcccagcttcacaaccagtcctgccctgcaaggcatttggaacg
gcactcagaacgtctccgtaagagcccagcttctctctgttagccccacgacatctgcacatcaggctgctgcctgggtccccttccccaca
gtcgatgtcccagaccatgctcactataccctaggcacggtgatcctgctggtgggactcacagggatgctgggcaatctgacggtcatct
acaccttcatgcgacacaacgacactcccatagtccgcgcctctggccgtgagcttttcatcatcaacctcgcagtcagcgacttcctcatg
tcagtcactcaggccccggtcttctttgccagcagcctctacaagaagtggctctttggggagacaggttgcgagttctatgccttctgcgg
ggctgtctttggcatcacttccatgatcaccctgacagccatagccatggaccgctatctggtgatcacacgtccactggccaccatcggc
aggggatccaaaagacgaacggcactcgtcctgctaggcgtctggctttatgccctggcctggagtctgccacctttctttggttggagtgc
ctacgtgcccgaggggctgctgacatcctgctcctgggactacatgaccttcacaccccaggtgcgtgcctacaccatgctgctcttctgct
ttgtcttcttcctccccctgctcatcatcatcttctgctacatcttcatcttcagggccatccgagagacaggccgggcctgtgagggctgcg
gtgagtcccctctgcggcagaggcggcagtggcagcggctgcagagtgagtggaagatggccaaggtcgcactgattgtcattcttctct
tcgtgctgtcctgggctccctactccactgtggctctggtggcctttgctggatactcgcacatcctgacgccctacatgagctcggtgccag
ccgtcatcgccaaggcttctgccatccacaatcccattatctacgccatcactcaccccaagtacagggtggccattgcccagcacctgcc
ttgccttggggtgcttctcggtgtatcaggccagcgcagccacccctccctcagctaccgctctacccaccgctccacattgagcagccagt
cctcagacctccatccagagcagaacgtgcagaagcggaagcgcagcctcaagaagacctccacgatggggccccgcccaagagcg
agaactcagaggacgccaagaagagcaggatcaccagcgagggcgagtacatccccctggaccagatcgacatcaacgtgacagag
accagccaagtggcgcctgcctaa
Peptide sequence- SEQ No. 6
MDSPSGPRVLSSLTQDPSFTTSPALQGIWNGTQNVSVRAQLLSVSPTTSAHQAAAWVPFPTVDVPDHAHY
TLGTVILLVGLTGMLGNLTVIYTFMRHNDTPIVRASGRELFIINLAVSDFLMSVTQAPVFFASSLYKKWLFGET

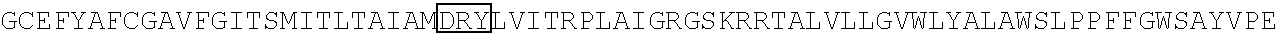

GLLTSCSWDYMTFTPQVRAYTMLLFCFVFFLPLLIIIFCYIFIFRAIRETGRACEGCGESPLRQRRQWQRLQSEW

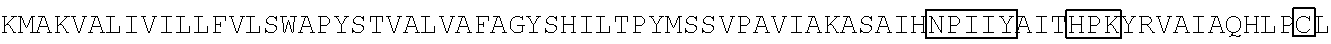

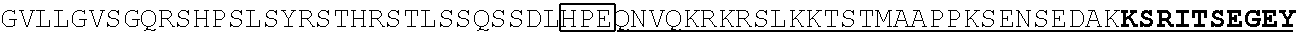

IPLDQIDINVTETSQVAPA
Legend:
Underlined = GRM6
Underlined and bold = optional Golgi export signal
Bold = optional 1D4 epitope
Embodiment (D): Mela (palm) + IL1-mGluR6 (based on murine
sequences)
Construction: Melanopsin was truncated after the
palmitoylated Cys of melanopsin (AA364) and the mGluR6 C-
terminus added including two additional proximal AA
compared to prototype Opto-mGluR6 (van Wyk M et al.,
2015).
cDNA- SEQ No. 7
atggactctccttcaggaccaagagtcttgtcaagcttaactcaggatcccagcttcacaaccagtcctgccctgcaaggcatttggaacg
gcactcagaacgtctccgtaagagcccagcttctctctgttagccccacgacatctgcacatcaggctgctgcctgggtccccttccccaca
gtcgatgtcccagaccatgctcactataccctaggcacggtgatcctgctggtgggactcacagggatgctgggcaatctgacggtcatct
acaccttctgcaggaacagaggcctgcggacaccagcaaacatgttcatcatcaacctcgcagtcagcgacttcctcatgtcagtcactc
aggccccggtcttctttgccagcagcctctacaagaagtggctctttggggagacaggttgcgagttctatgccttctgcggggctgtcttt
ggcatcacttccatgatcaccctgacagccatagccatggaccgctatctggtgatcacacgtccactggccaccatcggcaggggatcc
aaaagacgaacggcactcgtcctgctaggcgtctggctttatgccctggcctggagtctgccacctttctttggttggagtgcctacgtgcc
cgaggggctgctgacatcctgctcctgggactacatgaccttcacaccccaggtgcgtgcctacaccatgctgctcttctgctttgtcttctt
cctccccctgctcatcatcatcttctgctacatcttcatcttcagggccatccgagagacaggccgggcctgtgagggctgcggtgagtccc
ctctgcggcagaggcggcagtggcagcggctgcagagtgagtggaagatggccaaggtcgcactgattgtcattcttctcttcgtgctgtc
ctgggctccctactccactgtggctctggtggcctttgctggatactcgcacatcctgacgccctacatgagctcggtgccagccgtcatcg
ccaaggcttctgccatccacaatcccattatctacgccatcactcaccccaagtacagggtggccattgcccagcacctgccttgcctgttc
catccagagcagaacgtgcagaagcggaagcgcagcctcaagaagacctccacgatggcggccccgcccaagagcgagaactcaga
ggacgccaagaagagcaggatcaccagcgagggcgagtacatccccctggaccagatcgacatcaacgtgacagagaccagccaag
tggcgcctgcctaa
Peptide sequence- SEQ No. 8
MDSPSGPRVLSSLTQDPSFTTSPALQGIWNGTQNVSVRAQLLSVSPTTSAHQAAAWVPFPTVDVPDHAHY
TLGTVILLVGLTGMLGNLTVIYTFMRHNDTPIVRASGRELFIINLAVSDFLMSVTQAPVFFASSLYKKWLFGET
GCEFYAFCGAVFGITSMITLTAIAMDRYLVITRPLATIGRGSKRRTALVLLGVWLYALAWSLPPFFGWSAYVPE
GLLTSCSWDYMTFTPQVRAYTMLLFCFVFFLPLLIIIFCYIFIFRAIRETGRACEGCGESPLRQRRQWQRLQSEW

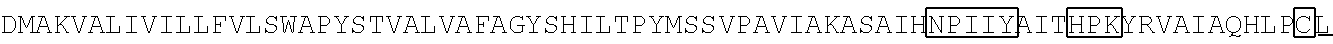

FHPEQNVQKRKRSLKKTSTMAAPPKSENSEDAKKSRITSEGEYIPLDQIDINVTETSQVAPA
Legend:
Underlined = GRM6
Underlined and bold = optional Golgi export signal
Bold = optional 1D4 epitope
Embodiment (E): Mela (palm + 33AA) + IL3-mGluR6 (based on
murine sequences)
Construction: As (1), but with additional insertion of
the short IL3 of mGluR6 into the variable portion of the
long IL3 of melanopsin.
cDNA- SEQ No. 9
atggactctccttcaggaccaagagtcttgtcaagcttaactcaggatcccagcttcacaaccagtcctgccctgcaaggcatttggaacg
gcactcagaacgtctccgtaagagcccagcttctctctgttagccccacgacatctgcacatcaggctgctgcctgggtccccttccccaca
gtcgatgtcccagaccatgctcactataccctaggcacggtgatcctgctggtgggactcacagggatgctgggcaatctgacggtcatct
acaccttctgcaggaacagaggcctgcggacaccagcaaacatgttcatcatcaacctcgcagtcagcgacttcctcatgtcagtcactc
aggccccggtcttctttgccagcagcctctacaagaagtggctctttggggagacaggttgcgagttctatgccttctgcggggctgtcttt
ggcatcacttccatgatcaccctgacagccatagccatggaccgctatctggtgatcacacgtccactggccaccatcggcaggggatcc
aaaagacgaacggcactcgtcctgctaggcgtctggctttatgccctggcctggagtctgccacctttctttggttggagtgcctacgtgcc
cgaggggctgctgacatcctgctcctgggactacatgaccttcacaccccaggtgcgtgcctacaccatgctgctcttctgctttgtcttctt
cctccccctgctcatcatcatcttctgctacatcttcatcttcagggccatccgagagacaggccggggtgtgccagagaccttcaatgaag
cccctctgcggcagaggcggcagtggcagcggctgcagagtgagtggaagatggccaaggtcgcactgattgtcattcttctcttcgtgct
gtcctgggctccctactccactgtggctctggtggcctttgctggatactcgcacatcctgacgccctacatgagctcggtgccagccgtca
tcgccaaggcttctgccatccacaatcccattatctacgccatcactcaccccaagtacagggggccattgcccagcacctgccttgcctt
ggggtgcttctcggtgtatcaggccagcgcagccacccctccctcagctaccgctctacccaccgctccacattgagcagccagtcctcag
acctccatccagagcagaacgtgcagaagcggaagcgcagcctcaagaagacctccacgatggcggccccgcccaagagcgagaact
cagaggacgccaagaagagcaggatcaccagcgagggcgagtacatccccctggaccagatcgacatcaacgtgacagagaccagc
caagtggcgcctgcctaa
Peptide sequence- SEQ No. 10
MDSPSGPRVLSSLTQDPSFTTSPALQGIWNGTQNVSVRAQLLSVSPTTSAHQAAAWVPFPTVDVPDHAHY
TLGTVILLVGLTGMLGNLTVIYTFCRNRGLRTPANMFIINLAVSDFLMSVTQAPVFFASSLYKKWLFGETGCEF
YAFCGAVFGITSMITLTAIAMDRYLVITRPLATIGRGSKRRTALVLLGVWLYALAWSLPPFFGWSAYVPEGLLT
SCSWDYMTFTPQVRAYTMLLFCFVFFLPLLIIIFCYIFIFRAIRETGRGVPETFNEAPLRQRRQWQRLQSEWKM

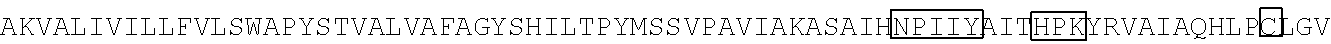

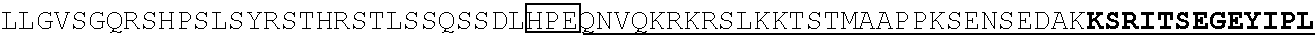

DQIDINVTETSQVAPA
Legend:
Underlined = GRM6
Underlined and bold = optional Golgi export signal
Bold = optional 1D4 epitope
Embodiment (F): Mela (palm) -OPN1MW (IL1, IL2, IL3, CT)
(based on murine sequences)
Construction:
chimera with all intracellular domains of mela exchanged
and by corresponding intracellular domains of OPN1MW with
CT splice site X-1 (FIG. 3 option 1)
cDNA- SEQ No. 11
atggactctccttcaggaccaagagtcttgtcaagcttaactcaggatcccagcttcacaaccagtcctgccctgcaaggcatttggaacg
gcactcagaacgtctccgtaagagcccagcttctctctgttagccccacgacatctgcacatcaggctgctgcctgggtccccttccccaca
gtcgatgtcccagaccatgctcactataccctaggcacggtgatcctgctggtgggactcacagggatgctgggcaatctgacggtcatct
acgccaccatgagattcaagaagctgcgccatccagcaaacatgttcatcatcaacctcgcagtcagcgacttcctcatgtcagtcactca
ggccccggtcttctttgccagcagcctctacaagaagtggctctttggggagacaggttgcgagttctatgccttctgcggggctgtctttg
gcatcacttccatgatcaccctgacagccatagccatggaccgctatctggtgatctgcaagccctttggcaatgtgagatttgatgctaa
gctgacggcactcgtcctgctaggcgtctggctttatgccctggcctggagtctgccacctttctttggttggagtgcctacgtgcccgaggg
gctgctgacatcctgctcctgggactacatgaccttcacaccccaggtgcgtgcctacaccatgctgctcttctgctttgtcttcttcctcccc
ctgctcatcatcatcttctgctacatccaagtgtggctggccatccgagcagtggcaaagcaacagaaagaatctgagtccactcagaag
gccgagaaggaggtgacaaaggtcgcactgattgtcattcttctcttcgtgctgtcctgggctccctactccactgtggctctggtggccttt
gctggatactcgcacatcctgacgccctacatgagctcggtgccagccgtcatcgccaaggcttctgccatccacaatcccattatctacg
ccatcactcaccccaagtacagggtggccattgcccagcacctgccttgcaactgcatcttacatctctttggaaagaaggttgatgatag
ctctgaactttccagcacctccaagacagaagtctcatctgtctcttcagtgtcacctgcataa
Peptide sequence- SEQ No. 12
MDSPSGPRVLSSLTQDPSFTTSPALQGIWNGTQNVSVRAQLLSVSPTTSAHQAAAWVPFPTVDVPDHAHY
TLGTVILLVGLTGMLGNLTVIYATMRFKKLRHPANMFIINLAVSDFLMSVTQAPVFFASSLYKKWLFGETGCE

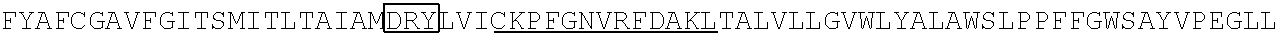

TSCSWDYMTFTPQVRAYTMLLFCFVFFLPLLIIIFCYIQVWLAIRAVAKQQKESESTQKAEKEVTKVALIVILLFV

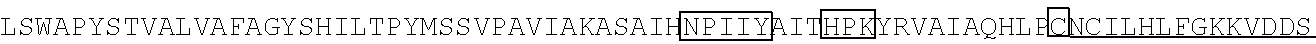

SELSSTSKTEVSSVSSVSPA
Legend:
Underlined = OPN1MW
Underlined and bold = optional Golgi export signal
Bold = optional 1D4 epitope
Embodiment (G): OPN1MW (palm) -mGluR6 (based on murine
sequences)
Construction: The mGluR6 C-terminus including the HPE
motif was added at the putative palmitoylation site, i.e.
behind the residue (F, highlighted in grey) just before
the palmitoylation site C322 in bovine rhodopsin.
cDNA- SEQ No. 13
atggcccaaaggcttacaggtgaacagacactggaccactatgaggatagcacccatgcaagcatcttcacctataccaacagcaaca
gcaccaaaggtccctttgaaggccccaattatcacattgctcccaggtgggtgtaccacctcaccagcacctggatgattcttgtggtcgtt
gcatctgtcttcactaatggacttgtgctggcagccaccatgagattcaagaagctgcgccatccactgaactggattctggtgaacttgg
cagttgctgacctagcagagaccattattgccagcactatcagtgttgtgaaccaaatctatggctacttcgttctgggacaccctctgtgt
gtcattgaaggctacattgtctcattgtgtggaatcacaggcctctggtccctggccatcatttcctgggagagatggctggtggtctgcaa
gccctttggcaatgtgagatttgatgctaagctggccactgtgggaatcgtcttctcctgggtctgggctgctatatggacggccccaccaa
tctttggttggagcaggtactggccttatggcctgaagacatcctgtggcccagacgtgttcagcggtacctcgtaccccggggttcagtct
tatatgatggtcctcatggtcacgtgctgcatcttcccactcagcatcatcgtgctctgctacctccaagtgtggctggccatccgagcagtg
gcaaagcaacagaaagaatctgagtccactcagaaggccgagaaggaggtgacacgcatggtggtggtgatggtcttcgcatactgcc
tctgctggggaccctatactttctttgcatgctttgctactgcccaccctggctatgccttccaccctcttgtggcctccctaccatcctacttt
gccaaaagtgccactatctacaaccccattatctatgtctttatgaaccggcagtttcgaaactgcatcttacatctctttcatccagagca
gaacgtgcagaagcggaagcgcagcctcaagaagacctccacgatggcggccccgcccaagagcgagaactcagaggacgccaag
Peptide sequence- SEQ No. 14
MAQRLTGEQTLDHYEDSTHASIFTYTNSNSTKGPFEGPNYHIAPRWVYHLTSTWMILVVVASVFTNGLVLAA
TMRFKKLRHPLNWILVNLAVADLAETIIASTISVVNQIYGYFVLGHPLCVIEGYIVSLCGITGLWSLAIISWERWL
VVCKPFGNVRFDAKLATVGIVFSWVWAAIWTAPPIFGWSRYWPYGLKTSCGPDVFSGTSYPGVQSYMMVL
MVTCCIFPLSIIVLCYLQVWLAIRAVAKQQKESESTQKAEKEVTRMVVVMVFAYCLCWGPYTFFACFATAHP

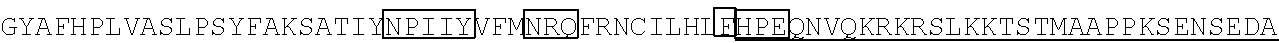

KKSRITSEGEYIPLDQIDINVTETSQVAPA
Legend:
Underlined = mGluR6
Underlined and bold = optional Golgi export signal
Bold = optional 1D4 epitope
Embodiment (H): Mela (palm + 33AA) -mGluR6 (based on human
sequences)
Construction: as above with murine construct
cDNA- SEQ No. 15
atgaaccctccttcggggccaagagtcctgcccagcccaacccaagagcccagctgcatggccaccccagcaccacccagctggtggga
cagctcccagagcagcatctccagcctgggccggcttccatccatcagtcccacagcacctgggacttgggctgctgcctgggtccccctc
cccacggttgatgttccagaccatgcccactataccctgggcacagtgatcttgctggtgggactcacggggatgctgggcaacctgacg
gtcatctataccttctgcaggagcagaagcctccggacacctgccaacatgttcattatcaacctcgcggtcagcgacttcctcatgtcctt
cacccaggcccctgtcttcttcaccagtagcctctataagcagtggctctttggggagacaggctgcgagttctatgccttctgtggagctc
tctttggcatttcctccatgatcaccctgacggccatcgccctggaccgctacctggtaatcacacgcccgctggccacctttggtgtggcgt
ccaagaggcgtgcggcatttgtcctgctgggcgtttggctctatgccctggcctggagtctgccacccttcttcggctggagcgcctacgtg
cccgaggggttgctgacatcctgctcctgggactacatgagcttcacgccggccgtgcgtgcctacaccatgcttctctgctgcttcgtgttc
ttcctccctctgcttatcatcatctactgctacatcttcatcttcagggccatccgggagacaggacgggctctccagaccttcggggcctgc
aagggcaatggcgagtccctgtggcagcggcagcggctgcagagcgagtgcaagatggccaagatcatgctgctggtcatcctcctctt
cgtgctctcctgggctccctattccgctgtggccctggtggcctttgctgggtacgcacacgtcctgacaccctacatgagctcggtgccag
ccgtcatcgccaaggcctctgcaatccacaaccccatcatttacgccatcacccaccccaagtacagggtggccattgcccagcacctgc
cctgcctgggggtgctgctgggtgtatcacgccggcacagtcgcccctaccccagctaccgctccacccaccgctccacgctgaccagcc
acacctccaacctccatccagagcagaatgtgcagaagcgaaagcggagcctcaaggccacctccacggtggcagccccacccaaggg
cgaggatgcagaggcccacaagaagagcaggatcaccagcgagggcgagtacatccccctggaccagatcgacatcaacgtgacag
agaccagccaagtggcgcctgcctaa
Peptide sequence- SEQ No. 16
MNPPSGPRVLPSPTQEPSCMATPAPPSWWDSSQSSISSLGRLPSISPTAPGTWAAAWVPLPTVDVPDHAH
YTLGTVILLVGLTGMLGNLTVIYTFCRSRSLRTPANMFIINLAVSDFLMSFTQAPVFFTSSLYKQWLFGETGCEF
YAFCGALFGISSMITLTAIALDRYLVITRPLATFGVASKRRAAFVLLGVWLYALAWSLPPFFGWSAYVPEGLLTS
CSWDYMSFTPAVRAYTMLLCCFVFFLPLLIIIYCYIFIFRAIRETGRALQTFGACKGNGESLWQRQRLQSECKM

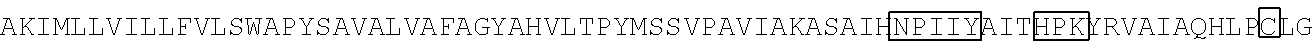

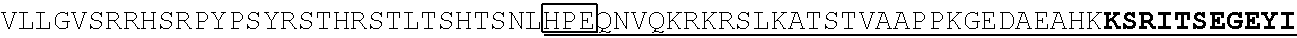

PLDQIDINVTETSQVAPA
Legend:
Underlined = GRM6
Underlined and bold = optional Golgi export signal
Bold = optional 1D4 epitope
Embodiment (I): Mela (palm) -mGluR6 (based on human
sequences)
Construction: as above with murine construct
Allelic variant: “L variant”
cDNA- SEQ No. 17

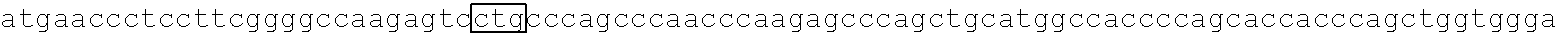

cagctcccagagcagcatctccagcctgggccggcttccatccatcagtcccacagcacctgggacttgggctgctgcctgggtccccctc
cccacggttgatgttccagaccatgcccactataccctgggcacagtgatcttgctggtgggactcacggggatgctgggcaacctgacg
gtcatctataccttctgcaggagcagaagcctccggacacctgccaacatgttcattatcaacctcgcggtcagcgacttcctcatgtcctt
cacccaggcccctgtcttcttcaccagtagcctctataagcagtggctctttggggagacaggctgcgagttctatgccttctgtggagctc
tctttggcatttcctccatgatcaccctgacggccatcgccctggaccgctacctggtaatcacacgcccgctggccacctttggtgtggcgt
ccaagaggcgtgcggcatttgtcctgctgggcgtttggctctatgccctggcctggagtctgccacccttcttcggctggagcgcctacgtg
cccgaggggttgctgacatcctgctcctgggactacatgagcttcacgccggccgtgcgtgcctacaccatgcttctctgctgcttcgtgttc
ttcctccctctgcttatcatcatctactgctacatcttcatcttcagggccatccgggagacaggacgggctctccagaccttcggggcctgc
aagggcaatggcgagtccctgtggcagcggcagcggctgcagagcgagtgcaagatggccaagatcatgctgctggtcatcctcctctt
cgtgctctcctgggctccctattccgctgtggccctggtggcctttgctgggtacgcacacgtcctgacaccctacatgagctcggtgccag
ccgtcatcgccaaggcctctgcaatccacaaccccatcatttacgccatcacccaccccaagtacagggtggccattgcccagcacctgc
cctgcctgttccatccagagcagaatgtgcagaagcgaaagcggagcctcaaggccacctccacggtggcagccccacccaagggcga
ggatgcagaggcccacaagaagagcaggatcaccagcgagggcgagtacatccccctggaccagatcgacatcaacgtgacagaga
ccagccaagtggcgcctgcctaa
Peptide sequence- SEQ No. 18

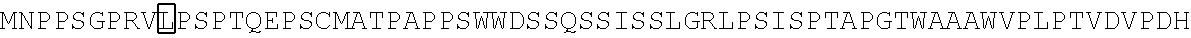

AHYTLGTVILLVGLTGMLGNLTVIYTFCRSRSLRTPANMFIINLAVSDFLMSFTQAPVFFTSSLYKQWLFGE
TGCEFYAFCGALFGISSMITLTAIALDRYLVITRPLATFGVASKRRAAFVLLGVWLYALAWSLPPFFGWSAY
VPEGLLTSCSWDYMSFTPAVRAYTMLLCCFVFFLPLLIIIYCYIFIFRAIRETGRALQTFGACKGNGESLWQR

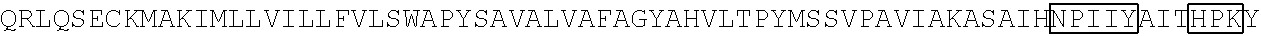

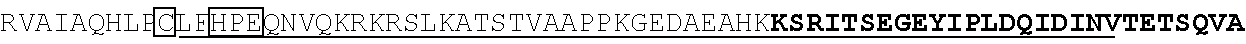

PA
Allelic variant: P-variant
cDNA SEQ No. 19 (with human melanopsin isoform 1)

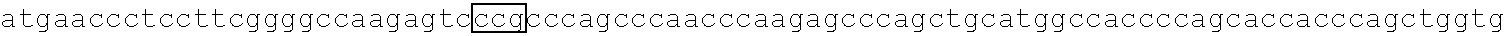

ggacagctcccagagcagcatctccagcctgggccggcttccatccatcagtcccacagcacctgggacttgggctgctgcctgggtc
cccctccccacggttgatgttccagaccatgcccactataccctgggcacagtgatcttgctggtgggactcacggggatgctgggca
acctgacggtcatctataccttctgcaggagcagaagcctccggacacctgccaacatgttcattatcaacctcgcggtcagcgactt
cctcatgtccttcacccaggcccctgtcttcttcaccagtagcctctataagcagtggctctttggggagacaggctgcgagttctatgc
cttctgtggagctctctttggcatttcctccatgatcaccctgacggccatcgccctggaccgctacctggtaatcacacgcccgctggc
cacctttggtgtggcgtccaagaggcgtgcggcatttgtcctgctgggcgtttggctctatgccctggcctggagtctgccacccttctt
cggctggagcgcctacgtgcccgaggggttgctgacatcctgctcctgggactacatgagcttcacgccggccgtgcgtgcctacacc
atgcttctctgctgcttcgtgttcttcctccctctgcttatcatcatctactgctacatcttcatcttcagggccatccgggagacaggacg
ggctctccagaccttcggggcctgcaagggcaatggcgagtccctgtggcagcggcagcggctgcagagcgagtgcaagatggcca
agatcatgctgctggtcatcctcctcttcgtgctctcctgggctccctattccgctgtggccctggtggcctttgctgggtacgcacacgt
cctgacaccctacatgagctcggtgccagccgtcatcgccaaggcctctgcaatccacaaccccatcatttacgccatcacccacccc
aagtacagggtggccattgcccagcacctgccctgcctgttccatccagagcagaatgtgcagaagcgaaagcggagcctcaaggc
cacctccacggtggcagccccacccaagggcgaggatgcagaggcccacaagaagagcaggatcaccagcgagggcgagtaca
tccccctggaccagatcgacatcaacgtgacagagaccagccaagtggcgcctgcctaa
Peptide sequence- SEQ No. 20
AA sequence melanopsin-mGluR6) with isoform 1 of human
melanopsin (“p variant”)

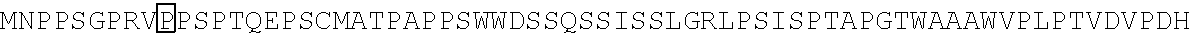

AHYTLGTVILLVGLTGMLGNLTVIYTFCRSRSLRTPANMFIINLAVSDFLMSFTQAPVFFTSSLYKQWLFGE
TGCEFYAFCGALFGISSMITLTAIALDRYLVITRPLATFGVASKRRAAFVLLGVWLYALAWSLPPFFGWSAY
VPEGLLTSCSWDYMSFTPAVRAYTMLLCCFVFFLPLLIIIYCYIFIFRAIRETGRALQTFGACKGNGESLWQR

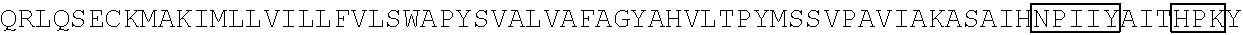

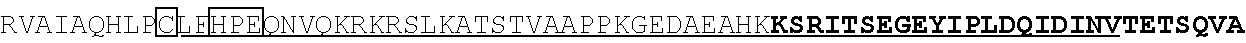

PA
Legend:
Underlined = GRM6
Underlined and bold = optional Golgi export signal
Bold = optional 1D4 epitope
Framed = Allelic variant and conserved motifs
Comment: In comparative experiments in HEK293 cells the L
and P variants performed equally. The P-variant is the
most common mGluR6 allelic variant and it has been used
in most experiments.
Embodiment (J): Mela (palm + 33A) + IL1-mGluR6 (based on human
sequences)
Construction: as above with murine construct
cDNA- SEQ No. 29
atgaaccctccttcggggccaagagtcctgcccagcccaacccaagagcccagctgcatggccaccccagcaccacccagctggtggga
cagctcccagagcagcatctccagcctgggccggcttccatccatcagtcccacagcacctgggacttgggctgctgcctgggtccccctc
cccacggttgatgttccagaccatgcccactataccctgggcacagtgatcttgctggtgggactcacggggatgctgggcaacctgacg
gtcatctataccttccggtacaacaacacgcccatcgtccgggcctcgggccgagagctcttcattatcaacctcgcggtcagcgacttcct
catgtccttcacccaggcccctgtcttcttcaccagtagcctctataagcagtggctctttggggagacaggctgcgagttctatgccttctg
tggagctctctttggcatttcctccatgatcaccctgacggccatcgccctggaccgctacctggtaatcacacgcccgctggccacctttg
gtgtggcgtccaagaggcgtgcggcatttgtcctgctgggcgtttggctctatgccctggcctggagtctgccacccttcttcggctggagc
gcctacgtgcccgaggggttgctgacatcctgctcctgggactacatgagcttcacgccggccgtgcgtgcctacaccatgcttctctgctg
cttcgtgttcttcctccctctgcttatcatcatctactgctacatcttcatcttcagggccatccgggagacaggacgggctctccagaccttc
ggggcctgcaagggcaatggcgagtccctgtggcagcggcagcggctgcagagcgagtgcaagatggccaagatcatgctgctggtca
tcctcctcttcgtgctctcctgggctccctattccgctgtggccctggtggcctttgctgggtacgcacacgtcctgacaccctacatgagctc
ggtgccagccgtcatcgccaaggcctctgcaatccacaaccccatcatttacgccatcacccaccccaagtacagggtggccattgccca
gcacctgccctgcctgggggtgctgctgggtgtatcacgccggcacagtcgcccctaccccagctaccgctccacccaccgctccacgctg
accagccacacctccaacctccatccagagcagaatgtgcagaagcgaaagcggagcctcaaggccacctccacggtggcagccccac
ccaagggcgaggatgcagaggcccacaagaagagcaggatcaccagcgagggcgagtacatccccctggaccagatcgacatcaac
gtgacagagaccagccaagtggcgcctgcctaa
Peptide sequence- SEQ No. 30
MNPPSGPRVLPSPTQEPSCMATPAPPSWWDSSQSSISSLGRLPSISPTAPGTWAAAWVPLPTVDVPDHAH
YTLGTVILLVGLTGMLGNLTVIYTFRYNNTPIVRASGRELFIINLAVSDFLMSFTQAPVFFTSSLYKQWLFGETG
CEFYAFCGALFGISSMITLTAIALDRYLVITRPLATFGVASKRRAAFVLLGVWLYALAWSLPPFFGWSAYVPEGL
LTSCSWDYMSFTPAVRAYTMLLCCFVFFLPLLIIIYCYIFIFRAIRETGRALQTFGACKGNGESLWQRQRLQSEC

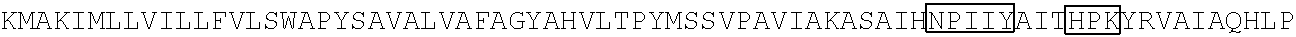

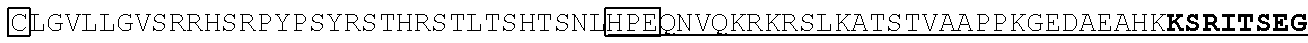

EYIPLDQIDINVTETSQVAPA
Legend:
Underlined = GRM6
Underlined and bold = optional Golgi export signal
Bold = optional 1D4 epitope
Embodiment (K): Mela (palm + 33A) + IL3-mGluR6 (based on human
sequences)
Construction: Human melanopsin chimera with mGluR6 (GRM6)
IL3 and mGluR6 (GRM6) CT
cDNA- SEQ No. 31
atgaaccctccttcggggccaagagtcctgcccagcccaacccaagagcccagctgcatggccaccccagcaccacccagctggtggga
cagctcccagagcagcatctccagcctgggccggcttccatccatcagtcccacagcacctgggacttgggctgctgcctgggtccccctc
cccacggttgatgttccagaccatgcccactataccctgggcacagtgatcttgctggtgggactcacggggatgctgggcaacctgacg
gtcatctataccttctgcaggagcagaagcctccggacacctgccaacatgttcattatcaacctcgcggtcagcgacttcctcatgtcctt
cacccaggcccctgtcttcttcaccagtagcctctataagcagtggctctttggggagacaggctgcgagttctatgccttctgtggagctc
tctttggcatttcctccatgatcaccctgacggccatcgccctggaccgctacctggtaatcacacgcccgctggccacctttggtgtggcgt
ccaagaggcgtgcggcatttgtcctgctgggcgtttggctctatgccctggcctggagtctgccacccttcttcggctggagcgcctacgtg
cccgaggggttgctgacatcctgctcctgggactacatgagcttcacgccggccgtgcgtgcctacaccatgcttctctgctgcttcgtgttc
ttcctccctctgcttatcatcatctactgctacatcttcatcttcagggccatccgggagacaggacggggcgtgcccgagaccttcaacga
ggccaagggcaatggcgagtccctgtggcagcggcagcggctgcagagcgagtgcaagatggccaagatcatgctgctggtcatcctcc
tcttcgtgctctcctgggctccctattccgctgtggccctggtggcctttgctgggtacgcacacgtcctgacaccctacatgagctcggtgc
cagccgtcatcgccaaggcctctgcaatccacaaccccatcatttacgccatcacccaccccaagtacagggtggccattgcccagcacc
tgccctgcctgggggtgctgctgggtgtatcacgccggcacagtcgcccctaccccagctaccgctccacccaccgctccacgctgaccag
ccacacctccaacctccatccagagcagaatgtgcagaagcgaaagcggagcctcaaggccacctccacggtggcagccccacccaag
ggcgaggatgcagaggcccacaagaagagcaggatcaccagcgagggcgagtacatccccctggaccagatcgacatcaacgtgac
agagaccagccaagtggcgcctgcctaa
Peptide sequence- SEQ No. 32
MNPPSGPRVLPSPTQEPSCMATPAPPSWWDSSQSSISSLGRLPSISPTAPGTWAAAWVPLPTVDVPDHAH
YTLGTVILLVGLTGMLGNLTVIYTFCRSRSLRTPANMFIINLAVSDFLMSFTQAPVFFTSSLYKQWLFGETGCEF
YAFCGALFGISSMITLTAIALDRYLVITRPLATFGVASKRRAAFVLLGVWLYALAWSLPPFFGWSAYVPEGLLTS
CSWDYMSFTPAVRAYTMLLCCFVFFLPLLIIIYCYIFIFRAIRETGRGVPETFNEAKGNGESLWQRQRLQSECK
MAKIMLLVILLFVLSWAPYSAVALVAFAGYAHVLTPYMSSVPAVIAKASAIHNPIIYAITHPKYRVAIAQHLPCL

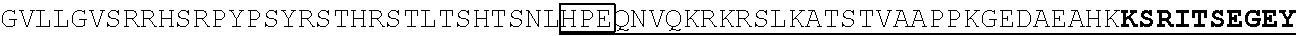

IPLDQIDINVTETSQVAPA
Legend:
Underlined = GRM6
Underlined and bold = optional Golgi export signal
Bold = optional 1D4 epitope
Embodiment (L): JellyOP (palm) -mGluR6
Construction: Box Jellyfish opsin chimera with the C-
terminus of murine GRM6 added behind the palmitoylation
site (grey Cys) of JellyOP
cDNA- SEQ No. 33
atgggagcgaatataacagaaatattgtcaggctttttagcttgtgtagtatttctctctataagcttgaatatgatagttctaattacatttt
accgcttacgacataaattggcttttaaagatgctctcatggctagtatggcgttcagtgatgttgtacaagctatcgtaggatatcctttag
aggtattcactgtagtagacggaaaatggacattcggaatggaattatgtcaagttgcaggatttttcattactgcccttggtcaagtttcc
atcgctcatctcactgctctcgccttagatcgatacttcacagtgtgcagacctttcgtggcaactgcgattcacggatcaatgagaaatgc
aggtatggtcatatttgtttgctggttctacgcgtccttctgggcagtcctacccttagttggatggtcaaattatgacgtggagggtgacgg
tatgcgatgctccatcaactgggcagacgacagtcctaaaagctactcatacagggtttgtttattcgtattcatctacctgattcctgttctt
ttgatggtggctacttacgtgttggttcaaggagagatgaagaatatgcgaggtcgtgcagcacagttgtttggttcagaatccgaggctg
cactaaagaatatcaaggccgaaaagcgacacacaaggctggttttcgtcatgatcctttctttcatcgtagcttggaccccatataccttc
gtcgccatgtgggtttcctttttcacgaaacaacttgggccaatacctttatacgttgatactttggcagctatgcttgcaaagtcgtcggct
atgttcaaccccatcatttactgcttcctccacaagcaattcagaagagctgtattacgtggtgtttgtctgttccatccagagcagaacgt
gcagaagcggaagcgcagcctcaagaagacctccacgatggcggccccgcccaagagcgagaactcagaggacgccaagacagaga
ccagccaagtggcgcctgccaagagcaggatcaccagcgagggcgagtacatccccctggaccagatcgacatcaacgtgtaa
Peptide sequence- SEQ No. 34
MGANITEILSGFLACVVFLSISLNMIVLITFYRLRHKLAFKDALMASMAFSDVVQAIVGYPLEVFTVVDGKWTF
GMELCQVAGFFITALGQVSIAHLTALALDRYFTVCRPFVATAIHGSMRNAGMVIFVCWFYASFWAVLPLVG
WSNYDVEGDGMRCSINWADDSPKSYSYRVCLFVFIYLIPVLLMVATYVLVQGEMKNMRGRAAQLFGSESE

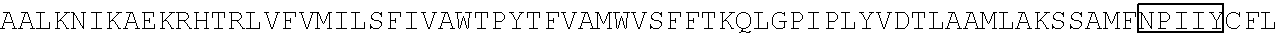

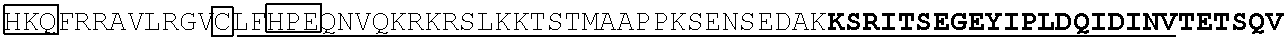

APA
Legend:
Underlined = GRM6
Underlined and bold = optional Golgi export signal
Bold = optional 1D4 epitope
Embodiment (M): JellyOP (palm) -5HT7
Construction: Box Jellyfish opsin chimera with the C-
terminus of the murine 5-hydroxytryptamine receptor 7
(isoform 1) added behind the palmitoylation site (grey
Cys) of JellyOP. The C-terminal sequence of the 5-HT7
receptor starting behind the palmitoylation site was
added here
cDNA- SEQ No. 35
atgggagcgaatataacagaaatattgtcaggctttttagcttgtgtagtatttctctctataagcttgaatatgatagttctaattacatttt
accgcttacgacataaattggcttttaaagatgctctcatggctagtatggcgttcagtgatgttgtacaagctatcgtaggatatcctttag
aggtattcactgtagtagacggaaaatggacattcggaatggaattatgtcaagttgcaggatttttcattactgcccttggtcaagtttcc
atcgctcatctcactgctctcgccttagatcgatacttcacagtgtgcagacctttcgtggcaactgcgattcacggatcaatgagaaatgc
aggtatggtcatatttgtttgctggttctacgcgtccttctgggcagtcctacccttagttggatggtcaaattatgacgtggagggtgacgg
tatgcgatgctccatcaactgggcagacgacagtcctaaaagctactcatacagggtttgtttattcgtattcatctacctgattcctgttctt
ttgatggtggctacttacgtgttggttcaaggagagatgaagaatatgcgaggtcgtgcagcacagttgtttggttcagaatccgaggctg
cactaaagaatatcaaggccgaaaagcgacacacaaggctggttttcgtcatgatcctttctttcatcgtagcttggaccccatataccttc
gtcgccatgtgggtttcctttttcacgaaacaacttgggccaatacctttatacgttgatactttggcagctatgcttgcaaagtcgtcggct
atgttcaaccccatcatttactgcttcctccacaagcaattcagaagagctgtattacgtggtgtttgtcagtaccggaatatcaaccggaa
gctctctgctgcaggcatgcacgaagccctgaaacttgctgagaggcctgagagaagcgagtttgtgctacaaaactgtgaccactgtgg
gaaaaaaggtcatgatacatgaaagagcaggatcaccagcgagggcgagtacatccccctggaccagatcgacatcaacgtgacag
agaccagccaagtggcgcctgcctaa
Peptide sequence- SEQ No. 36
MGANITEILSGFLACVVFLSISLNMIVLITFYRLRHKLAFKDALMASMAFSDVVQAIVGYPLEVFTVVDGKWTF
GMELCQVAGFFITALGQVSIAHLTALALDRYFTVCRPFVATAIHGSMRNAGMVIFVCWFYASFWAVLPLVG
WSNYDVEGDGMRCSINWADDSPKSYSYRVCLFVFIYLIPVLLMVATYVLVQGEMKNMRGRAAQLFGSESE

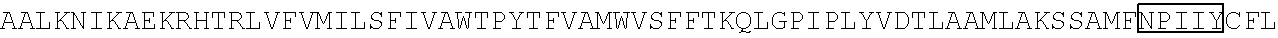

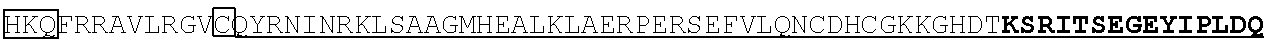

IDINVTETSQVAPA
Legend:
Underlined = 5HT7
Underlined and bold = optional Golgi export signal
Bold = optional 1D4 epitope
Embodiment (N): PPO (palm) -mGluR6 (murine mGluR6)
Construction: Lethenteron camtschaticum parapinopsin
(PPO)
Splicing site x at the palmitoylation siteof PPO fused
with the CT of mGluR6 at two amino acids upstream of the
HPE site.
DNA Sequence- SEQ No. 37
Ccatggagaacttgacctcgctcgacctcctgcccaacggcgaggtcccgttgatgccccgctacggcttcaccatcctcgccgtgatcat
ggccgtgttcaccatcgcctcgctcgtgctcaacagcaccgtcgtcatcgtcaccctgcgccaccgccagctacgccacccgctcaacttct
cgctcgtcaacctcgccgtggcggacctgggcgtcacggtgttcggcgccagcctcgtcgtggagaccaacgccgtcgggtacttcaacc
tcggccgcgtcggctgcgtcatcgaagggttcgccgtcgctttcttcggcatcgccgctctgtgcacgatcgccgtgatcgccgtcgatcgc
ttcgtggtggtgtgcaagccgctgggcacgctgatgttcacgcggcgccacgcgctgctgggcatcgcctgggcctggctctggtcgttcg
tgtggaacacgccgccgctcttcggctggggcagctacgagctggagggcgtgcggacgtcgtgcgcgcccgactggtacagccgcgac
cccgccaacgtgtcgtacattacgagctacttcgccttctgcttcgccatccccttcctcgtcatcgtggtggcgtacggccgcctcatgtgg
accctccaccaggtggccaagctggggatgggcgagagcggcagcaccgccaaggcggaggcgcaggtgtcgcgcatggtggtggtc
atggtggtggccttcctcgtctgctggctgccctacgcgctcttcgccatgatcgtggtgaccaagcccgacgtgtacatcgacccggtcat
cgccacactgcccatgtacctgaccaagacgagcacggtctacaaccccatcatctacatcttcatgaaccgccagttccgggactgcgc
cgtgcccttcctgctctgcctgttccatccagagcagaacgtgcagaagcggaagcgcagcctcaagaagacctccacgatggcggccc
cgcccaagagcgagaactcagaggacgccaagacagagaccagccaagtggcgcctgccaagagcaggatcaccagcgagggcga
gtacatccccctggaccagatcgacatcaacgtgtaa
Peptide sequence- SEQ No. 38
MENLTSLDLLPNGEVPLMPRYGFTILAVIMAVFTIASLVLNSTVVIVTLRHRQLRHPLNFSLVNLAVADL
GVTVFGASLVVETNAVGYFNLGRVGCVIEGFAVAFFGIAALCTIAVIAVDRFVVVCKPLGTLMFTRRHAL
LGIAWAWLWSFVWNTPPLFGWGSYELEGVRTSCAPDWYSRDPANVSYITSYFAFCFAIPFLVIVVAYGRL
MWTLHQVAKLGMGESGSTAKAEAQVSRMVVVMVVAFLVCWLPYALFAMIVVTKPDVYIDPVIATLPMYL

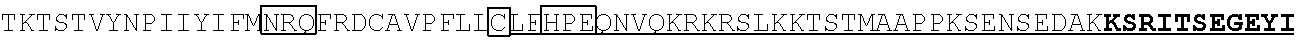

PLDQIDINVTETSQVAPA
Legend:
Underlined = GRM6
Underlined and bold = optional Golgi export signal
Bold = optional 1D4 epitope
Embodiment (O): JSR1 (palm) -mGluR6 (murine mGluR6)
Jumping spider rhodopsin, Kumpopsin 1 of Hasarious
adansoni
Construction: Splicing site x at the palmitoylation site
of JSR1 fused with the CT of mGluR6 at two amino acids
upstream of the HPE site.
cDNA Sequence- SEQ No. 39
atgttaccacatgcagcaaaaatggcggccagggtggctggcgatcacgatggaagaaacatttcaattgttgatcttctgccagaagac
atgctaccgatgattcacgaacattggtataagttccccccgatggaaacttccatgcattatatcctcggaatgcttattatagttatagg
aatcatcagtgtatcaggtaatggagttgtcatgtacttaatgatgacagtgaagaacctccgaactcccggtaattttctggtattgaatc
ttgccctatctgattttggtatgctgttttttatgatgccgacgatgtccataaattgcttcgccgaaacgtgggttataggacctttcatgtgt
gagctctatggaatgatcggctcattatttggaagtgcatctatctggagtctggtaatgataacactcgaccgatacaatgtcatcgtga
aaggaatggctggaaaacctctaacgaaagttggagcactgttaagaatgcttttcgtctggatttggtcattaggctggaccattgcacc
gatgtacggatggagcaggtatgttcctgaaggctcgatgacatcatgcactattgactacatagatacagctattaatccaatgtcttac
cttatcgcctacgctatttttgtatacttcgtaccactctttatcattatttactgctacgctttcatcgtaatgcaagtagcagcccacgaga
aatccctacgagaacaagctaaaaagatgaacatcaaatccctcagatcaaatgaagataacaagaaagcaagtgcagaattcagac
tggctaaggtggcttttatgaccatctgctgctggttcatggcgtggactccttatctaaccctgtccttccttggaatcttctccgacaggac
ctggctaacacctatgacatccgtttggggagccatatttgcaaaagctagtgcctgctacaatcctattgtttatggaataagtcatccta
agtatcgtgccgctttacatgataagttcccatgcctctgttccatccagagcagaacgtgcagaagcggaagcgcagcctcaagaagac
ctccacgatggcggccccgcccaagagcgagaactcagaggacgccaagacagagaccagccaagtggcgcctgccaagagcagga
tcaccagcgagggcgagtacatccccctggaccagatcgacatcaacgtgtaa
Peptide sequence- SEQ No. 40
MLPHAAKMAARVAGDHDGRNISIVDLLPEDMLPMIHEHWYKFPPMETSMHYILGMLIIVIGIISVSGNGV
VMYLMMTVKNLRTPGNFLVLNLALSDFGMLFFMMPTMSINCFAETWVIGPFMCELYGMIGSLFGSASIWS
LVMITLDRYNVIVKGMAGKPLTKVGALLRMLFVWIWSLGWTIAPMYGWSRYVPEGSMTSCTIDYIDTAIN
PMSYLIAYAIFVYFVPLFIIIYCYAFIVMQVAAHEKSLREQAKKMNIKSLRSNEDNKKASAEFRLAKVAFMTICC

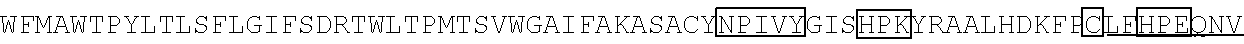

QKRKRSLKKTSTMAAPPKSENSEDAKKSRITSEGEYIPLDQIDINVTETSQVAPA
Legend:
Underlined = GRM6
Underlined and bold = optional Golgi export signal
Bold = optional 1D4 epitope

### REFERENCES

Ablonczy Z., Kono M., Knapp D. Crouch R., “Palmytolation of cone opsins” Vision Research 46 (2006) 4493-4501. doi: 10.1016/j.visres.2006.08.003
Ahn K H, Nishiyama A, Mierke D F, Kendall D A, “Hydrophobic residues in helix 8 of cannabinoid receptor 1 are critical for structural and functional properties.” Biochemistry (2010) 49: 502-11
Bailes, H. and R. Lucas, “Human melanopsin forms a pigment maximally sensitive to blue light (λmax≈479 nm) supporting activation of G(q/11) and G(i/o) signalling cascades. Proc Biol Sci, 2013. 280: p. 20122987. doi: 10.1098/rspb.2012.2987
Bruno A, Costantino G, De Fabritiis G, Pastor M, Selent J, “Membrane-Sensitive Conformational States of Helix 8 in Metabotropic Glu2 Receptor, a Class C GPCR”. PLoS ONE, vol. 7, Issue 8, August 2012. (https://doi.org/10.1371/journal.pone.0042023)
Cronin T., Vandenberghe L., Hantz P., Juttner J., Reimann A., Kacs6 A., Huckfeldt R., Busskamp V., Kohler H., Lagali P., Botond R.& Bennett J. “Efficient transduction and optogenetic stimulation of retinal bipolar cells by a synthetic adeno-associated virus capsid and promoter” EMBO Mol Med, 2014, 6(9): p. 1175-1190;
Cehajic-Kapetanovic J., Eleftheriou C., Allen A. E., Milosavljevic N., Pienaar A., Bedford R., Davis K. E., Bishop P. N., Lucas R. J., “Restoration of Vision with Ectopic Expression of Human Rod Opsin”. Curr Biol. 2015 Aug. 17; 25(16):2111-22. doi: 10.1016/j.cub.2015.07.029. Epub 2015 Jul. 30.
Dalkara D, Byrne L C, Klimczak R R, Visel M, Yin L, Merigan W H, Flannery J G, Schaffer D V, “In vivo-directed evolution of a new adeno-associated virus for therapeutic outer retinal gene delivery from the vitreous” Sci Transl Med. 2013 Jun. 12; 5(189):189ra76. doi: 10.1126/scitranslmed.3005708
David A., “Evolution of recombinant adeno-associated viral vectors for application in ocular gene therapy” Dissertation Universität Bern, 2018.
Davies W, Hankinsa M, Foster R; “Vertebrate ancient opsin and melanopsin: divergent irradiance detectors”. 2010, Photochem. Photobiol. Sci., 2010, 9, 1444-1457.
Fritze O, Slawomir F, Kuksa V, Palczewski K, Hofmann K, Ernst O, “Role of the conserved NPxxY(x)_{5,6}F (SEQ ID NO: 91) motif in the rhodopsin ground state and during activation”. PNAS, Mar. 4, 2003, vol. 100, No. 5. (https://www.ncbi.nlm.nih.gov/pmc/articles/PMC151333/)
Gross A. K. et al., Defective Membrane Trafficking of Autosomal Dominant Retinitis Pigmentosa Mutant Ter349Glu Rhodopsin I S Rescued by C-Terminal Addition of 1D4 Epitope, iovs, April 2009, Vol. 50, Issue 13
https://molbiol-tools.ca/Protein tertiary_structure.htm, accessed on 27 Nov. 2020, 10:40 am
https://www.schrodinger.com/prime, accessed on 27 Nov. 2020, 10:45 am
Hulliger Carlos Elmar et al., Empowering Retianl Gene Therapy with a Specific Promoter of Human Rod and Cone ON-Bipolar Cells, Molecular Therapy: Methods & Clinical Development, Vol. 17, June 2020. pp. 505-519
Hulliger Carlos Elmar et al., Empowering Retianl Gene Therapy with a Specific Promoter of Human Rod and Cone ON-Bipolar Cells, Molecular Therapy: Methods & Clinical Development, Vol. 17, June 2020. pp. 505-519—Supplemental Information
Hsu P., Lander E. S., Zhang F., “Development and Applications of CRISPR-Cas9 for Genome Engineering”, Cell 157(6):1262-1278 (2014)
Kim D, Matsuda T and Cepko C. “A Core Paired-Type and POU Homeodomain-Containing Transcription Factor Program Drives Retinal Bipolar Cell Gene Expression”, J Neurosci 2008, 28: p. 7748-7764;
Kleinlogel S, “Optogenetic user's guide to Opto-GPCRs”. Frontiers in Bioscience, Landmark, 21, 794-805, Jan. 1, 2016.
Kuhlman, B., Bradley, P., “Advances in protein structure prediction and design”. Nat Rev Mol Cell Biol 20, 681-697 (2019)
Lagali P. S., Balya D., Awatramani G. B., Minch T. A., Kim D. S., Busskamp V., Cepko C. L., Roska B, “Light-activated channels targeted to ON bipolar cells restore visual function in retinal degeneration”. Nat Neurosci. 2008 June; 11(6):667-75. doi: 10.1038/nn.2117. Epub 2008 Apr. 27.
Lin K., Simossis V. A., Taylor W. R., Heringa J., “A simple and fast secondary structure prediction method using hidden neural networks”, Bioinformatics 21(2):152-159 (2005)
Lodowski et al., Signals Governing the Trafficking and Mistrafficking of a Ciliary GPCR, Rhodopsin, The Journal of Neuroscience, Aug. 21, 2013, −33(34):13621-13638.13621
Long C., Li H., Tiburcy M., Rodriguez-Caycedo C., Kyrychenko V., Zhou H., Zhang Y., Min Y., Shelton J. M., Mammen P. A., Liaw N. Y., Zimmermann W. H., Bassel-Duby R., Schneider J. W. and Olson E. N., “Correction of diverse muscular dystrophy mutations in human engineered heart muscle by single-site genome editing”, Science Advances 4(1) (2018) eaap9004, DOI: 10.1126/sciadv.aap9004
Lu Q, Ganjawala T, Ivanova E, Cheng J, Troilo D and Pan Z, “AAV-mediated transduction and targeting of retinal bipolar cells with improved mGluR6 promoters in rodents and primates”, Gene Ther, 2016, 23: p. 680-9.
Moreira I S, “Structural features of the G-protein/GPCR interactions”. Biochimica et Bioohysica Acta Vol. 1840_1, p. 16-33, January 2014 (https://doi.org/10.1016/j.bbagen.2013.08.027)
Morri M, Sanchez-Romero I, Tichy A M, Kainrath S, Gerrard E J, Hirschfeld P P, Schwarz J, Janovjak H “Optical functionalization of human Class A orphan G-protein-coupled receptors.” Nat Commun 9: 1950 (2018), doi:10.1038/s41467-018-04342-1 (2018).
Mure L, Hatori M, Zhu Q, Demas J, Kim I, Nayak S, Panda S, “Melanopsin-Encoded Response Properties of Intrinsically Photosensitive Retinal Ganglion Cells”. 2016, Neuron 90, 1016-1027, Jun. 1, 2016; https://doi.org/10.1016/j.neuron.2016.04.016)
Nygaard R et al. “The Dynamic Process of β-Adrenergic Receptor activation”. Cell 152, 532-542, Jan. 31, 2013. (https://doi.org/10.1016/j.bbagen.2013.08.027)
O'Brien, J. A., Lummis, S. C., “Nano-biolistics: a method of biolistic transfection of cells and tissues using a gene gun with novel nanometer-sized projectiles”, BMC Biotechnol 11, 66 (2011). https://doi.org/10.1186/1472-6750-11-66
Ovchinnikov Yu A, Abdulaev N G, Bogachuk A S, “Two adjacent cysteine residues in the C-terminal cytoplasmic fragment of bovine rhodopsinhttps”, //doi.org/10.1016/j.bbagen.2013.08.027 are palmitilated. Federation of European Biochemical Societies Letters 1988; 230:1-5. [PubMed: 3350146]
Prusky G. T., Alam N. M., Beekman, S., Douglas R. M., “Rapid Quantification of Adult and Developing Mouse Spatial Vision Using a Virtual Optomotor System”, 45:4611-4616, December 2004
Santello M., Nevian T., “Dysfunction of Cortical Dendritic Integration in Neuropathic Pain Reversed by Serotoninergic Neuromodulation”, Neuron 86(1):233-246, Mar. 26, 2015
Sato T., “Conserved 2nd Residue of Helix 8 of GPCR May Confer the Subclass-Characteristic and Distinct Roles through a Rapid Initial Interaction with Specific G Proteins”, Int. J. Mol. Sci. 2019, 20, 1752; doi:10.3390/ijms20071752.
Schiöth, H. B, Fredriksson R, “The GRAFS classification system of G-protein coupled receptors in comparative perspective”. General and Comparative Endocrinology 142, Feb. 15, 2005, 94-101. Doi: 10.1016/j.ygcen.2004.12.018.
Schwartz T, Frimurer T, Holst B, Rosenkilde M and Elling C, “Molecular mechanism of 7TM receptor activation—a global toggle switch model”. Annu Rev Pharmacol Toxicol 46, 481-519 (2006—DOI: 10.1146/annurev.pharmtox.46.120604.141218.
Siuda E, Copits B, Schmidt M, Baird M, Al-Hasani R, Planer W, Funderburk S, McCall J, Gereau I V R, Bruchas M, “Spatiotemporal control of opioid signaling and behavior”. Neuron. 2015 May 20; 86(4): 923-935. doi:10.1016/j.neuron.2015.03.066
Somasundaram P, Wyrick G R, Fernandez D C, Ghahari A, Pinhal C M, Richardson M S, Rupp A C, Cui L, Wu Z, Brown R L, Badea T C, Hattar S, and Robinson P R., “C-terminal phosphorylation regulates the kinetics of a subset of melanopsin-mediated behaviors in mice”. Proc Natl Acad Sci USA. 2017 Mar. 7; 114(10): 2741-2746.
Sriram K and Insel P A, “G Protein-Coupled Receptors as Targets for Approved Drugs: How Many Targets and How Many Drugs?”. Mol Pharmacol 93:251-258, April 2018. https://doi.org/10.1124/mol.117.111062
Tsai C-J, Pamula F, Nehme R, Muhle J, Weinert T, Flock T, Nogly P, Edwards P, Carpenter B, Gruhl T, Ma P, Deupi X, Standfuss J, Tate C and Schertler G, “Crystal structure of rhodopsin in complex with a mini-Go sheds light on the principles of G protein selectivity”, Sci. Adv. 2018; 4: eaat 7052 19 Sep. 2018
Tsai C J, Marino J, Adaixo R, Pamula F, Muehle J, Maeda S, Flock T, Taylor N M, Mohammed I, Matile H, Dawson R J, Deupi X, Stahlberg H, Schertler G, “Cryo-E M structure of the rhodopsin-Gαi-βγ complex reveals binding of the rhodopsin C-terminal tail to the GB subunit”. Elife 2019 Jun. 28; 8: e46041. Doi: 10.7554/eLife.46041
Vandemoortele, G., De Sutter, D. and Eyckerman, S., “Robust Generation of Knock-in Cell Lines Using CRISPR-Cas9 and rAAV-assisted Repair Template Delivery”. Bio-protocol 7(7): e2211 (2017). DOI: 10.21769/BioProtoc.2211.
van Wyk M., Pielecka-Fortuna J., Lowel S., Kleinlogel S., “Restoring the ON Switch in Blind Retinas: Chimeric opsin mGluR6 proteins, a Next-Generation, Cell-Tailored Optogenetic Tool”. PLoS Biol. 2015 May 7; 13(5):e1002143. doi: 10.1371/journal.pbio.1002143. eCollection 2015 May.
Zhu X, Brown B, Li A, Mears A J, Swaroop A, Craft C M, “GRK1-dependent phosphorylation of S and M opsins and their binding to cone arrestin during cone phototransduction in the mouse retina.” J Neurosci. 2003 Jul. 9, 23(14):6152-60.

While there were shown and described above presently preferred embodiments of the invention, it is to be distinctly understood that the invention is not limited thereto but may be otherwise variously embodied and practiced within the scope of the following claims.

1-85. (canceled)

86. A chimeric opsin GPCR protein comprising seven transmembrane domains (TM1 to TM7) connected by extra- and intracellular loops (ELs and ILs),
wherein the chimeric opsin GPCR protein comprises a first light-sensitive opsin portion and a second GPCR portion (target GPCR portion);
wherein the first light-sensitive opsin portion comprises a chromophore pocket covalently bound to a chromophore and a truncated C-terminal (CT) domain with a truncation site located at least 7 amino acids downstream of a NR(K)Q motif,
and
wherein the second GPCR portion (target GPCR portion) comprises a C-terminal domain (target-GPCR-CT) positioned downstream of the truncated C-terminal domain of the opsin portion.

87. The chimeric opsin GPCR protein of claim 86, wherein the truncation site is positioned at or downstream of:
a distal end of a helix 8 (H8); or
a palmitoylation site.

88. The chimeric opsin GPCR protein of claim 87, wherein the truncation site is positioned up to 33 amino acids downstream of the palmitoylation site.

89. The chimeric opsin GPCR protein of claim 86, wherein the first light-sensitive opsin portion is derived from a melanopsin, rhodopsin, a cone opsin, a cone opsin selected from OPN1SW, OPN1LW and OPN1MW, a jellyfish opsin, a jellyfish opsin selected from cubop and JellyOP, jumping spider rhodopsin (JSR1), Parapinopsin (PPO), Neuropsin (OPN5), or Encephalopsin (OPN3).

90. The chimeric opsin GPCR protein of claim 86, wherein the first light-sensitive opsin portion is derived from two or more opsins.

91. The chimeric opsin GPCR protein of claim 86, wherein the first light-sensitive opsin portion comprises an entire opsin from the N-terminus up to the truncation site.

92. The chimeric opsin GPCR protein of claim 86, wherein the first light-sensitive opsin portion is derived from a mono-stable opsin and/or from a bi-stable opsin and/or from a tri-stable opsin.

93. The chimeric opsin GPCR protein of claim 86, wherein:
a) the target-GPCR-CT is a functional variant of a C-terminal (CT) domain of a GPCR from which the target-GPCR-CT is derived;
b) the target-GPCR-CT comprises an N terminal deletion between an NPxxY motif and any amino acid position up to a palmitoylation site; and/or
c) the N-terminal end of the target-GPCR-CT is at or upstream of the NR(K)Q motif.

94. The chimeric opsin GPCR protein according to claim 86, wherein the target GPCR portion is derived from a class C GPCR, or wherein the target GPCR portion is derived from mGluR6.

95. The chimeric opsin GPCR protein of claim 86, wherein the CT of the chimeric opsin GPCR further comprises:
a Golgi export signal;
a membrane trafficking sequence, or a membrane trafficking sequence which is a 1D4 tag; and/or
a sequence element encoding a fluorescent protein,
and wherein the one or more selected elements are arranged independently in any order at the C-terminal end of the chimeric opsin GPCR CT.

96. The chimeric opsin GPCR protein of claim 86, wherein the target-GPCR-CT is capable of coupling light activation into the signaling pathway of a GPCR from which the target-GPCR-CT is derived.

97. The chimeric opsin GPCR protein of claim 86, wherein the first light-sensitive opsin portion is derived from a melanopsin and the target GPCR portion is derived from mGluR6.

98. The chimeric opsin GPCR protein of claim 86, comprising an amino acid sequence with at least 85% identity to an amino acid sequence of SEQ ID NO 2, SEQ ID NO 4, SEQ ID NO 6, SEQ ID NO 8, SEQ ID NO 10 and SEQ ID NO 12, SEQ ID NO 14, SEQ ID NO 16, SEQ ID NO 18, SEQ ID NO 20, SEQ ID NO 22, SEQ ID NO 24, SEQ ID NO 26, SEQ ID NO 28, SEQ ID NO 30 and SEQ ID NO 32 SEQ ID NO 34, SEQ ID NO 36, SEQ ID NO 38, SEQ ID NO 40, SEQ ID NO 42 or SEQ ID NO 44.

99. The chimeric opsin GPCR protein according to claim 98 comprising an amino acid sequence of SEQ ID NO 2, SEQ ID NO 4, SEQ ID NO 6, SEQ ID NO 8, SEQ ID NO 10 and SEQ ID NO 12, SEQ ID NO 14, SEQ ID NO 16, SEQ ID NO 18, SEQ ID NO 20, SEQ ID NO 22, SEQ ID NO 24, SEQ ID NO 26, SEQ ID NO 28, SEQ ID NO 30 and SEQ ID NO 32 SEQ ID NO 34, SEQ ID NO 36, SEQ ID NO 38, SEQ ID NO 40, SEQ ID NO 42 or SEQ ID NO 44.

100. The chimeric opsin GPCR protein of claim 98, wherein the amino acid sequence comprises:
a conservative amino acid substitution;
a deletion in a range of 1 up to 3, 5, 8 or 15 amino acids; and/or
an insertion in a range of 1 up to 3, 5, 8 or 15 amino acids;
and wherein the chimeric opsin GPCR protein exhibits a light activation dependent binding of Galpha protein specific to GPCR from which the target-GPCR-CT is derived.

101. A nucleic acid molecule encoding the chimeric opsin GPCR protein of claim 86.

102. A vector comprising the nucleic acid molecule of claim 101, operably linked to a promoter.

103. The vector of claim 102, wherein the promoter is a bipolar cell specific promoter.

104. A vector comprising a transgene encoding the chimeric opsin GPCR protein of claim 86, operably linked to a promoter.

105. The vector according to claim 102, wherein the vector is a recombinant adeno-associated virus (rAAV) selected from an AAV1, AAV2, AAV3, AAV4, AAV5, AAV6, AAV7, AAV8, AAV9, AAV10, AAV 11 and AAV12 serotype.

106. A method of treating partial or complete blindness, retinitis pigmentosa (RP), macular degeneration, or other forms of photoreceptor degeneration, comprising administering the chimeric opsin GPCR protein of claim 86 to a patient in need thereof.

107. A method of treating partial or complete blindness, retinitis pigmentosa (RP), macular degeneration, or other forms of photoreceptor degeneration, comprising administering the nucleic acid of claim 101 to a patient in need thereof.

108. A method of treating partial or complete blindness, retinitis pigmentosa (RP), macular degeneration, or other forms of photoreceptor degeneration, comprising administering the vector of claim 102 to a patient in need thereof.


SEQUENCE LISTING (99 sequences)

SEQ ID NO: 1 - DNA, 1377 bp, Artificial Sequence
  Description of Artificial Sequence Mela(palm+33AA)-mGluR6 cDNA
atggactctccttcaggaccaagagtcttgtcaagcttaactcaggatcccagcttcacaaccagtcctgccctgcaaggcatttggaacggcactcagaacgtctccgtaagagcccagcttctctctgttagccccacgacatctgcacatcaggctgctgcctgggtccccttccccacagtcgatgtcccagaccatgctcactataccctaggcacggtgatcctgctggtgggactcacagggatgctgggcaatctgacggtcatctacaccttctgcaggaacagaggcctgcggacaccagcaaacatgttcatcatcaacctcgcagtcagcgacttcctcatgtcagtcactcaggccccggtcttctttgccagcagcctctacaagaagtggctctttggggagacaggttgcgagttctatgccttctgcggggctgtctttggcatcacttccatgatcaccctgacagccatagccatggaccgctatctggtgatcacacgtccactggccaccatcggcaggggatccaaaagacgaacggcactcgtcctgctaggcgtctggctttatgccctggcctggagtctgccacctttctttggttggagtgcctacgtgcccgaggggctgctgacatcctgctcctgggactacatgaccttcacaccccaggtgcgtgcctacaccatgctgctcttctgctttgtcttcttcctccccctgctcatcatcatcttctgctacatcttcatcttcagggccatccgagagacaggccgggcctgtgagggctgcggtgagtcccctctgcggcagaggcggcagtggcagcggctgcagagtgagtggaagatggccaaggtcgcactgattgtcattcttctcttcgtgctgtcctgggctccctactccactgtggctctggtggcctttgctggatactcgcacatcctgacgccctacatgagctcggtgccagccgtcatcgccaaggcttctgccatccacaatcccattatctacgccatcactcaccccaagtacagggtggccattgcccagcacctgccttgccttggggtgcttctcggtgtatcaggccagcgcagccacccctccctcagctaccgctctacccaccgctccacattgagcagccagtcctcagacctccatccagagcagaacgtgcagaagcggaagcgcagcctcaagaagacctccacgatggcggccccgcccaagagcgagaactcagaggacgccaagaagagcaggatcaccagcgagggcgagtacatccccctggaccagatcgacatcaacgtgacagagaccagccaagtggcgcctgcctaa

SEQ ID NO: 2 - protein, 458 aa, Artificial Sequence
  Description of Artificial Sequence Mela(palm+33AA)-mGluR6 protein
  LIPID (364)...(364): PALMITATE
MDSPSGPRVLSSLTQDPSFTTSPALQGIWNGTQNVSVRAQLLSVSPTTSAHQAAAWVPFPTVDVPDHAHYTLGTVILLVGLTGMLGNLTVIYTFCRNRGLRTPANMFIINLAVSDFLMSVTQAPVFFASSLYKKWLFGETGCEFYAFCGAVFGITSMITLTAIAMDRYLVITRPLATIGRGSKRRTALVLLGVWLYALAWSLPPFFGWSAYVPEGLLTSCSWDYMTFTPQVRAYTMLLFCFVFFLPLLIIIFCYIFIFRAIRETGRACEGCGESPLRQRRQWQRLQSEWKMAKVALIVILLFVLSWAPYSTVALVAFAGYSHILTPYMSSVPAVIAKASAIHNPIIYAITHPKYRVAIAQHLPCLGVLLGVSGQRSHPSLSYRSTHRSTLSSQSSDLHPEQNVQKRKRSLKKTSTMAAPPKSENSEDAKKSRITSEGEYIPLDQIDINVTETSQVAPA

SEQ ID NO: 3 - DNA, 1284 bp, Artificial Sequence
  Description of Artificial Sequence Mela(palm)-mGluR6 cDNA
atggactctccttcaggaccaagagtcttgtcaagcttaactcaggatcccagcttcacaaccagtcctgccctgcaaggcatttggaacggcactcagaacgtctccgtaagagcccagcttctctctgttagccccacgacatctgcacatcaggctgctgcctgggtccccttccccacagtcgatgtcccagaccatgctcactataccctaggcacggtgatcctgctggtgggactcacagggatgctgggcaatctgacggtcatctacaccttctgcaggaacagaggcctgcggacaccagcaaacatgttcatcatcaacctcgcagtcagcgacttcctcatgtcagtcactcaggccccggtcttctttgccagcagcctctacaagaagtggctctttggggagacaggttgcgagttctatgccttctgcggggctgtctttggcatcacttccatgatcaccctgacagccatagccatggaccgctatctggtgatcacacgtccactggccaccatcggcaggggatccaaaagacgaacggcactcgtcctgctaggcgtctggctttatgccctggcctggagtctgccacctttctttggttggagtgcctacgtgcccgaggggctgctgacatcctgctcctgggactacatgaccttcacaccccaggtgcgtgcctacaccatgctgctcttctgctttgtcttcttcctccccctgctcatcatcatcttctgctacatcttcatcttcagggccatccgagagacaggccgggcctgtgagggctgcggtgagtcccctctgcggcagaggcggcagtggcagcggctgcagagtgagtggaagatggccaaggtcgcactgattgtcattcttctcttcgtgctgtcctgggctccctactccactgtggctctggtggcctttgctggatactcgcacatcctgacgccctacatgagctcggtgccagccgtcatcgccaaggcttctgccatccacaatcccattatctacgccatcactcaccccaagtacagggtggccattgcccagcacctgccttgcctgttccatccagagcagaacgtgcagaagcggaagcgcagcctcaagaagacctccacgatggcggccccgcccaagagcgagaactcagaggacgccaagaagagcaggatcaccagcgagggcgagtacatccccctggaccagatcgacatcaacgtgacagagaccagccaagtggcgcctgcctaa

SEQ ID NO: 4 - protein, 427 aa, Artificial Sequence
  Description of Artificial Sequence Mela(palm)-mGluR6 protein
  LIPID (364)...(364): PALMITATE
MDSPSGPRVLSSLTQDPSFTTSPALQGIWNGTQNVSVRAQLLSVSPTTSAHQAAAWVPFPTVDVPDHAHYTLGTVILLVGLTGMLGNLTVIYTFCRNRGLRTPANMFIINLAVSDFLMSVTQAPVFFASSLYKKWLFGETGCEFYAFCGAVFGITSMITLTAIAMDRYLVITRPLATIGRGSKRRTALVLLGVWLYALAWSLPPFFGWSAYVPEGLLTSCSWDYMTFTPQVRAYTMLLFCFVFFLPLLIIIFCYIFIFRAIRETGRACEGCGESPLRQRRQWQRLQSEWKMAKVALIVILLFVLSWAPYSTVALVAFAGYSHILTPYMSSVPAVIAKASAIHNPIIYAITHPKYRVAIAQHLPCLFHPEQNVQKRKRSLKKTSTMAAPPKSENSEDAKKSRITSEGEYIPLDQIDINVTETSQVAPA

SEQ ID NO: 5 - DNA, 1389 bp, Artificial Sequence
  Description of Artificial Sequence Mela(palm+33AA) -mGluR6(IL1, CT) cDNA
atggactctccttcaggaccaagagtcttgtcaagcttaactcaggatcccagcttcacaaccagtcctgccctgcaaggcatttggaacggcactcagaacgtctccgtaagagcccagcttctctctgttagccccacgacatctgcacatcaggctgctgcctgggtccccttccccacagtcgatgtcccagaccatgctcactataccctaggcacggtgatcctgctggtgggactcacagggatgctgggcaatctgacggtcatctacaccttcatgcgacacaacgacactcccatagtccgcgcctctggccgtgagcttttcatcatcaacctcgcagtcagcgacttcctcatgtcagtcactcaggccccggtcttctttgccagcagcctctacaagaagtggctctttggggagacaggttgcgagttctatgccttctgcggggctgtctttggcatcacttccatgatcaccctgacagccatagccatggaccgctatctggtgatcacacgtccactggccaccatcggcaggggatccaaaagacgaacggcactcgtcctgctaggcgtctggctttatgccctggcctggagtctgccacctttctttggttggagtgcctacgtgcccgaggggctgctgacatcctgctcctgggactacatgaccttcacaccccaggtgcgtgcctacaccatgctgctcttctgctttgtcttcttcctccccctgctcatcatcatcttctgctacatcttcatcttcagggccatccgagagacaggccgggcctgtgagggctgcggtgagtcccctctgcggcagaggcggcagtggcagcggctgcagagtgagtggaagatggccaaggtcgcactgattgtcattcttctcttcgtgctgtcctgggctccctactccactgtggctctggtggcctttgctggatactcgcacatcctgacgccctacatgagctcggtgccagccgtcatcgccaaggcttctgccatccacaatcccattatctacgccatcactcaccccaagtacagggtggccattgcccagcacctgccttgccttggggtgcttctcggtgtatcaggccagcgcagccacccctccctcagctaccgctctacccaccgctccacattgagcagccagtcctcagacctccatccagagcagaacgtgcagaagcggaagcgcagcctcaagaagacctccacgatggcggccccgcccaagagcgagaactcagaggacgccaagaagagcaggatcaccagcgagggcgagtacatccccctggaccagatcgacatcaacgtgacagagaccagccaagtggcgcctgcctaa

SEQ ID NO: 6 - protein, 462 aa, Artificial Sequence
  Description of Artificial Sequence Mela(palm+33AA) -mGluR6(IL1, CT) protein
  LIPID (368)...(368): PALMITATE
MDSPSGPRVLSSLTQDPSFTTSPALQGIWNGTQNVSVRAQLLSVSPTTSAHQAAAWVPFPTVDVPDHAHYTLGTVILLVGLTGMLGNLTVIYTFMRHNDTPIVRASGRELFIINLAVSDFLMSVTQAPVFFASSLYKKWLFGETGCEFYAFCGAVFGITSMITLTAIAMDRYLVITRPLATIGRGSKRRTALVLLGVWLYALAWSLPPFFGWSAYVPEGLLTSCSWDYMTFTPQVRAYTMLLFCFVFFLPLLIIIFCYIFIFRAIRETGRACEGCGESPLRQRRQWQRLQSEWKMAKVALIVILLFVLSWAPYSTVALVAFAGYSHILTPYMSSVPAVIAKASAIHNPIIYAITHPKYRVAIAQHLPCLGVLLGVSGQRSHPSLSYRSTHRSTLSSQSSDLHPEQNVQKRKRSLKKTSTMAAPPKSENSEDAKKSRITSEGEYIPLDQIDINVTETSQVAPA

SEQ ID NO: 7 - DNA, 1284 bp, Artificial Sequence; identical to SEQ ID NO: 3
  Description of Artificial Sequence Mela(palm)-mGluR6(IL1, CT) cDNA

SEQ ID NO: 8 - protein, 431 aa, Artificial Sequence
  Description of Artificial Sequence Mela(palm)-mGluR6(IL1, CT) protein
  LIPID (368)...(368): PALMITATE
MDSPSGPRVLSSLTQDPSFTTSPALQGIWNGTQNVSVRAQLLSVSPTTSAHQAAAWVPFPTVDVPDHAHYTLGTVILLVGLTGMLGNLTVIYTFMRHNDTPIVRASGRELFIINLAVSDFLMSVTQAPVFFASSLYKKWLFGETGCEFYAFCGAVFGITSMITLTAIAMDRYLVITRPLATIGRGSKRRTALVLLGVWLYALAWSLPPFFGWSAYVPEGLLTSCSWDYMTFTPQVRAYTMLLFCFVFFLPLLIIIFCYIFIFRAIRETGRACEGCGESPLRQRRQWQRLQSEWKMAKVALIVILLFVLSWAPYSTVALVAFAGYSHILTPYMSSVPAVIAKASAIHNPIIYAITHPKYRVAIAQHLPCLFHPEQNVQKRKRSLKKTSTMAAPPKSENSEDAKKSRITSEGEYIPLDQIDINVTETSQVAPA

SEQ ID NO: 9 - DNA, 1380 bp, Artificial Sequence
  Description of Artificial Sequence Mela(palm+33AA)-mGluR6(IL3, CT) cDNA
atggactctccttcaggaccaagagtcttgtcaagcttaactcaggatcccagcttcacaaccagtcctgccctgcaaggcatttggaacggcactcagaacgtctccgtaagagcccagcttctctctgttagccccacgacatctgcacatcaggctgctgcctgggtccccttccccacagtcgatgtcccagaccatgctcactataccctaggcacggtgatcctgctggtgggactcacagggatgctgggcaatctgacggtcatctacaccttctgcaggaacagaggcctgcggacaccagcaaacatgttcatcatcaacctcgcagtcagcgacttcctcatgtcagtcactcaggccccggtcttctttgccagcagcctctacaagaagtggctctttggggagacaggttgcgagttctatgccttctgcggggctgtctttggcatcacttccatgatcaccctgacagccatagccatggaccgctatctggtgatcacacgtccactggccaccatcggcaggggatccaaaagacgaacggcactcgtcctgctaggcgtctggctttatgccctggcctggagtctgccacctttctttggttggagtgcctacgtgcccgaggggctgctgacatcctgctcctgggactacatgaccttcacaccccaggtgcgtgcctacaccatgctgctcttctgctttgtcttcttcctccccctgctcatcatcatcttctgctacatcttcatcttcagggccatccgagagacaggccggggtgtgccagagaccttcaatgaagcccctctgcggcagaggcggcagtggcagcggctgcagagtgagtggaagatggccaaggtcgcactgattgtcattcttctcttcgtgctgtcctgggctccctactccactgtggctctggtggcctttgctggatactcgcacatcctgacgccctacatgagctcggtgccagccgtcatcgccaaggcttctgccatccacaatcccattatctacgccatcactcaccccaagtacagggtggccattgcccagcacctgccttgccttggggtgcttctcggtgtatcaggccagcgcagccacccctccctcagctaccgctctacccaccgctccacattgagcagccagtcctcagacctccatccagagcagaacgtgcagaagcggaagcgcagcctcaagaagacctccacgatggcggccccgcccaagagcgagaactcagaggacgccaagaagagcaggatcaccagcgagggcgagtacatccccctggaccagatcgacatcaacgtgacagagaccagccaagtggcgcctgcctaa

SEQ ID NO: 10 - protein, 459 aa, Artificial Sequence
  Description of Artificial Sequence Mela(palm+33AA)-mGluR6(IL3, CT) protein
  LIPID (365)...(365): PALMITATE
MDSPSGPRVLSSLTQDPSFTTSPALQGIWNGTQNVSVRAQLLSVSPTTSAHQAAAWVPFPTVDVPDHAHYTLGTVILLVGLTGMLGNLTVIYTFCRNRGLRTPANMFIINLAVSDFLMSVTQAPVFFASSLYKKWLFGETGCEFYAFCGAVFGITSMITLTAIAMDRYLVITRPLATIGRGSKRRTALVLLGVWLYALAWSLPPFFGWSAYVPEGLLTSCSWDYMTFTPQVRAYTMLLFCFVFFLPLLIIIFCYIFIFRAIRETGRGVPETFNEAPLRQRRQWQRLQSEWKMAKVALIVILLFVLSWAPYSTVALVAFAGYSHILTPYMSSVPAVIAKASAIHNPIIYAITHPKYRVAIAQHLPCLGVLLGVSGQRSHPSLSYRSTHRSTLSSQSSDLHPEQNVQKRKRSLKKTSTMAAPPKSENSEDAKKSRITSEGEYIPLDQIDINVTETSQVAPA

SEQ ID NO: 11 - DNA, 1164 bp, Artificial Sequence
  Description of Artificial Sequence Mela(palm)-OPN1MW(IL1, IL2, IL3, CT) cDNA
atggactctccttcaggaccaagagtcttgtcaagcttaactcaggatcccagcttcacaaccagtcctgccctgcaaggcatttggaacggcactcagaacgtctccgtaagagcccagcttctctctgttagccccacgacatctgcacatcaggctgctgcctgggtccccttccccacagtcgatgtcccagaccatgctcactataccctaggcacggtgatcctgctggtgggactcacagggatgctgggcaatctgacggtcatctacgccaccatgagattcaagaagctgcgccatccagcaaacatgttcatcatcaacctcgcagtcagcgacttcctcatgtcagtcactcaggccccggtcttctttgccagcagcctctacaagaagtggctctttggggagacaggttgcgagttctatgccttctgcggggctgtctttggcatcacttccatgatcaccctgacagccatagccatggaccgctatctggtgatctgcaagccctttggcaatgtgagatttgatgctaagctgacggcactcgtcctgctaggcgtctggctttatgccctggcctggagtctgccacctttctttggttggagtgcctacgtgcccgaggggctgctgacatcctgctcctgggactacatgaccttcacaccccaggtgcgtgcctacaccatgctgctcttctgctttgtcttcttcctccccctgctcatcatcatcttctgctacatccaagtgtggctggccatccgagcagtggcaaagcaacagaaagaatctgagtccactcagaaggccgagaaggaggtgacaaaggtcgcactgattgtcattcttctcttcgtgctgtcctgggctccctactccactgtggctctggtggcctttgctggatactcgcacatcctgacgccctacatgagctcggtgccagccgtcatcgccaaggcttctgccatccacaatcccattatctacgccatcactcaccccaagtacagggtggccattgcccagcacctgccttgcaactgcatcttacatctctttggaaagaaggttgatgatagctctgaactttccagcacctccaagacagaagtctcatctgtctcttcagtgtcacctgcataa

SEQ ID NO: 12 - protein, 387 aa, Artificial Sequence
  Description of Artificial Sequence Mela(palm)-OPN1MW(IL1, IL2, IL3, CT) protein
  LIPID (353)...(353): PALMITATE
MDSPSGPRVLSSLTQDPSFTTSPALQGIWNGTQNVSVRAQLLSVSPTTSAHQAAAWVPFPTVDVPDHAHYTLGTVILLVGLTGMLGNLTVIYATMRFKKLRHPANMFIINLAVSDFLMSVTQAPVFFASSLYKKWLFGETGCEFYAFCGAVFGITSMITLTAIAMDRYLVICKPFGNVRFDAKLTALVLLGVWLYALAWSLPPFFGWSAYVPEGLLTSCSWDYMTFTPQVRAYTMLLFCFVFFLPLLIIIFCYIQVWLAIRAVAKQQKESESTQKAEKEVTKVALIVILLFVLSWAPYSTVALVAFAGYSHILTPYMSSVPAVIAKASAIHNPIIYAITHPKYRVAIAQHLPCNCILHLFGKKVDDSSELSSTSKTEVSSVSSVSPA

SEQ ID NO: 13 - DNA, 1092 bp, Artificial Sequence
  Description of Artificial Sequence OPN1MW(palm)-mGluR6 cDNA
atggcccaaaggcttacaggtgaacagacactggaccactatgaggatagcacccatgcaagcatcttcacctataccaacagcaacagcaccaaaggtccctttgaaggccccaattatcacattgctcccaggtgggtgtaccacctcaccagcacctggatgattcttgtggtcgttgcatctgtcttcactaatggacttgtgctggcagccaccatgagattcaagaagctgcgccatccactgaactggattctggtgaacttggcagttgctgacctagcagagaccattattgccagcactatcagtgttgtgaaccaaatctatggctacttcgttctgggacaccctctgtgtgtcattgaaggctacattgtctcattgtgtggaatcacaggcctctggtccctggccatcatttcctgggagagatggctggtggtctgcaagccctttggcaatgtgagatttgatgctaagctggccactgtgggaatcgtcttctcctgggtctgggctgctatatggacggccccaccaatctttggttggagcaggtactggccttatggcctgaagacatcctgtggcccagacgtgttcagcggtacctcgtaccccggggttcagtcttatatgatggtcctcatggtcacgtgctgcatcttcccactcagcatcatcgtgctctgctacctccaagtgtggctggccatccgagcagtggcaaagcaacagaaagaatctgagtccactcagaaggccgagaaggaggtgacacgcatggtggtggtgatggtcttcgcatactgcctctgctggggaccctatactttctttgcatgctttgctactgcccaccctggctatgccttccaccctcttgtggcctccctaccatcctactttgccaaaagtgccactatctacaaccccattatctatgtctttatgaaccggcagtttcgaaactgcatcttacatctctttcatccagagcagaacgtgcagaagcggaagcgcagcctcaagaagacctccacgatggcggccccgcccaagagcgagaactcagaggacgccaag

SEQ ID NO: 14 - protein, 393 aa, Artificial Sequence
  Description of Artificial Sequence OPN1MW(palm)-mGluR6 protein
MAQRLTGEQTLDHYEDSTHASIFTYTNSNSTKGPFEGPNYHIAPRWVYHLTSTWMILVVVASVFTNGLVLAATMRFKKLRHPLNWILVNLAVADLAETIIASTISVVNQIYGYFVLGHPLCVIEGYIVSLCGITGLWSLAIISWERWLVVCKPFGNVRFDAKLATVGIVFSWVWAAIWTAPPIFGWSRYWPYGLKTSCGPDVFSGTSYPGVQSYMMVLMVTCCIFPLSIIVLCYLQVWLAIRAVAKQQKESESTQKAEKEVTRMVVVMVFAYCLCWGPYTFFACFATAHPGYAFHPLVASLPSYFAKSATIYNPIIYVFMNRQFRNCILHLFHPEQNVQKRKRSLKKTSTMAAPPKSENSEDAKKSRITSEGEYIPLDQIDINVTETSQVAPA

SEQ ID NO: 15 - DNA, 1386 bp, Artificial Sequence
  Description of Artificial Sequence Mela(palm+33AA)-mGluR6 cDNA
atgaaccctccttcggggccaagagtcctgcccagcccaacccaagagcccagctgcatggccaccccagcaccacccagctggtgggacagctcccagagcagcatctccagcctgggccggcttccatccatcagtcccacagcacctgggacttgggctgctgcctgggtccccctccccacggttgatgttccagaccatgcccactataccctgggcacagtgatcttgctggtgggactcacggggatgctgggcaacctgacggtcatctataccttctgcaggagcagaagcctccggacacctgccaacatgttcattatcaacctcgcggtcagcgacttcctcatgtccttcacccaggcccctgtcttcttcaccagtagcctctataagcagtggctctttggggagacaggctgcgagttctatgccttctgtggagctctctttggcatttcctccatgatcaccctgacggccatcgccctggaccgctacctggtaatcacacgcccgctggccacctttggtgtggcgtccaagaggcgtgcggcatttgtcctgctgggcgtttggctctatgccctggcctggagtctgccacccttcttcggctggagcgcctacgtgcccgaggggttgctgacatcctgctcctgggactacatgagcttcacgccggccgtgcgtgcctacaccatgcttctctgctgcttcgtgttcttcctccctctgcttatcatcatctactgctacatcttcatcttcagggccatccgggagacaggacgggctctccagaccttcggggcctgcaagggcaatggcgagtccctgtggcagcggcagcggctgcagagcgagtgcaagatggccaagatcatgctgctggtcatcctcctcttcgtgctctcctgggctccctattccgctgtggccctggtggcctttgctgggtacgcacacgtcctgacaccctacatgagctcggtgccagccgtcatcgccaaggcctctgcaatccacaaccccatcatttacgccatcacccaccccaagtacagggtggccattgcccagcacctgccctgcctgggggtgctgctgggtgtatcacgccggcacagtcgcccctaccccagctaccgctccacccaccgctccacgctgaccagccacacctccaacctccatccagagcagaatgtgcagaagcgaaagcggagcctcaaggccacctccacggtggcagccccacccaagggcgaggatgcagaggcccacaagaagagcaggatcaccagcgagggcgagtacatccccctggaccagatcgacatcaacgtgacagagaccagccaagtggcgcctgcctaa

SEQ ID NO: 16 - protein, 461 aa, Artificial Sequence
  Description of Artificial Sequence Mela(palm+33AA)-mGluR6 protein
  LIPID (367)...(367): PALMITATE
MNPPSGPRVLPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLHPEQNVQKRKRSLKATSTVAAPPKGEDAEAHKKSRITSEGEYIPLDQIDINVTETSQVAPA

SEQ ID NO: 17 - DNA, 1293 bp, Artificial Sequence
  Description of Artificial Sequence Mela(palm)-mGluR6 L isoform of h melanopsin comprising a Golgi export signal and a 1D4 tag cDNA
atgaaccctccttcggggccaagagtcctgcccagcccaacccaagagcccagctgcatggccaccccagcaccacccagctggtgggacagctcccagagcagcatctccagcctgggccggcttccatccatcagtcccacagcacctgggacttgggctgctgcctgggtccccctccccacggttgatgttccagaccatgcccactataccctgggcacagtgatcttgctggtgggactcacggggatgctgggcaacctgacggtcatctataccttctgcaggagcagaagcctccggacacctgccaacatgttcattatcaacctcgcggtcagcgacttcctcatgtccttcacccaggcccctgtcttcttcaccagtagcctctataagcagtggctctttggggagacaggctgcgagttctatgccttctgtggagctctctttggcatttcctccatgatcaccctgacggccatcgccctggaccgctacctggtaatcacacgcccgctggccacctttggtgtggcgtccaagaggcgtgcggcatttgtcctgctgggcgtttggctctatgccctggcctggagtctgccacccttcttcggctggagcgcctacgtgcccgaggggttgctgacatcctgctcctgggactacatgagcttcacgccggccgtgcgtgcctacaccatgcttctctgctgcttcgtgttcttcctccctctgcttatcatcatctactgctacatcttcatcttcagggccatccgggagacaggacgggctctccagaccttcggggcctgcaagggcaatggcgagtccctgtggcagcggcagcggctgcagagcgagtgcaagatggccaagatcatgctgctggtcatcctcctcttcgtgctctcctgggctccctattccgctgtggccctggtggcctttgctgggtacgcacacgtcctgacaccctacatgagctcggtgccagccgtcatcgccaaggcctctgcaatccacaaccccatcatttacgccatcacccaccccaagtacagggtggccattgcccagcacctgccctgcctgttccatccagagcagaatgtgcagaagcgaaagcggagcctcaaggccacctccacggtggcagccccacccaagggcgaggatgcagaggcccacaagaagagcaggatcaccagcgagggcgagtacatccccctggaccagatcgacatcaacgtgacagagaccagccaagtggcgcctgcctaa

SEQ ID NO: 18 - protein, 430 aa, Artificial Sequence
  Description of Artificial Sequence Mela(palm)-mGluR6 L isoform of h melanopsin comprising a Golgi export signal and a 1D4 tag protein
  LIPID (367)...(367): PALMITATE
MNPPSGPRVLPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLFHPEQNVQKRKRSLKATSTVAAPPKGEDAEAHKKSRITSEGEYIPLDQIDINVTETSQVAPA

SEQ ID NO: 19 - DNA, 1293 bp, Artificial Sequence
  Description of Artificial Sequence Mela(palm)-mGluR6 P isoform of h melanopsin comprising a Golgi export signal and a 1D4 tag cDNA
atgaaccctccttcggggccaagagtcccgcccagcccaacccaagagcccagctgcatggccaccccagcaccacccagctggtgggacagctcccagagcagcatctccagcctgggccggcttccatccatcagtcccacagcacctgggacttgggctgctgcctgggtccccctccccacggttgatgttccagaccatgcccactataccctgggcacagtgatcttgctggtgggactcacggggatgctgggcaacctgacggtcatctataccttctgcaggagcagaagcctccggacacctgccaacatgttcattatcaacctcgcggtcagcgacttcctcatgtccttcacccaggcccctgtcttcttcaccagtagcctctataagcagtggctctttggggagacaggctgcgagttctatgccttctgtggagctctctttggcatttcctccatgatcaccctgacggccatcgccctggaccgctacctggtaatcacacgcccgctggccacctttggtgtggcgtccaagaggcgtgcggcatttgtcctgctgggcgtttggctctatgccctggcctggagtctgccacccttcttcggctggagcgcctacgtgcccgaggggttgctgacatcctgctcctgggactacatgagcttcacgccggccgtgcgtgcctacaccatgcttctctgctgcttcgtgttcttcctccctctgcttatcatcatctactgctacatcttcatcttcagggccatccgggagacaggacgggctctccagaccttcggggcctgcaagggcaatggcgagtccctgtggcagcggcagcggctgcagagcgagtgcaagatggccaagatcatgctgctggtcatcctcctcttcgtgctctcctgggctccctattccgctgtggccctggtggcctttgctgggtacgcacacgtcctgacaccctacatgagctcggtgccagccgtcatcgccaaggcctctgcaatccacaaccccatcatttacgccatcacccaccccaagtacagggtggccattgcccagcacctgccctgcctgttccatccagagcagaatgtgcagaagcgaaagcggagcctcaaggccacctccacggtggcagccccacccaagggcgaggatgcagaggcccacaagaagagcaggatcaccagcgagggcgagtacatccccctggaccagatcgacatcaacgtgacagagaccagccaagtggcgcctgcctaa

SEQ ID NO: 20 - protein, 430 aa, Artificial Sequence
  Description of Artificial Sequence Mela(palm)-mGluR6 P isoform of h melanopsin comprising a Golgi export signal and a 1D4 tag cDNAprotein
  LIPID (367)...(367): PALMITATE
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLFHPEQNVQKRKRSLKATSTVAAPPKGEDAEAHKKSRITSEGEYIPLDQIDINVTETSQVAPA

SEQ ID NO: 21 - DNA, 1266 bp, Artificial Sequence
  Description of Artificial Sequence Mela(palm)-mGluR6 L isoform of h melanopsin comprising a Golgi export signal cDNA
atgaaccctccttcggggccaagagtcctgcccagcccaacccaagagcccagctgcatggccaccccagcaccacccagctggtgggacagctcccagagcagcatctccagcctgggccggcttccatccatcagtcccacagcacctgggacttgggctgctgcctgggtccccctccccacggttgatgttccagaccatgcccactataccctgggcacagtgatcttgctggtgggactcacggggatgctgggcaacctgacggtcatctataccttctgcaggagcagaagcctccggacacctgccaacatgttcattatcaacctcgcggtcagcgacttcctcatgtccttcacccaggcccctgtcttcttcaccagtagcctctataagcagtggctctttggggagacaggctgcgagttctatgccttctgtggagctctctttggcatttcctccatgatcaccctgacggccatcgccctggaccgctacctggtaatcacacgcccgctggccacctttggtgtggcgtccaagaggcgtgcggcatttgtcctgctgggcgtttggctctatgccctggcctggagtctgccacccttcttcggctggagcgcctacgtgcccgaggggttgctgacatcctgctcctgggactacatgagcttcacgccggccgtgcgtgcctacaccatgcttctctgctgcttcgtgttcttcctccctctgcttatcatcatctactgctacatcttcatcttcagggccatccgggagacaggacgggctctccagaccttcggggcctgcaagggcaatggcgagtccctgtggcagcggcagcggctgcagagcgagtgcaagatggccaagatcatgctgctggtcatcctcctcttcgtgctctcctgggctccctattccgctgtggccctggtggcctttgctgggtacgcacacgtcctgacaccctacatgagctcggtgccagccgtcatcgccaaggcctctgcaatccacaaccccatcatttacgccatcacccaccccaagtacagggtggccattgcccagcacctgccctgcctgttccatccagagcagaatgtgcagaagcgaaagcggagcctcaaggccacctccacggtggcagccccacccaagggcgaggatgcagaggcccacaagaagagcaggatcaccagcgagggcgagtacatccccctggaccagatcgacatcaacgtgtaa

SEQ ID NO: 22 - protein, 421 aa, Artificial Sequence
  Description of Artificial Sequence Mela(palm)-mGluR6 L isoform of h melanopsin comprising a Golgi export signal protein
MNPPSGPRVLPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLFHPEQNVQKRKRSLKATSTVAAPPKGEDAEAHKKSRITSEGEYIPLDQIDINV

SEQ ID NO: 23 - DNA, 1266 bp, Artificial Sequence
  Description of Artificial Sequence Mela(palm)-mGlur6 P isoform of h melanopsin comprising a Golgi export signal cDNA
atgaaccctccttcggggccaagagtcccgcccagcccaacccaagagcccagctgcatggccaccccagcaccacccagctggtgggacagctcccagagcagcatctccagcctgggccggcttccatccatcagtcccacagcacctgggacttgggctgctgcctgggtccccctccccacggttgatgttccagaccatgcccactataccctgggcacagtgatcttgctggtgggactcacggggatgctgggcaacctgacggtcatctataccttctgcaggagcagaagcctccggacacctgccaacatgttcattatcaacctcgcggtcagcgacttcctcatgtccttcacccaggcccctgtcttcttcaccagtagcctctataagcagtggctctttggggagacaggctgcgagttctatgccttctgtggagctctctttggcatttcctccatgatcaccctgacggccatcgccctggaccgctacctggtaatcacacgcccgctggccacctttggtgtggcgtccaagaggcgtgcggcatttgtcctgctgggcgtttggctctatgccctggcctggagtctgccacccttcttcggctggagcgcctacgtgcccgaggggttgctgacatcctgctcctgggactacatgagcttcacgccggccgtgcgtgcctacaccatgcttctctgctgcttcgtgttcttcctccctctgcttatcatcatctactgctacatcttcatcttcagggccatccgggagacaggacgggctctccagaccttcggggcctgcaagggcaatggcgagtccctgtggcagcggcagcggctgcagagcgagtgcaagatggccaagatcatgctgctggtcatcctcctcttcgtgctctcctgggctccctattccgctgtggccctggtggcctttgctgggtacgcacacgtcctgacaccctacatgagctcggtgccagccgtcatcgccaaggcctctgcaatccacaaccccatcatttacgccatcacccaccccaagtacagggtggccattgcccagcacctgccctgcctgttccatccagagcagaatgtgcagaagcgaaagcggagcctcaaggccacctccacggtggcagccccacccaagggcgaggatgcagaggcccacaagaagagcaggatcaccagcgagggcgagtacatccccctggaccagatcgacatcaacgtgtaa

SEQ ID NO: 24 - protein, 421 aa, Artificial Sequence
  Description of Artificial Sequence Mela(palm)-mGlur6 P isoform of h melanopsin comprising a Golgi export signal protein
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLFHPEQNVQKRKRSLKATSTVAAPPKGEDAEAHKKSRITSEGEYIPLDQIDINV

SEQ ID NO: 25 - DNA, 1206 bp, Artificial Sequence
  Description of Artificial Sequence Mela(palm)-mGluR6 L isoform of h melanopsin without optional C-terminally added sequences cDNA
atgaaccctccttcggggccaagagtcctgcccagcccaacccaagagcccagctgcatggccaccccagcaccacccagctggtgggacagctcccagagcagcatctccagcctgggccggcttccatccatcagtcccacagcacctgggacttgggctgctgcctgggtccccctccccacggttgatgttccagaccatgcccactataccctgggcacagtgatcttgctggtgggactcacggggatgctgggcaacctgacggtcatctataccttctgcaggagcagaagcctccggacacctgccaacatgttcattatcaacctcgcggtcagcgacttcctcatgtccttcacccaggcccctgtcttcttcaccagtagcctctataagcagtggctctttggggagacaggctgcgagttctatgccttctgtggagctctctttggcatttcctccatgatcaccctgacggccatcgccctggaccgctacctggtaatcacacgcccgctggccacctttggtgtggcgtccaagaggcgtgcggcatttgtcctgctgggcgtttggctctatgccctggcctggagtctgccacccttcttcggctggagcgcctacgtgcccgaggggttgctgacatcctgctcctgggactacatgagcttcacgccggccgtgcgtgcctacaccatgcttctctgctgcttcgtgttcttcctccctctgcttatcatcatctactgctacatcttcatcttcagggccatccgggagacaggacgggctctccagaccttcggggcctgcaagggcaatggcgagtccctgtggcagcggcagcggctgcagagcgagtgcaagatggccaagatcatgctgctggtcatcctcctcttcgtgctctcctgggctccctattccgctgtggccctggtggcctttgctgggtacgcacacgtcctgacaccctacatgagctcggtgccagccgtcatcgccaaggcctctgcaatccacaaccccatcatttacgccatcacccaccccaagtacagggtggccattgcccagcacctgccctgcctgttccatccagagcagaatgtgcagaagcgaaagcggagcctcaaggccacctccacggtggcagccccacccaagggcgaggatgcagaggcccacaagtaa

SEQ ID NO: 26 - protein, 401 aa, Artificial Sequence
  Description of Artificial Sequence Mela(palm)-mGluR6 L isoform of h melanopsin without optional C-terminally added sequences protein
MNPPSGPRVLPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLFHPEQNVQKRKRSLKATSTVAAPPKGEDAEAHK

SEQ ID NO: 27 - DNA, 1206 bp, Artificial Sequence
  Description of Artificial Sequence Mela(palm)-mGluR6 P isoform of h melanopsin without optional C-terminally added sequences cDNA
atgaaccctccttcggggccaagagtcccgcccagcccaacccaagagcccagctgcatggccaccccagcaccacccagctggtgggacagctcccagagcagcatctccagcctgggccggcttccatccatcagtcccacagcacctgggacttgggctgctgcctgggtccccctccccacggttgatgttccagaccatgcccactataccctgggcacagtgatcttgctggtgggactcacggggatgctgggcaacctgacggtcatctataccttctgcaggagcagaagcctccggacacctgccaacatgttcattatcaacctcgcggtcagcgacttcctcatgtccttcacccaggcccctgtcttcttcaccagtagcctctataagcagtggctctttggggagacaggctgcgagttctatgccttctgtggagctctctttggcatttcctccatgatcaccctgacggccatcgccctggaccgctacctggtaatcacacgcccgctggccacctttggtgtggcgtccaagaggcgtgcggcatttgtcctgctgggcgtttggctctatgccctggcctggagtctgccacccttcttcggctggagcgcctacgtgcccgaggggttgctgacatcctgctcctgggactacatgagcttcacgccggccgtgcgtgcctacaccatgcttctctgctgcttcgtgttcttcctccctctgcttatcatcatctactgctacatcttcatcttcagggccatccgggagacaggacgggctctccagaccttcggggcctgcaagggcaatggcgagtccctgtggcagcggcagcggctgcagagcgagtgcaagatggccaagatcatgctgctggtcatcctcctcttcgtgctctcctgggctccctattccgctgtggccctggtggcctttgctgggtacgcacacgtcctgacaccctacatgagctcggtgccagccgtcatcgccaaggcctctgcaatccacaaccccatcatttacgccatcacccaccccaagtacagggtggccattgcccagcacctgccctgcctgttccatccagagcagaatgtgcagaagcgaaagcggagcctcaaggccacctccacggtggcagccccacccaagggcgaggatgcagaggcccacaagtaa

SEQ ID NO: 28 - protein, 401 aa, Artificial Sequence
  Description of Artificial Sequence Mela(palm)-mGluR6 P isoform of h melanopsin without optional C-terminally added sequences protein
MNPPSGPRVPPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLFHPEQNVQKRKRSLKATSTVAAPPKGEDAEAHK

SEQ ID NO: 29 - DNA, 1395 bp, Artificial Sequence
  Description of Artificial Sequence Mela(palm+33A)+mGluR6 (IL1, CT) cDNA
atgaaccctccttcggggccaagagtcctgcccagcccaacccaagagcccagctgcatggccaccccagcaccacccagctggtgggacagctcccagagcagcatctccagcctgggccggcttccatccatcagtcccacagcacctgggacttgggctgctgcctgggtccccctccccacggttgatgttccagaccatgcccactataccctgggcacagtgatcttgctggtgggactcacggggatgctgggcaacctgacggtcatctataccttccggtacaacaacacgcccatcgtccgggcctcgggccgagagctcttcattatcaacctcgcggtcagcgacttcctcatgtccttcacccaggcccctgtcttcttcaccagtagcctctataagcagtggctctttggggagacaggctgcgagttctatgccttctgtggagctctctttggcatttcctccatgatcaccctgacggccatcgccctggaccgctacctggtaatcacacgcccgctggccacctttggtgtggcgtccaagaggcgtgcggcatttgtcctgctgggcgtttggctctatgccctggcctggagtctgccacccttcttcggctggagcgcctacgtgcccgaggggttgctgacatcctgctcctgggactacatgagcttcacgccggccgtgcgtgcctacaccatgcttctctgctgcttcgtgttcttcctccctctgcttatcatcatctactgctacatcttcatcttcagggccatccgggagacaggacgggctctccagaccttcggggcctgcaagggcaatggcgagtccctgtggcagcggcagcggctgcagagcgagtgcaagatggccaagatcatgctgctggtcatcctcctcttcgtgctctcctgggctccctattccgctgtggccctggtggcctttgctgggtacgcacacgtcctgacaccctacatgagctcggtgccagccgtcatcgccaaggcctctgcaatccacaaccccatcatttacgccatcacccaccccaagtacagggtggccattgcccagcacctgccctgcctgggggtgctgctgggtgtatcacgccggcacagtcgcccctaccccagctaccgctccacccaccgctccacgctgaccagccacacctccaacctccatccagagcagaatgtgcagaagcgaaagcggagcctcaaggccacctccacggtggcagccccacccaagggcgaggatgcagaggcccacaagaagagcaggatcaccagcgagggcgagtacatccccctggaccagatcgacatcaacgtgacagagaccagccaagtggcgcctgcctaa

SEQ ID NO: 30 - protein, 464 aa, Artificial Sequence
  Description of Artificial Sequence Mela(palm+33A)+mGluR6 (IL1, CT) protein
  LIPID (370)...(370): PALMITATE
MNPPSGPRVLPSPTQEPSCMATPAPPSWWDSSQSSISSLGRLPSISPTAPGTWAAAWVPLPTVDVPDHAHYTLGTVILLVGLTGMLGNLTVIYTFRYNNTPIVRASGRELFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRALQTFGACKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLHPEQNVQKRKRSLKATSTVAAPPKGEDAEAHKKSRITSEGEYIPLDQIDINVTETSQVAPA

SEQ ID NO: 31 - DNA, 1389 bp, Artificial Sequence
  Description of Artificial Sequence Mela(palm+33A)+mGluR6 (IL3, CT) cDNA
atgaaccctccttcggggccaagagtcctgcccagcccaacccaagagcccagctgcatggccaccccagcaccacccagctggtgggacagctcccagagcagcatctccagcctgggccggcttccatccatcagtcccacagcacctgggacttgggctgctgcctgggtccccctccccacggttgatgttccagaccatgcccactataccctgggcacagtgatcttgctggtgggactcacggggatgctgggcaacctgacggtcatctataccttctgcaggagcagaagcctccggacacctgccaacatgttcattatcaacctcgcggtcagcgacttcctcatgtccttcacccaggcccctgtcttcttcaccagtagcctctataagcagtggctctttggggagacaggctgcgagttctatgccttctgtggagctctctttggcatttcctccatgatcaccctgacggccatcgccctggaccgctacctggtaatcacacgcccgctggccacctttggtgtggcgtccaagaggcgtgcggcatttgtcctgctgggcgtttggctctatgccctggcctggagtctgccacccttcttcggctggagcgcctacgtgcccgaggggttgctgacatcctgctcctgggactacatgagcttcacgccggccgtgcgtgcctacaccatgcttctctgctgcttcgtgttcttcctccctctgcttatcatcatctactgctacatcttcatcttcagggccatccgggagacaggacggggcgtgcccgagaccttcaacgaggccaagggcaatggcgagtccctgtggcagcggcagcggctgcagagcgagtgcaagatggccaagatcatgctgctggtcatcctcctcttcgtgctctcctgggctccctattccgctgtggccctggtggcctttgctgggtacgcacacgtcctgacaccctacatgagctcggtgccagccgtcatcgccaaggcctctgcaatccacaaccccatcatttacgccatcacccaccccaagtacagggtggccattgcccagcacctgccctgcctgggggtgctgctgggtgtatcacgccggcacagtcgcccctaccccagctaccgctccacccaccgctccacgctgaccagccacacctccaacctccatccagagcagaatgtgcagaagcgaaagcggagcctcaaggccacctccacggtggcagccccacccaagggcgaggatgcagaggcccacaagaagagcaggatcaccagcgagggcgagtacatccccctggaccagatcgacatcaacgtgacagagaccagccaagtggcgcctgcctaa

SEQ ID NO: 32 - protein, 462 aa, Artificial Sequence
  Description of Artificial Sequence Mela(palm+33A)+mGluR6 (IL3, CT) protein
  LIPID (368)...(368): PALMITATE
MNPPSGPRVLPSPTQEPSCMATPAPPSWWDSSQSSISSLGRLPSISPTAPGTWAAAWVPLPTVDVPDHAHYTLGTVILLVGLTGMLGNLTVIYTFCRSRSLRTPANMFIINLAVSDFLMSFTQAPVFFTSSLYKQWLFGETGCEFYAFCGALFGISSMITLTAIALDRYLVITRPLATFGVASKRRAAFVLLGVWLYALAWSLPPFFGWSAYVPEGLLTSCSWDYMSFTPAVRAYTMLLCCFVFFLPLLIIIYCYIFIFRAIRETGRGVPETFNEAKGNGESLWQRQRLQSECKMAKIMLLVILLFVLSWAPYSAVALVAFAGYAHVLTPYMSSVPAVIAKASAIHNPIIYAITHPKYRVAIAQHLPCLGVLLGVSRRHSRPYPSYRSTHRSTLTSHTSNLHPEQNVQKRKRSLKATSTVAAPPKGEDAEAHKKSRITSEGEYIPLDQIDINVTETSQVAPA

SEQ ID NO: 33 - DNA, 1095 bp, Artificial Sequence
  Description of Artificial Sequence JellyOP(palm)-mGluR6 cDNA
atgggagcgaatataacagaaatattgtcaggctttttagcttgtgtagtatttctctctataagcttgaatatgatagttctaattacattttaccgcttacgacataaattggcttttaaagatgctctcatggctagtatggcgttcagtgatgttgtacaagctatcgtaggatatcctttagaggtattcactgtagtagacggaaaatggacattcggaatggaattatgtcaagttgcaggatttttcattactgcccttggtcaagtttccatcgctcatctcactgctctcgccttagatcgatacttcacagtgtgcagacctttcgtggcaactgcgattcacggatcaatgagaaatgcaggtatggtcatatttgtttgctggttctacgcgtccttctgggcagtcctacccttagttggatggtcaaattatgacgtggagggtgacggtatgcgatgctccatcaactgggcagacgacagtcctaaaagctactcatacagggtttgtttattcgtattcatctacctgattcctgttcttttgatggtggctacttacgtgttggttcaaggagagatgaagaatatgcgaggtcgtgcagcacagttgtttggttcagaatccgaggctgcactaaagaatatcaaggccgaaaagcgacacacaaggctggttttcgtcatgatcctttctttcatcgtagcttggaccccatataccttcgtcgccatgtgggtttcctttttcacgaaacaacttgggccaatacctttatacgttgatactttggcagctatgcttgcaaagtcgtcggctatgttcaaccccatcatttactgcttcctccacaagcaattcagaagagctgtattacgtggtgtttgtctgttccatccagagcagaacgtgcagaagcggaagcgcagcctcaagaagacctccacgatggcggccccgcccaagagcgagaactcagaggacgccaagacagagaccagccaagtggcgcctgccaagagcaggatcaccagcgagggcgagtacatccccctggaccagatcgacatcaacgtgtaa

SEQ ID NO: 34 - protein, 364 aa, Artificial Sequence
  Description of Artificial Sequence JellyOP(palm)-mGluR6 protein
  LIPID (301)...(301): PALMITATE
MGANITEILSGFLACVVFLSISLNMIVLITFYRLRHKLAFKDALMASMAFSDVVQAIVGYPLEVFTVVDGKWTFGMELCQVAGFFITALGQVSIAHLTALALDRYFTVCRPFVATAIHGSMRNAGMVIFVCWFYASFWAVLPLVGWSNYDVEGDGMRCSINWADDSPKSYSYRVCLFVFIYLIPVLLMVATYVLVQGEMKNMRGRAAQLFGSESEAALKNIKAEKRHTRLVFVMILSFIVAWTPYTFVAMWVSFFTKQLGPIPLYVDTLAAMLAKSSAMFNPIIYCFLHKQFRRAVLRGVCLFHPEQNVQKRKRSLKKTSTMAAPPKSENSEDAKKSRITSEGEYIPLDQIDINVTETSQVAPA

SEQ ID NO: 35 - DNA, 1128 bp, Artificial Sequence
  Description of Artificial Sequence JellyOP(palm)-5HT7 cDNA
atgggagcgaatataacagaaatattgtcaggctttttagcttgtgtagtatttctctctataagcttgaatatgatagttctaattacattttaccgcttacgacataaattggcttttaaagatgctctcatggctagtatggcgttcagtgatgttgtacaagctatcgtaggatatcctttagaggtattcactgtagtagacggaaaatggacattcggaatggaattatgtcaagttgcaggatttttcattactgcccttggtcaagtttccatcgctcatctcactgctctcgccttagatcgatacttcacagtgtgcagacctttcgtggcaactgcgattcacggatcaatgagaaatgcaggtatggtcatatttgtttgctggttctacgcgtccttctgggcagtcctacccttagttggatggtcaaattatgacgtggagggtgacggtatgcgatgctccatcaactgggcagacgacagtcctaaaagctactcatacagggtttgtttattcgtattcatctacctgattcctgttcttttgatggtggctacttacgtgttggttcaaggagagatgaagaatatgcgaggtcgtgcagcacagttgtttggttcagaatccgaggctgcactaaagaatatcaaggccgaaaagcgacacacaaggctggttttcgtcatgatcctttctttcatcgtagcttggaccccatataccttcgtcgccatgtgggtttcctttttcacgaaacaacttgggccaatacctttatacgttgatactttggcagctatgcttgcaaagtcgtcggctatgttcaaccccatcatttactgcttcctccacaagcaattcagaagagctgtattacgtggtgtttgtcagtaccggaatatcaaccggaagctctctgctgcaggcatgcacgaagccctgaaacttgctgagaggcctgagagaagcgagtttgtgctacaaaactgtgaccactgtgggaaaaaaggtcatgatacatgaaagagcaggatcaccagcgagggcgagtacatccccctggaccagatcgacatcaacgtgacagagaccagccaagtggcgcctgcctaa

SEQ ID NO: 36 - protein, 374 aa, Artificial Sequence
  Description of Artificial Sequence JellyOP(palm)-5HT7 protein
  LIPID (301)...(301): PALMITATE
MGANITEILSGFLACVVFLSISLNMIVLITFYRLRHKLAFKDALMASMAFSDVVQAIVGYPLEVFTVVDGKWTFGMELCQVAGFFITALGQVSIAHLTALALDRYFTVCRPFVATAIHGSMRNAGMVIFVCWFYASFWAVLPLVGWSNYDVEGDGMRCSINWADDSPKSYSYRVCLFVFIYLIPVLLMVATYVLVQGEMKNMRGRAAQLFGSESEAALKNIKAEKRHTRLVFVMILSFIVAWTPYTFVAMWVSFFTKQLGPIPLYVDTLAAMLAKSSAMFNPIIYCFLHKQFRRAVLRGVCQYRNINRKLSAAGMHEALKLAERPERSEFVLQNCDHCGKKGHDTKSRITSEGEYIPLDQIDINVTETSQVAPA

SEQ ID NO: 37 - DNA, 1118 bp, Artificial Sequence
  Description of Artificial Sequence PPO(palm)-mGluR6 cDNA
ccatggagaacttgacctcgctcgacctcctgcccaacggcgaggtcccgttgatgccccgctacggcttcaccatcctcgccgtgatcatggccgtgttcaccatcgcctcgctcgtgctcaacagcaccgtcgtcatcgtcaccctgcgccaccgccagctacgccacccgctcaacttctcgctcgtcaacctcgccgtggcggacctgggcgtcacggtgttcggcgccagcctcgtcgtggagaccaacgccgtcgggtacttcaacctcggccgcgtcggctgcgtcatcgaagggttcgccgtcgctttcttcggcatcgccgctctgtgcacgatcgccgtgatcgccgtcgatcgcttcgtggtggtgtgcaagccgctgggcacgctgatgttcacgcggcgccacgcgctgctgggcatcgcctgggcctggctctggtcgttcgtgtggaacacgccgccgctcttcggctggggcagctacgagctggagggcgtgcggacgtcgtgcgcgcccgactggtacagccgcgaccccgccaacgtgtcgtacattacgagctacttcgccttctgcttcgccatccccttcctcgtcatcgtggtggcgtacggccgcctcatgtggaccctccaccaggtggccaagctggggatgggcgagagcggcagcaccgccaaggcggaggcgcaggtgtcgcgcatggtggtggtcatggtggtggccttcctcgtctgctggctgccctacgcgctcttcgccatgatcgtggtgaccaagcccgacgtgtacatcgacccggtcatcgccacactgcccatgtacctgaccaagacgagcacggtctacaaccccatcatctacatcttcatgaaccgccagttccgggactgcgccgtgcccttcctgctctgcctgttccatccagagcagaacgtgcagaagcggaagcgcagcctcaagaagacctccacgatggcggccccgcccaagagcgagaactcagaggacgccaagacagagaccagccaagtggcgcctgccaagagcaggatcaccagcgagggcgagtacatccccctggaccagatcgacatcaacgtgtaa

SEQ ID NO: 38 - protein, 371 aa, Artificial Sequence
  Description of Artificial Sequence PPO(palm)-mGluR6 protein
  LIPID (308)...(308): PALMITATE
MENLTSLDLLPNGEVPLMPRYGFTILAVIMAVFTIASLVLNSTVVIVTLRHRQLRHPLNFSLVNLAVADLGVTVFGASLVVETNAVGYFNLGRVGCVIEGFAVAFFGIAALCTIAVIAVDRFVVVCKPLGTLMFTRRHALLGIAWAWLWSFVWNTPPLFGWGSYELEGVRTSCAPDWYSRDPANVSYITSYFAFCFAIPFLVIVVAYGRLMWTLHQVAKLGMGESGSTAKAEAQVSRMVVVMVVAFLVCWLPYALFAMIVVTKPDVYIDPVIATLPMYLTKTSTVYNPIIYIFMNRQFRDCAVPFLLCLFHPEQNVQKRKRSLKKTSTMAAPPKSENSEDAKKSRITSEGEYIPLDQIDINVTETSQVAPA

SEQ ID NO: 39 - DNA, 1238 bp, Artificial Sequence
  Description of Artificial Sequence JSR1(palm)-mGluR6 cDNA
atgttaccacatgcagcaaaaatggcggccagggtggctggcgatcacgatggaagaaacatttcaattgttgatcttctgccagaagacatgctaccgatgattcacgaacattggtataagttccccccgatggaaacttccatgcattatatcctcggaatgcttattatagttataggaatcatcagtgtatcaggtaatggagttgtcatgtacttaatgatgacagtgaagaacctccgaactcccggtaattttctggtattgaatcttgccctatctgattttggtatgctgttttttatgatgccgacgatgtccataaattgcttcgccgaaacgtgggttataggacctttcatgtgtgagctctatggaatgatcggctcattatttggaagtgcatctatctggagtctggtaatgataacactcgaccgatacaatgtcatcgtgaaaggaatggctggaaaacctctaacgaaagttggagcactgttaagaatgcttttcgtctggatttggtcattaggctggaccattgcaccgatgtacggatggagcaggtatgttcctgaaggctcgatgacatcatgcactattgactacatagatacagctattaatccaatgtcttaccttatcgcctacgctatttttgtatacttcgtaccactctttatcattatttactgctacgctttcatcgtaatgcaagtagcagcccacgagaaatccctacgagaacaagctaaaaagatgaacatcaaatccctcagatcaaatgaagataacaagaaagcaagtgcagaattcagactggctaaggtggcttttatgaccatctgctgctggttcatggcgtggactccttatctaaccctgtccttccttggaatcttctccgacaggacctggctaacacctatgacatccgtttggggagccatatttgcaaaagctagtgcctgctacaatcctattgtttatggaataagtcatcctaagtatcgtgccgctttacatgataagttcccatgcctctgttccatccagagcagaacgtgcagaagcggaagcgcagcctcaagaagacctccacgatggcggccccgcccaagagcgagaactcagaggacgccaagacagagaccagccaagtggcgcctgccaagagcaggatcaccagcgagggcgagtacatccccctggaccagatcgacatcaacgtgtaa

SEQ ID NO: 40 - protein, 411 aa, Artificial Sequence
  Description of Artificial Sequence JSR1(palm)-mGluR6 protein
  LIPID (348)...(348): PALMITATE
MLPHAAKMAARVAGDHDGRNISIVDLLPEDMLPMIHEHWYKFPPMETSMHYILGMLIIVIGIISVSGNGVVMYLMMTVKNLRTPGNFLVLNLALSDFGMLFFMMPTMSINCFAETWVIGPFMCELYGMIGSLFGSASIWSLVMITLDRYNVIVKGMAGKPLTKVGALLRMLFVWIWSLGWTIAPMYGWSRYVPEGSMTSCTIDYIDTAINPMSYLIAYAIFVYFVPLFIIIYCYAFIVMQVAAHEKSLREQAKKMNIKSLRSNEDNKKASAEFRLAKVAFMTICCWFMAWTPYLTLSFLGIFSDRTWLTPMTSVWGAIFAKASACYNPIVYGISHPKYRAALHDKFPCLFHPEQNVQKRKRSLKKTSTMAAPPKSENSEDAKKSRITSEGEYIPLDQIDINVTETSQVAPA

SEQ ID NO: 41 - DNA, 1722 bp, Artificial Sequence
  Description of Artificial Sequence hJSR(S186F)palm-GABAB2-TS-1D4 cDNA
atgctgcctcacgccgctaaaatggccgcaagagtggccggagatcacgacggcagaaacatctctatcgtggacctgctgcctgaagatatgcttccaatgattcacgagcactggtacaagtttccccccatggaaaccagcatgcactacatcctgggcatgctgatcatcgtgatcggaatcatttctgtgtccggcaatggcgtggtgatgtacctgatgatgaccgttaagaacctgcggacacctggaaatttcctggtgctgaacctggctctgagcgacttcggcatgctgttcttcatgatgcccaccatgagcatcaactgtttcgccgagacatgggtcatcggtcctttcatgtgcgagctgtacggcatgatcggctctctgtttggcagcgccagcatctggtctcttgtgatgatcaccctggaccggtataatgtcatcgttaagggaatggccggaaagcctctgaccaaggtgggcgctctgctgagaatgctcttcgtgtggatctggtccctgggctggaccattgcccctatgtacggctggagcagatatgtgccagagggctctatgaccttctgcaccatcgattacatcgacaccgccattaaccccatgagctacctgatcgcctacgccatcttcgtgtacttcgtgcccctgttcatcatcatctactgctacgcctttatcgtgatgcaggtggctgcccatgagaagagcctgcgggaacaggccaagaaaatgaacatcaagagcctgcgctccaacgaggacaacaagaaggcctctgccgagttcagactggccaaagtggccttcatgaccatctgctgttggttcatggcttggaccccttacctgacactgagcttcctgggcatcttcagcgacagaacatggctgacccctatgacaagcgtgtggggcgccatctttgccaaggccagcgcctgttacaaccctatcgtgtacggcatcagccaccccaagtacagagccgccctgcacgataagtttccttgcactctgaggacaaaccctgacgcagccactcagaacaggcgattccagttcacacagaaccagaagaaagaagattcgaagacctccacctcagtcaccagcgtgaaccaggcgagcacgtcacgcctggagggactgcagtcagaaaaccaccgcctacgaatgaagatcacagagctggacaaagacttggaagaagtcaccatgcagctacaagacacaccagagaagaccacatacatcaaacagaatcactaccaagagctcaacgacatcctcagcttgggcaacttcacagagagcacagatggaggaaaggccattctaaaaaatcacctcgatcaaaaccctcagctccagtggaacacgaccgagccctcaagaacatgcaaagaccccatagaggacatcaactccccagagcacatccagcgccggctgtccctccagctccccatcctccaccacgcctacctcccgtccattggaggcgtggacgccagctgcgtcagcccctgtgtcagccctacagccagccctcgccacagacacgtaccaccctccttccgagtcatggtctcgggcctgaagagcaggatcaccagcgagggcgagtacatccccctggaccagatcgacatcaacgtgacagagaccagccaagtggcgcctgcctaa

SEQ ID NO: 42 - protein, 573 aa, Artificial Sequence
  Description of Artificial Sequence hJSR(S186F)palm-GABAB2-TS-1D4 protein
MLPHAAKMAARVAGDHDGRNISIVDLLPEDMLPMIHEHWYKFPPMETSMHYILGMLIIVIGIISVSGNGVVMYLMMTVKNLRTPGNFLVLNLALSDFGMLFFMMPTMSINCFAETWVIGPFMCELYGMIGSLFGSASIWSLVMITLDRYNVIVKGMAGKPLTKVGALLRMLFVWIWSLGWTIAPMYGWSRYVPEGSMTFCTIDYIDTAINPMSYLIAYAIFVYFVPLFIIIYCYAFIVMQVAAHEKSLREQAKKMNIKSLRSNEDNKKASAEFRLAKVAFMTICCWFMAWTPYLTLSFLGIFSDRTWLTPMTSVWGAIFAKASACYNPIVYGISHPKYRAALHDKFPCTLRTNPDAATQNRRFQFTQNQKKEDSKTSTSVTSVNQASTSRLEGLQSENHRLRMKITELDKDLEEVTMQLQDTPEKTTYIKQNHYQELNDILSLGNFTESTDGGKAILKNHLDQNPQLQWNTTEPSRTCKDPIEDINSPEHIQRRLSLQLPILHHAYLPSIGGVDASCVSPCVSPTASPRHRHVPPSFRVMVSGLKSRITSEGEYIPLDQIDINVTETSQVAPA

SEQ ID NO: 43 - DNA, 1374 bp, Artificial Sequence
  Description of Artificial Sequence JSR(S186F)palm-beta2AR cDNA
atgctgcctcacgccgctaaaatggccgcaagagtggccggagatcacgacggcagaaacatctctatcgtggacctgctgcctgaagatatgcttccaatgattcacgagcactggtacaagtttccccccatggaaaccagcatgcactacatcctgggcatgctgatcatcgtgatcggaatcatttctgtgtccggcaatggcgtggtgatgtacctgatgatgaccgttaagaacctgcggacacctggaaatttcctggtgctgaacctggctctgagcgacttcggcatgctgttcttcatgatgcccaccatgagcatcaactgtttcgccgagacatgggtcatcggtcctttcatgtgcgagctgtacggcatgatcggctctctgtttggcagcgccagcatctggtctcttgtgatgatcaccctggaccggtataatgtcatcgttaagggaatggccggaaagcctctgaccaaggtgggcgctctgctgagaatgctcttcgtgtggatctggtccctgggctggaccattgcccctatgtacggctggagcagatatgtgccagagggctctatgaccttctgcaccatcgattacatcgacaccgccattaaccccatgagctacctgatcgcctacgccatcttcgtgtacttcgtgcccctgttcatcatcatctactgctacgcctttatcgtgatgcaggtggctgcccatgagaagagcctgcgggaacaggccaagaaaatgaacatcaagagcctgcgctccaacgaggacaacaagaaggcctctgccgagttcagactggccaaagtggccttcatgaccatctgctgttggttcatggcttggaccccttacctgacactgagcttcctgggcatcttcagcgacagaacatggctgacccctatgacaagcgtgtggggcgccatctttgccaaggccagcgcctgttacaaccctatcgtgtacggcatcagccaccccaagtacagagccgccctgcacgataagtttccttgcattgccttccaggagcttctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctgaagagcaggatcaccagcgagggcgagtacatccccctggaccagatcgacatcaacgtgacagagaccagccaagtggcgcctgcctaa

SEQ ID NO: 44 - protein, 457 aa, Artificial Sequence
  Description of Artificial Sequence JSR(S186F)palm-beta2AR protein
MLPHAAKMAARVAGDHDGRNISIVDLLPEDMLPMIHEHWYKFPPMETSMHYILGMLIIVIGIISVSGNGVVMYLMMTVKNLRTPGNFLVLNLALSDFGMLFFMMPTMSINCFAETWVIGPFMCELYGMIGSLFGSASIWSLVMITLDRYNVIVKGMAGKPLTKVGALLRMLFVWIWSLGWTIAPMYGWSRYVPEGSMTFCTIDYIDTAINPMSYLIAYAIFVYFVPLFIIIYCYAFIVMQVAAHEKSLREQAKKMNIKSLRSNEDNKKASAEFRLAKVAFMTICCWFMAWTPYLTLSFLGIFSDRTWLTPMTSVWGAIFAKASACYNPIVYGISHPKYRAALHDKFPCIAFQELLCLRRSSLKAYGNGYSSNGNTGEQSGYHVEQEKENKLLCEDLPGTEDFVGHQGTVPSDNIDSQGRNCSTNDSLLKSRITSEGEYIPLDQIDINVTETSQVAPA

SEQ ID NO: 45 - protein, 56 aa, Artificial Sequence
  Description of Artificial Sequence Amino Acid Sequence at Splicing Site palm Mela hOPN4 trunc CT mGlur6 CT
NPIIYAITHPKYRVAIAQHLPCLFHPEQNVQKRKRSLKKTSTMAAPPKSENSEDAK

SEQ ID NO: 46 - protein, 79 aa, Artificial Sequence
  Description of Artificial Sequence Amino Acid Sequence at Splicing Site palm+33 Mela hOPN4 trunc CT mGlur6 CT
HPKYRVAIAQHLPCLGVLLGVSGQRSHPSLSYRSTHRSTLSSQSSDLHPEQNVQKRKRSLKKTSTMAAPPKSENSEDAK

SEQ ID NO: 47 - protein, 40 aa, Artificial Sequence
  Description of Artificial Sequence Amino Acid Sequence at Splicing Site Jct a Jct b Mela hOPN4 TM1 TM2 mGlur6 IL1
GMLGNLTVIYTFMRHNDTPIVRASGRELFIINLAVSDFLM

SEQ ID NO: 48 - protein, 47 aa, Artificial Sequence
  Description of Artificial Sequence Amino Acid Sequence at Splicing Site variable region Mela hOPN4 proxim IL3 region distal IL3 region mGlur6 IL3
IFIFRAIRETGRGVPETFNEAPLRQRRQWQRLQSEWKMAKVALIVIL

SEQ ID NO: 49 - protein, 66 aa, Artificial Sequence
  Description of Artificial Sequence Amino Acid Sequence at Splicing Site palm Jellyopsin Cubop trunc CT m5HT7 CT
FNPIIYCFLHKQFRRAVLRGVCQYRNINRKLSAAGMHEALKLAERPERSEFVLQNCDHCGKKGHDT

SEQ ID NO: 50 - protein, 56 aa, Artificial Sequence
  Description of Artificial Sequence Amino Acid Sequence at Splicing Site palm JellyOP cubop trunc CT mGlur6 CT
FNPIIYCFLHKQFRRAVLRGVCLFHPEQNVQKRKRSLKKTSTMAAPPKSENSEDAK

SEQ ID NO: 51 - protein, 53 aa, Artificial Sequence
  Description of Artificial Sequence Amino Acid Sequence at Splicing Site palm Cone opsin hOPN1MW trunc CT mGlur6 CT
YNPIIYVFMNRQFRNCILHLFHPEQNVQKRKRSLKKTSTMAAPPKSENSEDAK

SEQ ID NO: 52 - protein, 49 aa, Artificial Sequence
  Description of Artificial Sequence Amino Acid Sequence at Splicing Site palm Mela hOPN4 trunc CT Cone opsin OPN1MW CT
NPIIYAITHPKYRVAIAQHLPCGKKVDDSSELSSTSKTEVSSVSSVSPA

SEQ ID NO: 53 - protein, 8 aa, Artificial Sequence
  Description of Artificial Sequence 1D4 tag
ETSQVAPA

SEQ ID NO: 54 - protein, 36 aa, Artificial Sequence
  Description of Artificial Sequence Amino Acid Sequence at Splicing Site Jct a Jct b Mela hOPN4 TM1 TM2 Cone opsin OPN1MW IL1
TGMLGNLTVIYATMKFKKLRHPANMFIINLAVSDFL

SEQ ID NO: 55 - protein, 44 aa, Artificial Sequence
  Description of Artificial Sequence Amino Acid Sequence at Splicing Site Jct c Jct d Mela hOPN4 TM3 TM4 Cone opsin OPN1MW IL2
TAIALDRYLVICKPFGNVRFDAKLTALVLLGVWLYALAWSLPPF

SEQ ID NO: 56 - protein, 47 aa, Artificial Sequence
  Description of Artificial Sequence Amino Acid Sequence at Splicing Site Jct e Jct f Mela hOPN4 TM5 TM6 Cone opsin OPN1MW IL3
LIIIFCYIQVWLAIRAVAKQQKESESTQKAEKEVTKVALIVILLFVL

SEQ ID NO: 57 - protein, 125 aa, Homo sapiens
  Sequence section human melanopsin gene (OPN4) C-terminal melanopsin fragment
HPKYRVAIAQHLPCLGVLLGVSRRHSRPYPSYRSTHRSTLTSHTSNLSWISIRRRQESLGSESEVGWTHMEAAAVWGAAQQANGRSLYGQGLEDLEAKAPPRPQGHEAETPGKTKGLIPSQDPRM

SEQ ID NO: 58 - protein, 116 aa, Mus sp.
  Sequence section murine melanopsin gene (OPN4) C-terminal melanopsin fragment
HPKYRVAIAQHLPCLGVLLGVSGQRSHPSLSYRSTHRSTLSSQSSDLSWISGRKRQESLGSESEVGWTDTETTAAWGAAQQASGQSFCSQNLEDGELKASSSPQTKGHLPSLDLGM

SEQ ID NO: 59 - protein, 735 aa, Adeno-associated virus 2
  wild type capsid AAV2 sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 60 - protein, 7 aa, Artificial Sequence
  Description of Artificial Sequence peptide insert SASEAST
SASEAST

SEQ ID NO: 61 - protein, 7 aa, Artificial Sequence
  Description of Artificial Sequence peptide insert TPPSITA
TPPSITA

SEQ ID NO: 62 - protein, 7 aa, Artificial Sequence
  Description of Artificial Sequence peptide insert PRTPHTA
PRTPHTA

SEQ ID NO: 63 - protein, 7 aa, Artificial Sequence
  Description of Artificial Sequence peptide insert NHAPNHC
NHAPNHC

SEQ ID NO: 64 - protein, 12 aa, Artificial Sequence
  Description of Artificial Sequence peptide insert AAASASEASTAA
AAASASEASTAA

SEQ ID NO: 65 - protein, 12 aa, Artificial Sequence
  Description of Artificial Sequence peptide insert AAATPPSITAAA
AAATPPSITAAA

SEQ ID NO: 66 - protein, 12 aa, Artificial Sequence
  Description of Artificial Sequence peptide insert AAAPRTPHTAAA
AAAPRTPHTAAA

SEQ ID NO: 67 - protein, 12 aa, Artificial Sequence
  Description of Artificial Sequence peptide insert NLANHAPNHCAR
NLANHAPNHCAR

SEQ ID NO: 68 - protein, 12 aa, Artificial Sequence
  Description of Artificial Sequence peptide insert NLAPRTPHTAAR
NLAPRTPHTAAR

SEQ ID NO: 69 - protein, 37 aa, Artificial Sequence
  Description of Artificial Sequence peptide insert SASEAST with linker
VSTNLQRGNAAASASEASTAARQAATADVNTQGVLPG

SEQ ID NO: 70 - protein, 37 aa, Artificial Sequence
  Description of Artificial Sequence peptide insert TPPSITA with linker
VSTNLQRGNAAATPPSITAAARQAATADVNTQGVLPG

SEQ ID NO: 71 - protein, 37 aa, Artificial Sequence
  Description of Artificial Sequence peptide insert PRTPHTA with linker
VSTNLQRGNAAAPRTPHTAAARQAATADVNTQGVLPG

SEQ ID NO: 72 - protein, 37 aa, Artificial Sequence
  Description of Artificial Sequence peptide insert NHAPNHC with linker
VSTNLQRGNNLANHAPNHCARRQAATADVNTQGVLPG

SEQ ID NO: 73 - protein, 37 aa, Artificial Sequence
  Description of Artificial Sequence peptide insert PRTPHTA-AR with linker
VSTNLQRGNNLAPRTPHTAARRQAATADVNTQGVLPG

SEQ ID NO: 74 - protein, 747 aa, Artificial Sequence
  Description of Artificial Sequence AAV capsid with NLAPRTPHTAAR insertion
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTFNNHLYKQISSQSGASNDNHFFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNNLAPRTPHTAARRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQFTSNYNKSVNVDFTVDTNGVYSEPRPIGTRFLTRNL

SEQ ID NO: 75 - DNA, 1354 bp, Artificial Sequence
  Description of Artificial Sequence ON-Bipolar cell specific promoter 770En_566P(hGRM6)
gggtctccaacttgccaactgtagatcttggaacctttcatccttcataactgcataagccaattccttctaataaatctgtataatatatctgtctatataataaatatgtacttacataactctgtatgttacatctatctattctatctatctatctatctatctatctatctatctatctatcatctatctatctatttatctattctctgtgtctttggagaaccctgacatagtaagcaatcatatcacctgcaaatgatgaaagctgtgtattttccaaatcagtcgttttatgtctttttttcttgcactgactagtgccccctagagggaatgataattggaattattgtcttgctctgattttaaaggaagtagatacttcaaataattcatcatggagtgcaatattttctgtaggcttttagtagataacttcatcagtttaaagaagatccttagattatgaaacatttacaattatgaatgaatattagatgttatcaaatgctttttctgcatccatttagataatcatgtttttcctttaatctgttaatgcggtgaattacattaatagatttcctaagtcattaatctgctaaagtgcatttctgggacaaaccagacttggttatgacattgtatgtatttcagtttgcaaatattggactaggatttttgtatctatattccttagtttgacctgtaaattttatttcttgtactaagtattagcctcacgaaaggcattgtcaaatgttcaattgatataatgctagccaagaagaggacagaggcagaaagccagggacagagactgagaaacagagacctagaggcagaagaagactgagatagagatggacagagattgtgtcagacacagccccagagacagccagacagtctgagtcagacgcaaaccaaagacaagaaaacaggaaaacagacccagagattgggagagggaggggaaggagatgcggggagagccagcaccgccaccccccacactcaggaggggtctccaccctcggagcggtctctcatccctccctagaatccttaaatcctctctcgctcagggcctcggccgcatctgtcacagacttgtcctgaaccgacagcggctggcgcaggtgactggcttggggcgggagcctgggtgtgcgctggggatggaccccgaggaagaggggccaagctgtcgggaagcggcagggctggaggggtggaggcagtggtcgggcgggaccccgggcgacagggttcggcgcttgtaagagcgagacggaggcccgggcaggccggctgagctaactccccagagccgaagtggaaggcg

SEQ ID NO: 76 - DNA, 917 bp, Artificial Sequence
  Description of Artificial Sequence ON-Bipolar cell specific promoter 444En_454P(hGRM6)
atctattctctgtgtctttggagaaccctgacatagtaagcaatcatatcacctgcaaatgatgaaagctgtgtattttccaaatcagtcgttttatgtctttttttcttgcactgactagtgccccctagagggaatgataattggaattattgtcttgctctgattttaaaggaagtagatacttcaaataattcatcatggagtgcaatattttctgtaggcttttagtagataacttcatcagtttaaagaagatccttagattatgaaacatttacaattatgaatgaatattagatgttatcaaatgctttttctgcatccatttagataatcatgtttttcctttaatctgttaatgcggtgaattacattaatagatttcctaagtcattaatctgctaaagtgcatttctgggacaaaccagacttggttatgaccaattgatataatgctagcggaggggtctccaccctcggagcggtctctcatccctccctagaatccttaaatcctctctcgctcagggcctcggccgcatctgtcacagacttgtcctgaaccgacagcggctggcgcaggtgactggcttggggcgggagcctgggtgtgcgctggggatggaccccgaggaagaggggccaagctgtcgggaagcggcagggctggaggggtggaggcagtggtcgggcgggaccccgggcgacagggttcggcgcttgtaagagcgagacggaggcccgggcaggccggctgagctaactccccagagccgaagtggaaggcgcgccccgagcgccttctccccaggaccccggtgtccctccccgcgccccgagcccgcgctctccttcccccgccctcagagcgctccccgcccctctgtctccccgcagcccgctagacgagccga

SEQ ID NO: 77 - RNA, 13 nt, Artificial Sequence
  Description of Artificial Sequence KOZAK consensus sequence 1
gccgccaccaugg

SEQ ID NO: 78 - RNA, 13 nt, Artificial Sequence
  Description of Artificial Sequence KOZAK consensus sequence 2
gccgccgccaugg

SEQ ID NO: 79 - DNA, 6020 bp, Artificial Sequence
  Description of Artificial Sequence rAAV vector OPN4-mGluR6-TS-1D4
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagtctagagggtctccaacttgccaactgtagatcttggaacctttcatccttcataactgcataagccaattccttctaataaatctgtataatatatctgtctatataataaatatgtacttacataactctgtatgttacatctatctattctatctatctatctatctatctatctatctatctatctatcatctatctatctatttatctattctctgtgtctttggagaaccctgacatagtaagcaatcatatcacctgcaaatgatgaaagctgtgtattttccaaatcagtcgttttatgtctttttttcttgcactgactagtgccccctagagggaatgataattggaattattgtcttgctctgattttaaaggaagtagatacttcaaataattcatcatggagtgcaatattttctgtaggcttttagtagataacttcatcagtttaaagaagatccttagattatgaaacatttacaattatgaatgaatattagatgttatcaaatgctttttctgcatccatttagataatcatgtttttcctttaatctgttaatgcggtgaattacattaatagatttcctaagtcattaatctgctaaagtgcatttctgggacaaaccagacttggttatgacattgtatgtatttcagtttgcaaatattggactaggatttttgtatctatattccttagtttgacctgtaaattttatttcttgtactaagtattagcctcacgaaaggcattgtcaaatgttcaattgatataatgctagcggaggggtctccaccctcggagcggtctctcatccctccctagaatccttaaatcctctctcgctcagggcctcggccgcatctgtcacagacttgtcctgaaccgacagcggctggcgcaggtgactggcttggggcgggagcctgggtgtgcgctggggatggaccccgaggaagaggggccaagctgtcgggaagcggcagggctggaggggtggaggcagtggtcgggcgggaccccgggcgacagggttcggcgcttgtaagagcgagacggaggcccgggcaggccggctgagctaactccccagagccgaagtggaaggcgcgccccgagcgccttctccccaggaccccggtgtccctccccgcgccccgagcccgcgctctccttcccccgccctcagagcgctccccgcccctctgtctccccgcagcccgctagacgagccgagcggccgcgccaccatgaaccctccttcggggccaagagtcccgcccagcccaacccaagagcccagctgcatggccaccccagcaccacccagctggtgggacagctcccagagcagcatctccagcctgggccggcttccatccatcagtcccacagcacctgggacttgggctgctgcctgggtccccctccccacggttgatgttccagaccatgcccactataccctgggcacagtgatcttgctggtgggactcacggggatgctgggcaacctgacggtcatctataccttctgcaggagcagaagcctccggacacctgccaacatgttcattatcaacctcgcggtcagcgacttcctcatgtccttcacccaggcccctgtcttcttcaccagtagcctctataagcagtggctctttggggagacaggctgcgagttctatgccttctgtggagctctctttggcatttcctccatgatcaccctgacggccatcgccctggaccgctacctggtaatcacacgcccgctggccacctttggtgtggcgtccaagaggcgtgcggcatttgtcctgctgggcgtttggctctatgccctggcctggagtctgccacccttcttcggctggagcgcctacgtgcccgaggggttgctgacatcctgctcctgggactacatgagcttcacgccggccgtgcgtgcctacaccatgcttctctgctgcttcgtgttcttcctccctctgcttatcatcatctactgctacatcttcatcttcagggccatccgggagacaggacgggctctccagaccttcggggcctgcaagggcaatggcgagtccctgtggcagcggcagcggctgcagagcgagtgcaagatggccaagatcatgctgctggtcatcctcctcttcgtgctctcctgggctccctattccgctgtggccctggtggcctttgctgggtacgcacacgtcctgacaccctacatgagctcggtgccagccgtcatcgccaaggcctctgcaatccacaaccccatcatttacgccatcacccaccccaagtacagggtggccattgcccagcacctgccctgcctgttccatccagagcagaatgtgcagaagcgaaagcggagcctcaaggccacctccacggtggcagccccacccaagggcgaggatgcagaggcccacaagaagagcaggatcaccagcgagggcgagtacatccccctggaccagatcgacatcaacgtgacagagaccagccaagtggcgcctgcctaaggatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 80 - protein, 3 aa, Artificial Sequence
  Description of Artificial Sequence E(D)RY motif
  misc_feature (1)...(1): E or D
XRY

SEQ ID NO: 81 - protein, 5 aa, Artificial Sequence
  Description of Artificial Sequence NPxxY motif
  misc_feature (3)...(4): Any amino acid
NPXXY

SEQ ID NO: 82 - protein, 3 aa, Artificial Sequence
  Description of Artificial Sequence NR(K)Q motif
  misc_feature (2)...(2): R or K
NXQ

SEQ ID NO: 83 - protein, 4 aa, Artificial Sequence
  Description of Artificial Sequence DRIY variant of E(D)RY motif
DRIY

SEQ ID NO: 84 - protein, 4 aa, Artificial Sequence
  Description of Artificial Sequence NRIY variant of E(D)RY motif
NRIY

SEQ ID NO: 85 - protein, 20 aa, Artificial Sequence
  Description of Artificial Sequence Golgi export signal from the potassium channel Kir2.1
KSRITSEGEYIPLDQIDINV

SEQ ID NO: 86 - protein, 7 aa, Artificial Sequence
  Description of Artificial Sequence ER export signal from Kir2.1
FCYENEV

SEQ ID NO: 87 - protein, 6 aa, Artificial Sequence
  Description of Artificial Sequence Synthetic peptide
NRQFRN

SEQ ID NO: 88 - protein, 6 aa, Artificial Sequence
  Description of Artificial Sequence Synthetic peptide
CILHLF

SEQ ID NO: 89 - protein, 6 aa, Artificial Sequence
  Description of Artificial Sequence Synthetic peptide
QYRNIN

SEQ ID NO: 90 - protein, 4 aa, Artificial Sequence
  Description of Artificial Sequence Synthetic peptide
IAFQ

SEQ ID NO: 91 - protein, 12 aa, Artificial Sequence
  Description of Artificial Sequence Synthetic peptide
  misc_feature (3)...(4): Any amino acid
  misc_feature (6)...(11): Any amino acid
  SITE (6)...(11): This region may encompass 5-6 residues
NPXXYXXXXXXF

SEQ ID NO: 92 - protein, 87 aa, Unknown
  Description of Unknown X. Opn4x sequence
LCCCVFFIPLIIISHCYFFMFLAIRSTGRQRSVQKEQQVEARGFTCSSGGIQKRELGKSPLYVIVVLTLKGSITNHYTLKSYMGHGK

SEQ ID NO: 93 - protein, 85 aa, Unknown
  Description of Unknown Opn4xa sequence
LCCFVFFIPLGIISYCYFFMFLAIRSASRDVEKLGSHVRKTTLIQQQSIKEWKLAKIAFVVIIVFVLSWSPYACVTLIAWAGYSH

SEQ ID NO: 94 - protein, 83 aa, Unknown
  Description of Unknown Opn4xb sequence
LCCFVFFIPLSIILYCYLFMFLSVRQASRDLEMLSSQKSSFVKQQSMREWKLAKIAAVVIVVYVLSWAPYACVTLVAWAGHQD

SEQ ID NO: 95 - protein, 84 aa, Unknown
  Description of Unknown M. Opn4 sequence
LFCFVFFLPLLIIIFCYIFIFRAIRETGRACEGCGESPLRQRRQWQRLQEWKMAKVALIVILLFVLSWAPYSTVALVAFAGYSH

SEQ ID NO: 96 - protein, 85 aa, Unknown
  Description of Unknown Opn4a sequence
LFIFVFFIPLIVIIYCYFFIFRSIRTTNEAVGKINGDNKRDSMKRFQRLKEWKMAKIALIVILMYVISWSPYSTVALTAFAGYSD

SEQ ID NO: 97 - protein, 86 aa, Unknown
  Description of Unknown Opn4b sequence
LFTFVFFIPLFVIIYCYFFIFKAIRETNRAVGKINGEGGPRDSIKKIHRMKEWKMAKIALIVILLYVISWSPYSCVALTAFAGYAD

SEQ ID NO: 98 - protein, 82 aa, Unknown
  Description of Unknown Opn4.1 sequence
LFVFVFFIPLGIIGSCYFAIFQTIRAAGKEIRELDCGETHKVYERMQEWKMAKVALVVIVLFIISWSPYSVVALTATAGYSH

SEQ ID NO: 99 - protein, 45 aa, Artificial Sequence
  Description of Artificial Sequence Synthetic polypeptide
IFIFRAIRETGRGVPETFNEAPLRQRRQWQRLQSEWKMAKVALIV